SEQUENCE LISTING (8 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 805 aa, Homo sapiens [sequence omitted]

SEQ ID NO: 3 - protein, 1044 aa, synthetic construct
MLLVNQSHQGFNKEHTSKMVSAIVLYVLLAAAAHSAFAGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFSGGGGSGGGGSSGGENLYFQGGSGGGGSGGGGSSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGLNDIFEAQKIEWHEGGHHHHHH

SEQ ID NO: 4 - protein, 888 aa, synthetic construct
MGWSCIILFLVATATGVHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSGLNDIFEAQKIEWHEGGSENLYFQGGSGGGGSGGGGSSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAGGHHHHHH

SEQ ID NO: 5 - protein, 888 aa, synthetic construct
MGWSCIILFLVATATGVHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYCFQPTNGVGYQPYRVVVLSFELLHAPATVCGPKKSTNLVKNKCVNFGGSGLNDIFEAQKIEWHEGGSENLYFQGGSGGGGSGGGGSSQSTIEEQAKTFLDKFNHEAECLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAGGHHHHHH

SEQ ID NO: 6 - protein, 5 aa, synthetic construct
GGGGS

SEQ ID NO: 7 - protein, 10 aa, synthetic construct
GGGGSGGGGS

SEQ ID NO: 8 - protein, 6 aa, synthetic construct
HHHHHH

(19) United States
(12) Patent Application Publication
(10) Pub. No.: US 2024/0361333 A1
(43) Pub. Date: Oct. 31, 2024

(54) PLATFORMS FOR ANTIBODY SUBPOPULATION DETECTION

(71) Applicant: Autonomous Medical Devices Incorporated, Santa Ana, CA (US)

(72) Inventors: Ralph Eugene DAVIS, Mountain View, CA (US); Humaira JANWARI, Palo Alto, CA (US); Ji Youn LIM, Palo Alto, CA (US); Aravind SRINIVASAN, Lexington, MA (US); David OKRONGLY, Costa Mesa, CA (US); Sasha PEREBIKOVSKY, Irvine, CA (US); Brandon POLLACK, Lake Forest, CA (US); Marjorie CAPARAS, Costa Mesa, CA (US); Alex HWU, Anaheim, CA (US)

(21) Appl. No.: 18/769,201

(22) Filed: Jul. 10, 2024

Related U.S. Application Data
(63) Continuation of application No. PCT/US2023/010495, filed on Jan. 10, 2023.
(60) Provisional application No. 63/298,561, filed on Jan. 11, 2022.
(60) Provisional application No. 63/393,722, filed on Jul. 29, 2022.

(51) Int. Cl. G01N 33/68; C07K 14/005; C12N 7/00; G01N 33/569

(52) U.S. Cl. CPC G01N 33/6854; C07K 14/005; C12N 7/00; G01N 33/56983; C12N 2770/20022; G01N 2333/165

(57) ABSTRACT

Disclosed herein are methods, compositions, and devices for the detection of neutralizing antibodies for a pathogen of interest. In some embodiments, a covalent coupling between a viral protein and a receptor protein is utilized to reduce dissociation of the viral protein/receptor complex.

### CROSS-REFERENCE TO RELATED APPLICATIONS

This application is a continuation of International Application No. PCT/US2023/010495, filed Jan. 10, 2023, which claims the benefit of U.S. Provisional Patent Application No. 63/298,561, filed on Jan. 11, 2022, and U.S. Provisional Patent Application No. 63/393,722, filed on Jul. 29, 2022, each of which is incorporated herein by reference in its entirety.

### REFERENCE TO A SEQUENCE LISTING

The instant application contains a Sequence Listing which has been submitted electronically in XML format and is hereby incorporated by reference in its entirety. Said XML file, created on Apr. 5, 2023, is named AMDI 61070-701.301 SL.xml and is 12,854 bytes in size.

### BACKGROUND

Following exposure to a pathogen, immune systems often produce antibodies which recognize and bind molecular manifolds specific to the pathogen. Antibody binding can serve multiple functions, including agglutinizing pathogenic species to enhance clearance and phagocytosis, tagging pathogenic species for opsonization, inducing inflammation, and activating innate immune system responses. In certain cases, antibody binding can also passivate or inactivate a pathogen. Such antibodies are often referred to as neutralizing antibodies, as their binding can limit the pathogenicity and infectivity of a particular pathogen. For example, influenza viruses are thought to be inhibited by influenza hemagglutinin (HA)-specific antibodies which block HA binding to sialic acid to diminish influenza viral envelope-host cell fusion.

As a typical pathogen will display a myriad of antigens (e.g., a virus can display multiple surface proteins with different functions), and each antigen can contain a wide range of potential antibody binding sites (i.e., epitopes), multiple antibodies which target a single antigen can impart drastically different effects. While a typical immune response to pathogenic exposure generates an extensive repertoire of antibodies for each pathogen, the effectiveness of the response can vary greatly based on the specific epitopes which the antibodies target. Accordingly, two pathogen exposure events can confer markedly different immunities to a single pathogen, such that two subjects can have different infection and morbidity risks for the same pathogen. Assessing patient-specific immunity thus requires knowledge of the types of antibodies generated upon exposure to a particular pathogen (e.g., neutralizing vs. non-neutralizing antibodies, in addition to information on the intensity of an immune response overall (e.g., how many antibodies are produced in total).

Nonetheless, distinguishing antibody subpopulations (e.g., neutralizing from non-neutralizing antibodies) has remained a considerable challenge in the field of health diagnostics. As humans possess an average one hundred billion B cells, typical immune responses include complex and multifaceted antibody repertoires with high degrees of paratope and effect variabilities. Further, current in vitro assays for identifying neutralizing antibodies cannot be multiplexed such that multiple variants can be probed in a single assay chamber and these assays are not Clinical Laboratory Improvement Amendments (CLIA) waived, limiting their applicability in point-of-care settings.

Further, live virus neutralization levels are predictive of immune protection against a variety of viral infections, including infection with SARS-CoV-2. A benchmark assay that quantifies the humoral host response against viral infection is the microneutralization assay (MNA), which measures virus-specific neutralizing antibody (NAb) titers and reports neutralization activity as an MN50 value. Unfortunately, this assay takes weeks to complete and requires a BSL-3 lab, making it inconvenient for diagnostic use. Accordingly, new devices and methods are needed to detect neutralizing antibodies in a sample.

### SUMMARY

Disclosed herein is a method for detecting neutralizing antibodies of a viral protein in a biological sample comprising a first set of antibodies and a second set of antibodies, the method comprising: (a) contacting the biological sample with a modified form of the viral protein, thereby binding the first set of antibodies from the biological sample to the modified form of the viral protein, thereby generating a first antibody-protein complex; (b) contacting the biological sample from step (a) with an unmodified viral protein, thereby binding the second set of antibodies from the biological sample to the unmodified viral protein, thereby generating a second antibody-protein complex; (c) separating the first antibody-protein complex from the second antibody-protein complex; and (d) measuring the second antibody-protein complex, thereby detecting the neutralizing antibodies. In some embodiments, the unmodified viral protein is immobilized on a surface. In some embodiments, the second antibody-protein complex remains immobilized on the surface after the separating of step (c). In some embodiments, the separating of step (c) occurs by removing the mixture of the biological sample and the modified form of the viral protein from contacting with the surface. In some embodiments, the unmodified viral protein of step (b) is provided on a microarray. In some embodiments, the modified form of the viral protein of step (a) is provided in a solution comprising the biological sample. In some embodiments, the contacting of step (a) occurs in the solution. In some embodiments, the contacting of step (a) comprises mixing the biological sample with the modified form of the viral protein provided in the solution. In some embodiments, the method further comprises washing the surface prior to the measuring of step (d). In some embodiments, step (d) comprises measuring the amount of the second antibody-protein complex. In some embodiments, step (d) comprises contacting the second antibody-protein complex with a set of labeled anti-IgG antibodies. In some embodiments, the label on the set of anti-IgG comprises a fluorescent label. In some embodiments, the modified form of the viral protein possesses diminished affinity for neutralizing antibodies of the viral protein. In some embodiments, the modified form of the viral protein comprises at least one of a blocked neutralizing antibody epitope and an altered neutralizing antibody epitope. In some embodiments, the modified form of the viral protein comprises an altered neutralizing antibody epitope comprising one or more amino acid variations selected from a mutation, an insertion, a deletion, a chemical modification, a truncation, cleavage, or any combination thereof. In some embodiments, the modified form of the viral protein comprises a blocked neutralizing antibody epitope and a blocking group. In some embodiments, the blocking group is covalently coupled to the blocked neutralizing antibody epitope. In some embodiments, the blocking group is covalently coupled to the blocked neutralizing antibody epitope by a disulfide bond. In some embodiments, the blocking group is covalently coupled to the modified viral protein by a first linker. In some embodiments, the first linker is a polypeptide linker. In some embodiments, the blocking group comprises a receptor protein having an affinity for the viral protein. In some embodiments, the viral protein comprises at least a portion of a coronavirus viral surface protein. In some embodiments, the viral protein comprises at least a portion of a coronavirus viral spike protein. In some embodiments, the viral protein comprises at least a portion of a SARS-CoV-2 surface protein. In some embodiments, the viral protein comprises at least a portion of a receptor binding domain (RBD) of a SARS-CoV-2 spike protein. In some embodiments, the viral protein comprises an amino acid sequence having at least 90%, at least 95%, at least 96%, at least 97%, at least 98%, at least 99%, or 100% sequence identity to amino acids nos. 319 to 541 of SEQ ID NO: 1. In some embodiments, the modified viral protein comprises at least a portion of a SARS CoV-2 surface protein and at least a portion of an angiotensin converting enzyme 2 (ACE2) protein. In some embodiments, the at least a portion of the SARS CoV-2 surface protein comprises an amino acid sequence having at least 90%, at least 95%, at least 96%, at least 97%, at least 98%, at least 99%, or 100% sequence identity to the amino acids nos. 20 to 242 of SEQ ID NO:4, and the at least a portion of the ACE2 protein comprises an amino acid sequence having at least 90%, at least 95%, at least 96%, at least 97%, at least 98%, at least 99%, or 100% sequence identity to amino acids nos. 284 to 880 of SEQ ID NO:4. In some embodiments, the modified viral protein comprises an amino acid sequence having at least 90%, at least 95%, at least 96%, at least 97%, at least 98%, at least 99%, or 100% sequence identity to amino acids nos 20 to 880 of SEQ ID NO:4. In some embodiments, the modified viral protein comprises a sequence having at least 90%, at least 95%, at least 96%, at least 97%, at least 98%, at least 99%, or 100% sequence identity to amino acids nos. 20 to 880 of SEQ ID NO:5. In some embodiments, the SARS-CoV-2 surface protein viral comprises at least a portion of a SARS-CoV-2 variant spike protein. In some embodiments, the SARS-CoV-2 surface protein comprises at least a portion of a receptor binding domain (RBD) of SARS-CoV-2 variant spike protein. In some embodiments, the SARS-CoV-2 surface protein comprises at least a portion of a SARS CoV-2 variant surface protein and at least a portion of an angiotensin converting enzyme 2 (ACE2) protein. In some embodiments, the modified viral protein comprises at least a portion of a SARS CoV-2 variant spike protein and at least a portion of an angiotensin converting enzyme 2 (ACE2) protein, and wherein the at least a portion of the ACE2 protein comprises an amino acid sequence having at least 90%, at least 95%, at least 96%, at least 97%, at least 98%, at least 99%, or 100% sequence identity to amino acids nos. 284 to 880 of SEQ ID NO:4. In some embodiments, the modified viral protein comprises part of a multivalent modified protein complex. In some embodiments, the multivalent protein complex comprises two or more modified viral proteins. In some embodiments, the two or more modified viral proteins are connected covalently or non-covalently via a scaffold or a multimerizing domain. In some embodiments, each of the two or more modified proteins is independently attached to the scaffold or the multimerizing domain via a second linker. In some embodiments, the second linker comprises a polypeptide, a water-soluble oligomer, an alkylene chain, a branched linker, or an organic polymer or any combination thereof. In some embodiments, the second linker comprises a biotin molecule. In some embodiments, the two or more modified viral proteins are connected covalently or non-covalently via a scaffold comprising a protein, a polymer, or any combination thereof. In some embodiments, the scaffold comprises a biotin-binding protein, or a portion thereof. In some embodiments, the scaffold comprises avidin, neutravidin, or streptavidin, or a portion thereof. In some embodiments, the scaffold comprises neutravidin, or a portion thereof. In some embodiments, a biotin molecule connects each of the two or more modified proteins to the scaffold via a noncovalent interaction. In some embodiments, the scaffold comprises a biomolecule. In some embodiments, the scaffold comprises a water-soluble polymer. In some embodiments, the two or more modified viral proteins are connected covalently or non-covalently via a multimerizing domain. In some embodiments, the multimerizing domain comprises a T4 trimerization domain. In some embodiments, each of the two or more modified proteins independently comprises at least a portion of a coronavirus viral surface protein and at least a portion of an angiotensin converting enzyme 2 (ACE2) protein. In some embodiments, the at least a portion of a coronavirus viral surface protein comprises at least a portion of a coronavirus spike protein. In some embodiments, the at least a portion of a coronavirus viral surface protein comprises at least a portion of a receptor binding domain (RBD) of a coronavirus spike protein. In some embodiments, the coronavirus comprises SARS-COV-2. In some embodiments, the at least a portion of the ACE2 protein comprises an amino acid sequence having at least 90%, at least 95%, at least 96%, at least 97%, at least 98%, at least 99%, or 100% sequence identity to amino acids nos. 284 to 880 of SEQ ID NO:4. In some embodiments, each of the two or more modified proteins independently comprises at a sequence having at least 90%, at least 95%, at least 96%, at least 97%, at least 98%, at least 99%, or 100% sequence identity to amino acids nos. 20 to 880 of SEQ ID NO:4. In some embodiments, each of the two or more modified proteins independently comprises a sequence having at least 90%, at least 95%, at least 96%, at least 97%, at least 98%, at least 99%, or 100% sequence identity to amino acids nos. 20 to 880 of SEQ ID NO:5. In some embodiments, the modified viral protein binds to one or more antibodies. In some embodiments, the first set of antibodies comprises a non-neutralizing antibody. In some embodiments, the second set of antibodies comprises neutralizing antibodies with respect to the viral protein. In some embodiments, the biological sample comprises a blood sample taken from a subject. In some embodiments, the subject was previously infected with a viral pathogen that is the origin of the viral protein or received a vaccine for the viral pathogen. In some embodiments, the subject previously contracted SARS-COV-2 or a variant thereof. In some embodiments, the subject previously received a vaccine for SARS-COV-2 or a variant thereof.

Disclosed herein is a multivalent modified protein complex comprising two or more unnatural proteins, wherein each of the two or more unnatural proteins independently comprises (a) a viral protein, (b) a receptor protein having an affinity for the viral protein, and (c) a first linker connecting the viral protein and the receptor protein, wherein each of the two or more unnatural proteins selectively binds to non-neutralizing antibodies of the viral protein over neutralizing antibodies of the viral protein. In some embodiments, the two or more unnatural proteins are connected covalently or non-covalently via a scaffold or a multimerizing domain. In some embodiments, each of the two or more unnatural proteins is independently attached to the scaffold or the multimerizing domain via a second linker. In some embodiments, the second linker comprises a polypeptide, a water-soluble oligomer, an alkylene chain, a branched linker, or an organic polymer or any combination thereof. In some embodiments, the second linker comprises a biotin molecule. In some embodiments, the two or more unnatural proteins are connected covalently or non-covalently via a scaffold comprising a protein, a polymer, or any combination thereof. In some embodiments, the scaffold comprises a biotin-binding protein. In some embodiments, the scaffold comprises avidin, neutravidin, or streptavidin. In some embodiments, the scaffold comprises neutravidin. In some embodiments, a biotin molecule connects each of the two or more unnatural proteins to the scaffold via a noncovalent interaction. In some embodiments, the scaffold comprises a biomolecule. In some embodiments, the scaffold comprises a water-soluble polymer. In some embodiments, the two or more unnatural proteins are connected covalently or non-covalently via a multimerizing domain. In some embodiments, the multimerizing domain comprises a T4 trimerization domain. In some embodiments, each of the two or more unnatural proteins comprises at least 1-order of magnitude greater binding affinity for the non-neutralizing antibodies than for the neutralizing antibodies. In some embodiments, the neutralizing antibodies comprise at least 1-order of magnitude lower binding affinity for the each of the two or more unnatural proteins than for the viral protein in an absence of the receptor protein and the linker. In some embodiments, the viral protein comprises a receptor binding domain (RBD), and wherein the neutralizing antibodies possess affinities for the RBD of the viral protein. In some embodiments, the affinity of the receptor protein for the viral protein is for at least a portion of the RBD of the viral protein. In some embodiments, the viral protein comprises at least a portion of a wild type viral protein. In some embodiments, the viral protein comprises at least a portion of a viral surface protein. In some embodiments, the viral protein comprises at least a portion of a receptor binding domain (RBD) of the viral surface protein. In some embodiments, the viral protein comprises at least a portion of a protein selected from the group consisting of: an influenza A hemagglutinin protein, an influenza A neuraminidase protein, a MERS-CoV spike protein, a SARS-CoV2 spike protein, a zikavirus E protein, a polyomavirus VP1 protein, and an HIV Env protein. In some embodiments, the viral surface protein is a coronavirus viral surface protein. In some embodiments, the coronavirus viral surface protein is a surface protein of SARS-CoV2 or a variant thereof. In some embodiments, the viral protein comprises an amino acid sequence having at least 90%, at least 95%, at least 96%, at least 97%, at least 98%, at least 99%, or 100% sequence identity to amino acids nos. 319 to 541 of SEQ ID NO: 1. In some embodiments, the receptor protein comprises at least a portion of a natural binding target for the viral surface protein. In some embodiments, the receptor protein comprises at least a portion of an angiotensin converting enzyme 2 (ACE2). In some embodiments, the receptor protein comprises a soluble fragment of the angiotensin converting enzyme 2 (ACE2). In some embodiments, the receptor protein comprises an amino acid sequence having at least 90%, at least 95%, at least 96%, at least 97%, at least 98%, at least 99%, or 100% sequence identity to amino acids nos. 18 to 614 of SEQ ID NO:2. In some embodiments, the first linker comprises a polypeptide linker. In some embodiments, the first linker connects the N-terminus of the receptor protein to the C-terminus of the viral protein. In some embodiments, the linker comprises a -GGGGS- pentapeptide motif (SEQ ID NO: 6). In some embodiments, the first linker comprises an endopeptidase recognition sequence. In some embodiments, the viral protein, the first linker, and the receptor protein are encoded by a single expression construct. In some embodiments, the viral protein, the linker, and the receptor protein are encoded by a continuous nucleic acid sequence. In some embodiments, the viral protein of each of the two or more unnatural proteins adopts a substantially similar conformation as the viral protein without the receptor protein and the linker. In some embodiments, each of the two or more unnatural protein independently comprises an amino acid sequence having at least 90%, at least 95%, at least 96%, at least 97%, at least 98%, at least 99%, or 100% sequence identity to the amino acids nos. 20 to 880 of SEQ ID NO:4. In some embodiments, the viral protein and the receptor protein are covalently linked by a disulfide bond. In some embodiments, the disulfide bond is formed between a first cysteine residue on the viral protein and a second cysteine residue on the receptor protein, and wherein at least one of the first cysteine residue and the second cysteine residue is not present in a wild-type sequence of the viral protein or the receptor protein. In some embodiments, the first cysteine residue is not present in the wild-type sequence of the viral protein and the second cysteine residue is not present in the wild-type sequence of the receptor protein. In some embodiments, the disulfide bond is formed between a first thiol on the viral protein and a second thiol on the receptor protein. In some embodiments, at least one of the first thiol and the second thiol is part of an amino acid residue not present in the wild-type sequence of the viral protein or the receptor protein. In some embodiments, the first thiol is part of a first amino acid residue not present in the wild-type sequence of the viral protein and the second thiol is part of a second amino acid residue not present in the wild-type sequence of the receptor protein. In some embodiments, the first amino acid residue and the second amino acid residue are each cysteine. In some embodiments, the first thiol is part of a first cysteine that replaces an amino acid residue corresponding to an amino acid residue between positions 142 and 242 of SEQ ID NO: 4. In some embodiments, the second thiol is part of a second cysteine that replaces an amino acid residue corresponding to an amino acid residue between positions 284 and 340 of SEQ ID NO: 4. In some embodiments, the first cysteine replaces an amino acid residue corresponding to position 196 of SEQ ID NO:4 and the second cysteine residue replaces an amino acid residue corresponding to position 304 of SEQ ID NO:4. In some embodiments, the disulfide bond is an interchain disulfide bond. In some embodiments, each of the two or more unnatural proteins comprises a sequence having at least 90%, at least 95%, at least 96%, at least 97%, at least 98%, at least 99%, or 100% sequence identity to amino acids nos. 20 to 880 of SEQ ID NO:5. In some embodiments, at least one of the viral protein or the receptor protein is a full-length protein that has the same number of amino acids as the wild type sequence of the viral protein or the receptor protein. In some embodiments, at least one of the viral protein or the receptor protein is a protein fragment of the wild type sequence of the viral protein or the receptor protein. In some embodiments, each of the two or more unnatural proteins is substantially inert to human blood proteases. In some embodiments, the affinity of the receptor protein for the viral protein equals to a dissociation constant (KD) of less than 1 μM, 800 nM, 600 nM, 400 nM, 200 nM, 150 nM, 100 nM, 80 nM, 60 nM, 50 nM, 40 nM, or 20 nM.

Disclosed herein is a method for detecting neutralizing antibody levels in a subject, the method comprising: (a) obtaining a biological sample taken from the subject; (b) detecting a concentration of neutralizing antibodies in the sample using a cell-free method; wherein step (b) is completed within one hour. In some embodiments, the method is completed within 30 minutes. In some embodiments, the method is completed within 20 minutes. In some embodiments, the method is completed within 15 minutes. In some embodiments, the neutralizing antibodies are specific for a viral protein. In some embodiments, the subject was previously infected by a viral pathogen that is the origin of the viral protein or received a vaccine against the viral pathogen. In some embodiments, the viral pathogen is a coronavirus. In some embodiments, the viral pathogen is a SARS-COV-2 or a variant thereof. In some embodiments, the agent comprises a modified form of a viral protein. In some embodiments, the agent comprises a multivalent modified protein complex.

Disclosed herein is a method for detecting neutralizing antibody levels in a subject, the method comprising: (a) obtaining a biological sample taken from the subject; (b) adding to the biological sample an agent that selectively binds to non-neutralizing antibodies; and (c) measuring a concentration of neutralizing antibodies in the sample using a cell-free bioassay; wherein step (c) is completed within one hour. In some embodiments, the method is completed within 30 minutes. In some embodiments, the method is completed within 20 minutes. In some embodiments, the method is completed within 15 minutes. In some embodiments, the neutralizing antibodies are specific for a viral protein. In some embodiments, the subject was previously infected by a viral pathogen that is the origin of the viral protein or received a vaccine against the viral pathogen. In some embodiments, the viral pathogen is a coronavirus. In some embodiments, the viral pathogen is a SARS-COV-2 or a variant thereof. In some embodiments, the agent comprises a modified form of a viral protein. In some embodiments, the agent comprises a multivalent modified protein complex.

Disclosed herein is a method for detecting neutralizing antibody levels for two or more viral pathogens in a subject, the method comprising: obtaining a biological sample taken from the subject; adding to the biological sample a first agent that selectively binds to non-neutralizing antibodies against a first viral pathogen; measuring a concentration of neutralizing antibodies against the first viral pathogen in the biological sample using a cell-free bioassay; adding to the biological sample a second agent that selectively binds to non-neutralizing antibodies against a second viral pathogen; and measuring a concentration of neutralizing antibodies against the second viral pathogen in the biological sample using a cell-free bioassay. In some embodiments, the two or more viral pathogens are variants of a viral pathogen. In some embodiments, the viral pathogen is a coronavirus. In some embodiments, the viral pathogen is a SARS-COV-2. In some embodiments, the variants of the viral pathogen comprises Wuhan variant, delta variant, BAI variant, or omicron variant of SARS-CoV-2 or any combination thereof. In some embodiments, the biological sample comprises a blood sample. In some embodiments, the blood sample comprises whole blood. In some embodiments, the blood sample comprises fingerstick blood. In some embodiments, the subject previously received a vaccine against the viral pathogen.

Disclosed herein is a method for distinguishing neutralizing antibody levels for each of two or more variants of a viral pathogen in a subject, the method comprising: obtaining a biological sample taken from the subject; adding to the biological sample a first agent that selectively binds to non-neutralizing antibodies against a first variant of the viral pathogen; measuring a concentration of neutralizing antibodies against the first variant of the viral pathogen in the biological sample using a cell-free bioassay; adding to the biological sample a second agent that selectively binds to non-neutralizing antibodies against second variant of the viral pathogen; measuring a concentration of neutralizing antibodies against the second variant of the viral pathogen in the biological sample using a cell-free bioassay; and comparing the concentrations of neutralizing antibodies against the first variant of the viral pathogen and the concentration of neutralizing antibodies against the second variant of the viral pathogen, thereby distinguishing the neutralizing antibody levels for each of the two or more variants of a viral pathogen in the subject. In some embodiments, the viral pathogen is a coronavirus. In some embodiments, the viral pathogen is a SARS-COV-2. In some embodiments, the variants of the viral pathogen comprises Wuhan variant, delta variant, BAI variant, or omicron variant of SARS-CoV-2 or any combination thereof. In some embodiments, the biological sample comprises a blood sample. In some embodiments, the blood sample comprises whole blood. In some embodiments, the blood sample comprises fingerstick blood. In some embodiments, the subject previously received a vaccine against the viral pathogen.

Disclosed herein is a method for predicting an immune response to a viral pathogen in a subject, the method comprising: (a) obtaining a biological sample taken from the subject; (b) quantifying an observed level of neutralizing antibodies in the sample using a cell-free bioassay procedure comprising: (i) adding to the sample a first agent that selectively binds non-neutralizing antibodies against the viral pathogen thereby generating a pretreated sample; (ii) contacting the pretreated sample with a second agent that binds neutralizing antibodies against the viral pathogen; and (iii) detecting the neutralizing antibodies bound to the second agent; and (c) comparing the observed level of neutralizing antibodies to a predetermined threshold level of neutralizing antibodies. In some embodiments, the predetermined threshold level of neutralizing antibodies is a reference or standard level established by WHO (World Health Organization), Centers for Disease Control and Prevention (CDC), or other public health agencies. In some embodiments, the biological sample comprises a blood sample. In some embodiments, the blood sample comprises whole blood. In some embodiments, the blood sample comprises fingerstick blood. In some embodiments, the subject previously received a vaccine against the viral pathogen.

Disclosed herein is a method for immunizing a subject against a viral pathogen, the method comprising: (a) obtaining a biological sample taken from the subject; (b) quantifying an observed level of neutralizing antibodies in the sample using a cell-free bioassay; (c) comparing the observed level of neutralizing antibodies to a predetermined threshold level of neutralizing antibodies; and (d) if the observed level of neutralizing antibodies in the sample is lower than the predetermined threshold level of neutralizing antibodies, then administering to the subject a vaccine directed against the viral pathogen. In some embodiments, the predetermined threshold level of neutralizing antibodies is a reference or standard level established by WHO (World Health Organization), Centers for Disease Control and Prevention (CDC), or other public health agencies. In some embodiments, the biological sample comprises a blood sample. In some embodiments, the blood sample comprises whole blood. In some embodiments, the blood sample comprises fingerstick blood. In some embodiments, the subject previously received a vaccine against the viral pathogen.

Disclosed herein is a method for treating a subject infected with a viral pathogen, the method comprising: (a) obtaining a biological sample taken from the subject; (b) quantifying an observed level of neutralizing antibodies in the sample using a cell-free bioassay; (c) comparing the observed level of neutralizing antibodies to a predetermined threshold level of neutralizing antibodies; and (d) if the observed level of neutralizing antibodies in the sample is lower than the predetermined threshold level of neutralizing antibodies, then administering to the subject an antiviral therapy directed against the viral pathogen. In some embodiments, the predetermined threshold level of neutralizing antibodies is a reference or standard level established by WHO (World Health Organization), Centers for Disease Control and Prevention (CDC), or other public health agencies. In some embodiments, the biological sample comprises a blood sample. In some embodiments, the blood sample comprises whole blood. In some embodiments, the blood sample comprises fingerstick blood. In some embodiments, the subject previously received a vaccine against the viral pathogen.

Disclosed herein is an unnatural protein comprising: (a) a viral protein, (b) a receptor protein having an affinity for the viral protein, and (c) a linker connecting the viral protein and the receptor protein, wherein the unnatural protein selectively binds to non-neutralizing antibodies of the viral protein over neutralizing antibodies of the viral protein. In some embodiments, the unnatural protein comprises at least 1-order of magnitude greater binding affinity for the non-neutralizing antibodies than for the neutralizing antibodies. In some embodiments, the neutralizing antibodies comprise at least 1-order of magnitude lower binding affinity for the unnatural protein than for the viral protein in an absence of the receptor protein and the linker. In some embodiments, the viral protein comprises a receptor binding domain (RBD), and wherein the neutralizing antibodies possess affinities for the RBD of the viral protein. In some embodiments, the affinity of the receptor protein for the viral protein is for at least a portion of the RBD of the viral protein. In some embodiments, the viral protein comprises at least a portion of a wild type viral protein. In some embodiments, the viral protein comprises at least a portion of a viral surface protein. In some embodiments, the viral protein comprises at least a portion of a receptor binding domain of the viral surface protein. In some embodiments, the viral protein comprises at least a portion of a protein selected from the group consisting of an influenza A hemagglutinin protein, an influenza A neuraminidase protein, a MERS-CoV spike protein, a SARS-CoV2 spike protein, a zikavirus E protein, a polyomavirus VP1 protein, and an HIV Env protein. In some embodiments, the viral surface protein is a coronavirus viral surface protein. In some embodiments, the coronavirus viral surface protein is a surface protein of SARS-CoV2. In some embodiments, the viral protein comprises an amino acid sequence having at least 90%, at least 95%, at least 96%, at least 97%, at least 98%, at least 99%, or 100% sequence identity to amino acids nos. 319 to 541 of SEQ ID NO: 1. In some embodiments, the receptor protein comprises at least a portion of a natural binding target for the viral surface protein. In some embodiments, the receptor protein comprises at least a portion of an angiotensin converting enzyme 2 (ACE2). In some embodiments, the receptor protein comprises a soluble fragment of the angiotensin converting enzyme 2 (ACE2). In some embodiments, the receptor protein comprises an amino acid sequence having at least 90%, at least 95%, at least 96%, at least 97%, at least 98%, at least 99%, or 100% sequence identity to amino acids nos. 18 to 614 of SEQ ID NO:2. In some embodiments, the linker comprises a polypeptide linker. In some embodiments, the linker connects the N-terminus of the receptor protein to the C-terminus of the viral protein. In some embodiments, the linker comprises a -GGGGS- pentapeptide motif (SEQ ID NO: 6). In some embodiments, the linker comprises an endopeptidase recognition sequence. In some embodiments, the viral protein, the linker, and the receptor protein are encoded by a single expression construct. In some embodiments, the viral protein, the linker, and the receptor protein are encoded by a continuous nucleic acid sequence. In some embodiments, the viral protein of the unnatural protein adopts a substantially similar conformation as the viral protein without the receptor protein and the linker. In some embodiments, the unnatural protein comprises an amino acid sequence having at least 90%, at least 95%, at least 96%, at least 97%, at least 98%, at least 99%, or 100% sequence identity to the amino acids nos. 20 to 880 of SEQ ID NO:4. In some embodiments, the viral protein and the receptor protein are covalently linked by a disulfide bond. In some embodiments, the disulfide bond is formed between a first cysteine residue on the viral protein and a second cysteine residue on the receptor protein, and wherein at least one of the first cysteine residue and the second cysteine residue is not present in a wild-type sequence of the viral protein or the receptor protein. In some embodiments, the first cysteine residue is not present in the wild-type sequence of the viral protein and the second cysteine residue is not present in the wild-type sequence of the receptor protein. In some embodiments, the unnatural protein is immobilized on a surface. In some embodiments, the unnatural protein is coupled to the surface by a biotin binding agent. In some embodiments, the unnatural protein is randomly oriented on the surface. In some embodiments, the unnatural protein is substantially inert to human blood proteases. In some embodiments, the affinity of the receptor protein for the viral protein is at least about 1 μM. In some embodiments, the affinity of the receptor protein for the viral protein equals to a dissociation constant (KD) of less than 1 μM, 800 nM, 600 nM, 400 nM, 200 nM, 150 nM, 100 nM, 80 nM, 60 nM, 50 nM, 40 nM, or 20 nM.

Disclosed herein is an unnatural protein comprising a viral protein and a receptor protein having a binding affinity for the viral protein, wherein the viral protein is covalently linked to the receptor protein via a disulfide bond between a first thiol on the viral protein and a second thiol on the receptor protein. In some embodiments, at least one of the first thiol and the second thiol is part of an amino acid residue not present in a wild-type sequence of the viral protein or the receptor protein. In some embodiments, the first thiol is part of a first amino acid residue not present in a wild-type sequence of the viral protein and the second thiol is part of a second amino acid residue not present in a wild-type sequence of the receptor protein. In some embodiments, the first amino acid residue and the second amino acid residue are each cysteine. In some embodiments, the viral protein comprises at least a portion of a receptor binding domain of a viral surface protein. In some embodiments, the viral protein comprises a viral protein selected from the group consisting of: an influenza A hemagglutinin protein, an influenza A neuraminidase protein, a MERS-CoV spike protein, a SARS-CoV2 spike protein, a zikavirus E protein, a polyomavirus VP1 protein, and an HIV Env protein. In some embodiments, the receptor protein comprises a natural host receptor protein for the viral protein. In some embodiments, the viral protein comprises at least a portion of a coronavirus viral surface protein. In some embodiments, the viral protein comprises at least a portion of a SARS-CoV2 viral surface protein. In some embodiments, the receptor protein comprises at least a portion of an angiotensin converting enzyme 2 (ACE2). In some embodiments, the first thiol is part of a first cysteine that replaces an amino acid residue corresponding to an amino acid residue between positions 142 and 242 of SEQ ID NO: 4. In some embodiments, the second thiol is part of a second cysteine that replaces an amino acid residue corresponding to an amino acid residue between positions 284 and 340 of SEQ ID NO: 4. In some embodiments, the first cysteine replaces an amino acid residue corresponding to position 196 of SEQ ID NO:4 and the second cysteine residue replaces an amino acid residue corresponding to position 304 of SEQ ID NO:4. In some embodiments, the disulfide bond is an interchain disulfide bond. In some embodiments, the N-terminus of the receptor protein is connected to the C-terminus of the viral protein by a linker. In some embodiments, the linker is a polypeptide linker, and the disulfide bond is an intrachain disulfide bond. In some embodiments, at least one of the viral protein or the receptor protein is a full-length protein that has the same number of amino acids as the wild type sequence of the viral protein or the receptor protein. In some embodiments, at least one of the viral protein or the receptor protein is a protein fragment of the wild type sequence of the viral protein or the receptor protein. In some embodiments, the unnatural protein comprises an amino acid sequence having at least 90%, at least 95%, at least 96%, at least 97%, at least 98%, at least 99%, or 100% sequence identity to amino acid nos. 20 to 242 of SEQ ID NO:5. In some embodiments, the unnatural protein comprises an amino acid sequence having at least 90%, at least 95%, at least 96%, at least 97%, at least 98%, at least 99%, or 100% sequence identity to amino acid nos. 284 to 880 of SEQ ID NO:5. In some embodiments, the unnatural protein is immobilized on a surface.

Disclosed herein is a nucleic acid molecule having a sequence encoding the two or more unnatural proteins described herein or the unnatural protein described herein.

Disclosed herein is a vector comprising the nucleic acid molecule described herein.

Disclosed herein is a host cell comprising the nucleic acid molecule described herein or the vector described herein.

Disclosed herein is a method, comprising culturing the host cell described herein under conditions for production of the two or more unnatural proteins described herein or the unnatural protein described herein.

Disclosed herein is a testing kit comprising the multivalent modified protein complex described herein or the unnatural protein described herein and instructions specified for using the testing kit to detect neutralizing antibodies in a biological sample taken from a subject.

Disclosed herein is a method for detecting neutralizing antibodies of a viral protein in a biological sample, the method comprising: (a) contacting the biological sample with the viral protein, thereby binding a first set of antibodies from the biological sample to the viral protein; (b) measuring the first set of antibodies bound to the viral protein; (c) contacting the biological sample to a modified form of the viral protein, thereby binding a second set of antibodies from the biological sample to the modified form of the viral protein; (d) measuring the second set of antibodies bound to the modified form of the viral protein; and (e) determining a difference between the measurement of the first set of antibodies from step (b) and the measurement of the second set of antibodies from step (d), thereby detecting the neutralizing antibodies in the biological sample. In some embodiments, the modified form of the viral protein possesses diminished affinity for neutralizing antibodies of the viral protein. In some embodiments, the difference corresponds to a difference in an abundance of the first set of antibodies and an abundance of the second set of antibodies. In some embodiments, the difference corresponds to a difference in neutralizing antibody abundance between the first set of antibodies and the second set of antibodies. In some embodiments, the modified form of the viral protein comprises at least one of a blocked neutralizing antibody epitope and an altered neutralizing antibody epitope. In some embodiments, the modified form of the viral protein comprises an altered neutralizing antibody epitope comprising a mutation, a chemical modification, cleavage, or any combination thereof. In some embodiments, the modified form of the viral protein comprises a blocked neutralizing antibody epitope and a blocking group. In some embodiments, the blocking group is covalently coupled to the blocked neutralizing antibody epitope. In some embodiments, the blocking group is covalently coupled to the blocked neutralizing antibody epitope by a disulfide bond. In some embodiments, the blocking group is covalently coupled to the modified viral protein by a polypeptide linker. In some embodiments, the modified viral protein is expressed as a single expression construct comprising the blocked neutralizing antibody epitope, the polypeptide linker, and the blocking group. In some embodiments, the blocking group comprises a receptor protein of the viral protein. In some embodiments, the viral protein comprises a fragment of a full-length viral protein. In some embodiments, the viral protein comprises at least a portion of a coronavirus viral surface protein. In some embodiments, the viral protein comprises at least a portion of a SARS-CoV-2 surface protein. In some embodiments, the viral protein comprises an amino acid sequence having at least 90%, at least 95%, at least 96%, at least 97%, at least 98%, at least 99%, or 100% sequence identity to amino acids nos. 319 to 541 of SEQ ID NO: 1. In some embodiments, the modified viral protein comprises at least a portion of a SARS CoV-2 surface protein and at least a portion of an angiotensin converting enzyme 2 (ACE2) protein. In some embodiments, the at least a portion of the SARS CoV-2 surface protein comprises an amino acid sequence having at least 90%, at least 95%, at least 96%, at least 97%, at least 98%, at least 99%, or 100% sequence identity to amino acids 20 to 242 of SEQ ID NO:4, and the at least a portion of the ACE2 protein comprises an amino acid sequence having at least 90%, at least 95%, at least 96%, at least 97%, at least 98%, at least 99%, or 100% sequence identity to amino acids 284 to 880 of SEQ ID NO:4. In some embodiments, the viral protein of (a) and the modified form of the viral protein of (c) are provided on a microarray. In some embodiments, step (b) comprises contacting the first set of antibodies and a first set of labeled anti-IgG antibodies and step (d) comprises contacting the second set of antibodies with a second set of labeled anti-IgG antibodies. In some embodiments, the label on either or both of the first set of anti-IgG antibodies and the second set of anti-IgG antibodies comprises a fluorescent label. In some embodiments, step (b) and step (d) occur simultaneously. In some embodiments, the modified viral protein comprises a sequence having at least 90% sequence identity to amino acids nos 20 to 880 of SEQ ID NO:4. In some embodiments, the modified viral protein comprises a sequence having at least 90% sequence identity to amino acids nos 20 to 880 of SEQ ID NO:5. In some embodiments, the first set of antibodies comprises neutralizing antibodies with respect to the viral protein. In some embodiments, the second set of antibodies comprises non-neutralizing antibodies with respect to the viral protein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “figure” and “FIG.” herein), of which:

FIG. 1A depicts the entry of enveloped virus 111 into host cell 101 through membrane fusion 112.

FIG. 1B depicts the entry of nonenveloped virus 161 into host cell 101 through endocytosis 162.

FIG. 2A shows the RBD of the coronavirus spike(S) protein binding to the ACE2 protein on the surface of a host cell, facilitating entry of the coronavirus to the host cell.

FIG. 2B depicts a coronavirus membrane with the spike surface glycoprotein and other structural proteins (panel A), the binding of the spike protein trimer to the ACE2 protein on a host cell (panel B); and the RBD subdomain of the spike protein monomer (panel C).

FIG. 3A shows unnatural proteins 310 and 320 of the present disclosure, each comprising a viral protein 301, receptor protein 304, and linker 303.

FIG. 3B depicts a plurality of unnatural proteins 320 immobilized on surface 330.

FIG. 3C shows immobilization of a plurality of unnatural proteins 320 to surface 330 via the interaction between biotin 306 and biotin-binding motif 307.

FIG. 4A shows the structure of a stapled unnatural protein 401 comprising the RBD of the SARS-CoV-2 spike protein 411, the ectodomain of the ACE2 receptor protein 412, and linker 413. Disulfide bond 414 covalently links the RBD of the SARS-CoV-2 spike protein 411 to the ectodomain of the ACE2 receptor protein 412.

FIG. 4B shows a closer view of the interface between the RBD of the SARS-CoV-2 spike protein 411 and the ectodomain of the ACE2 receptor protein 412, highlighting the positioning of disulfide bond 414.

FIG. 5A shows a plurality of unnatural proteins 501 of the present disclosure immobilized on surface 504 and binding selectively to non-neutralizing antibodies 502.

FIG. 5B shows a plurality of viral proteins 505 of the present disclosure immobilized on surface 504 and binding to both non-neutralizing antibodies 502 and neutralizing antibodies 506.

FIG. 5C provides a sample calibration curve demonstrating the cut-off for detecting neutralizing antibodies in a biological sample.

FIG. 6A and FIG. 6B provide alternative embodiments of methods of the present disclosure for the detection of neutralizing antibodies. FIG. 6A depicts a substrate comprising a receptor for a viral protein. FIG. 6B depicts a substrate bound to a viral protein.

FIG. 6C provides a sample calibration curve demonstrating the cut-off for detecting neutralizing antibodies in a biological sample.

FIGS. 7A to 7D provide an alternative method for detecting and/or quantifying neutralizing antibodies in a biological sample. FIG. 7A depicts a sample comprising a mixture of a first set of antibodies and a second set of antibodies. FIG. 7B depicts a first antibody-protein complex in solution and a second antibody-protein complex immobilized on a surface. FIG. 7C depicts the first set of antibodies separated from the second set of antibodies. FIG. 7D depicts contacting the second antibody-protein complex with a labeled anti-IgG antibody.

FIG. 8A provides a schematic of a multivalent fusion protein complex of the present disclosure.

FIG. 8B provides an exemplary structure of a multivalent fusion protein complex of the present disclosure.

FIG. 9A provides a block diagram of one unnatural protein embodiment of the present disclosure. Figure discloses SEQ ID NO: 8.

FIG. 9B provides a block diagram of one stapled unnatural protein embodiment of the present disclosure, with points of cysteine mutations identified.

FIG. 10 demonstrates comparable binding between commercial SARS-CoV-2 S-protein RBD and the SARS-CoV-2 S-protein RBD standard used in the examples of the present disclosure.

FIG. 11 shows the binding of five different antibodies to immobilized SARS-CoV-2 S-protein RBD from the original strain and the delta variant.

FIG. 12 shows binding of three different antibodies (non-neutralizing antibody 1106 and neutralizing antibodies 107 and E6) to immobilized SARS-CoV-2 S-protein RBD and an immobilized fusion protein of the present disclosure.

FIG. 13 shows binding of three different antibodies (non-neutralizing antibody 1106 and neutralizing antibodies 107 and E6) to immobilized SARS-CoV-2 S-protein RBD and an immobilized stapled fusion protein of the present disclosure.

FIG. 14A shows binding of six different anti-spike-RBD non-neutralizing antibodies (a: 1106, b: 5305, c: 5308, d: S1N-M130, e: A02038, f: 91349) for two different RBD-ACE2 fusion proteins (B-3A and B-M3D4).

FIG. 14B shows binding of four different anti-spike-RBD neutralizing antibodies (g: 107, h: E6, i: 91383, j: 91361) for two different RBD-ACE2 fusion proteins (B-3A and B-M3D4).

FIG. 15A provides BAU/ml and IU/ml values of WHO standard and Reference Panel. BAU: binding antibody units, IU: International units.

FIG. 15B provides ELISA results using in-house RBD protein (B-M5B) converted to BAU/ml.

FIG. 15C provides ELISA results using in-house RBD-ACE2 fusion proteins (B-3A and B-M3D4) converted to IU/ml.

FIG. 16 demonstrates greater NAb antibody detection signal using a multivalent fusion protein complex of the present disclosure.

FIG. 17 depicts the detection of neutralizing antibodies using a method of the present disclosure on four samples: (1) a sample from a single vaccinated individual, (2) a pooled sample of convalescent plasma from several individuals previously infected with SARS-CoV2; (3) a pooled sample from several vaccinated individuals; and (4) a sample from a single vaccinated individual who had recently been infected with SARS-CoV2.

FIG. 18 provides a table summarizing the medical history of the individuals that provided samples analyzed in FIGS. 18A-C.

FIG. 18A depicts the intensity of NAb signal directed at the SARS RBM from the samples described in FIG. 18 and demonstrates that nearly all samples derived from vaccinated or infected individuals provided a positive NAb signal in a method of the present disclosure.

FIG. 18B depicts the intensity of NAb signal directed at nucleoprotein (“NP”) from a subset of samples described in FIG. 18 and demonstrates that all samples derived from infected individuals provided a positive NAb signal in a method of the present disclosure.

FIG. 18C demonstrates a strong correlation between a method the present disclosure and the live virus microneutralization values (MN₅₀) as determined by the microneutralization assay for the measurement of neutralizing antibodies.

FIG. 18D further demonstrates a strong correlation between a method the present disclosure and the live virus microneutralization values (MN₅₀) as determined by the microneutralization assay for the measurement of neutralizing antibodies, providing the R² fit between different mathematical regressions of the data.

FIG. 19A provides NAb values determined by a method of the present disclosure for ten different individuals vaccinated with the Moderna SARS-CoV2 mRNA vaccine. Each plot represents samples taken at 8 different time points between 0 and 300 days post-vaccination.

FIG. 19B provides NAb values for each of the 10 individuals prior to either reinfection or booster shot using the Moderna SARS-CoV2 mRNA vaccine, demonstrating a model for the reduction in NAb levels over time.

FIG. 20 depicts the NAb values detected using a method of the present disclosure for NAbs directed at (1) the original Wuhan SARS-CoV2 strain, (2) the omicron BA.1 variant, and (3) the omicron BA.2 variant, showing that vaccinated and/or patients previously infected with the Wuhan strain exhibit significantly lower NAb levels directed at the two omicron variants.

### DETAILED DESCRIPTION

Responsive to the need for neutralizing and non-neutralizing antibody discriminating methods, aspects of the present disclosure provide systems, compositions, and methods for detecting antibody subpopulations from biological samples. In some embodiments, systems, compositions, and methods disclosed herein provided increased sensitivity for an antibody subpopulation from biological samples. In some embodiments, the present disclosure provides systems, compositions, and methods for detecting neutralizing antibodies for an antigen found on a pathogen (e.g., a viral pathogen, bacterial pathogen, fungal pathogen, etc.).

ELISA based methods can be used to detect antibodies that bind to a viral protein. Incubation of a sample containing a collection of antibodies with a viral protein results in binding and detection of all antibodies that bind to the viral protein. This collection of binding antibodies will comprise (a) neutralizing antibodies that bind to the portion of the viral protein involved in binding to the host cell surface proteins; and (b) non-neutralizing antibodies that bind to other portions of the viral protein that are not involved in binding to host cell surface proteins (i.e., receptor binding portion). Identifying neutralizing antibodies requires separating (a) neutralizing antibodies from (b) non-neutralizing antibodies.

Detection of neutralizing antibodies is achieved by a background subtraction method: a first signal corresponding to a population of non-neutralizing antibodies is subtracted from a second signal corresponding to a population of both neutralizing and non-neutralizing antibodies. The first signal (derived only from non-neutralizing antibodies) can be obtained using combinations of a viral protein and the receptor for the viral protein. The viral protein and its cognate receptor bind to each other forming a viral protein/receptor complex. The viral protein/receptor complex blocks the receptor binding portion of the viral protein so that neutralizing antibodies from the sample cannot be detected. Thus, the first signal is derived from exposure of the sample to the viral protein/receptor complex and corresponds only to non-neutralizing antibodies. The second signal is generated by exposure of the sample to the viral protein in the absence of the cognate receptor, allowing for detection of both neutralizing and non-neutralizing antibodies.

For example, a microarray co-spotted with both (1) a viral protein, and (2) a viral protein/receptor complex, probed with a sample comprising a neutralizing monoclonal IgG antibody, could, for example, demonstrate a decrease in antibody binding signal for the viral protein/receptor complex compared to the viral protein. The difference between the intensity of these signals (i.e., following background subtraction) corresponds to the neutralizing antibodies in the sample. In contrast, the same microarray probed with a sample lacking neutralizing monoclonal IgG antibody would result in the same signal from both spots. Background subtraction in this situation would result in little to no signal, indicating a lack of neutralizing antibodies in the sample.

However, simply co-spotting the viral protein with the cognate host cell receptor likely result in a relatively low detection sensitivity for neutralizing antibodies due to dissociation of the viral protein/receptor complex. For example, if a significant proportion of the viral protein/receptor complex dissociates during the assay procedure, the neutralizing antibodies can bind to the viral protein on the spots associated with the viral protein/receptor complex. This will cause the signal from the spot with the viral protein/receptor complex (i.e., the background signal that is supposed to correspond to only non-neutralizing antibodies) to be artificially high because it includes both neutralizing and non-neutralizing antibodies. Subtraction of this artificially high background signal (which includes signal from some neutralizing antibodies) from the signal from the spot with only the viral protein (i.e., corresponding to both neutralizing and non-neutralizing antibodies) will result in a decreased final signal (which is supposed to correspond to the neutralizing antibody population). Thus, the sensitivity of the assay for neutralizing antibodies will be diminished by the dissociation of the viral protein/receptor complex. The resulting background noise in the data caused by dissociation of the significantly will thereby decrease the utility of such assays for the detection of neutralizing antibodies.

The process of detecting neutralizing antibodies comprises a number of steps in which dissociation of the viral protein/receptor complex can occur. For example, when significant dilution of the viral protein/receptor complex occurs, the complex is prone to dissociation. Significant dilution occurs throughout the process of detecting neutralizing antibodies, including during microarray incubation in plasma, secondary antibody addition, and washes between each step. During each of these steps, some amount of viral protein/receptor complex is likely dissociated Thus, during the process of neutralizing antibody detection, there is significant risk of dissociation of the viral protein/receptor complex. The resulting background noise in the data significantly limits the sensitivity of such assays for detecting neutralizing antibodies and limits their utility.

To address these issues, the present disclosure provides devices and methods for enhancing the sensitivity of neutralizing antibody detection. In some embodiments, these devices and methods increase the sensitivity of neutralizing antibody detection by reducing the background noise caused by dissociation of the viral protein/receptor complex. In some embodiments, the increased sensitivity is achieved by reducing or eliminating dissociation of the viral protein/receptor complex. In some embodiments, the methods and devices utilize a covalent protein complex comprising a viral protein that is covalently coupled to a receptor protein comprising an affinity for the viral protein. Covalent coupling of the viral protein to the receptor protein reduces or eliminates the dissociation of the viral protein from the receptor protein. The covalent protein complex selectively binds to non-neutralizing antibodies of the viral protein over neutralizing antibodies of the viral protein. The increased stability of the covalent protein complex reduces or prevents dissociation of the viral protein from the receptor protein, providing an improved signal corresponding to non-neutralizing antibodies. In some embodiments, the covalent protein complex is an unnatural protein comprising the viral protein, the receptor protein and a linker connecting the viral protein to the receptor. In some embodiments, the covalent protein complex is a fusion protein comprising the viral protein, the receptor protein and a linker connecting the viral protein to the receptor. In some embodiments, the covalent protein complex comprises a covalent bond between a first amino acid residue on the viral protein and a second amino acid residue on the receptor protein. In some embodiments, the covalent bond between the first amino acid residue and the second amino acid residue is a disulfide bond.

In this way, embodiments of the present disclosure ensure that the relative local concentrations of the two proteins (e.g., viral protein and receptor protein) remains very high (for example, in the few micromolar range given with a short flexible linker) with no change resulting from dilution. By covalently linking the two proteins, the viral protein and the receptor protein will remain complexed through the normal interaction site. As demonstrated herein, covalent protein complexes show a large differential when probing with a neutralizing antibody. On the other hand, when probing in ELISA assays with a non-neutralizing antibody, the signals from the viral protein and the covalent protein complex are essentially the same. Thus, the embodiments of the present disclosure provide enhanced sensitivity for the detection of neutralizing antibodies.

Devices, methods, and compositions of the present disclosure can be used for the detection of neutralizing antibodies directed antigens from many different pathogens. Although many embodiments and examples of the present disclosure are directed at viral proteins, the present disclosure is generalizable and can be used for the detection of neutralizing antibodies directed at non-viral antigens. In some embodiments, the pathogen is a bacterium, a virus, a fungus, or a protozoon. In some embodiments, the pathogen is a bacterium or a virus. In some embodiments, the pathogen is a virus.

The present disclosure further provides methods for distinguishing neutralizing and non-neutralizing antibodies of a pathogenic biomolecule. In some embodiments, the pathogenic biomolecule is a saccharide, a nucleic acid, a lipid, a peptide, a protein, or any combination thereof. In some cases, the pathogenic biomolecule is a protein. In some embodiments, the pathogenic biomolecule is a portion of a protein, such as a soluble domain of a transmembrane viral protein. In some cases, the pathogenic biomolecule is a viral biomolecule. In some embodiments, the pathogenic biomolecule is a viral protein. In some embodiments, the viral biomolecule is a viral surface biomolecule, such as for example a viral surface protein. Many viral surface proteins exist, some of which are involved (directly or indirectly) in binding of the virus to a host cell and internalization of the virus into the host cell.

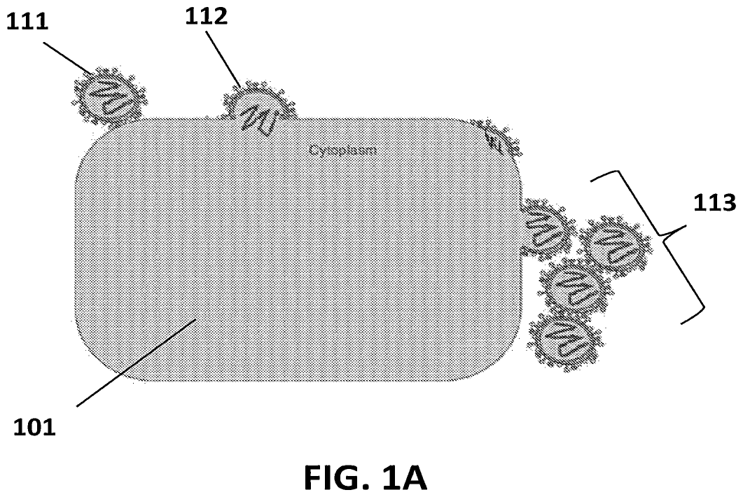

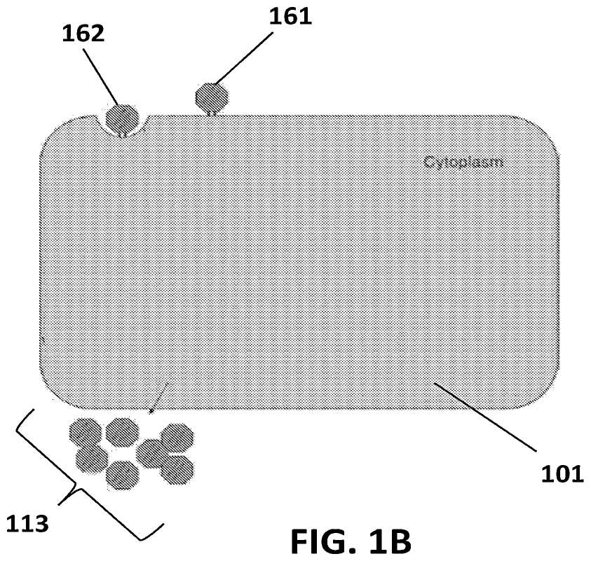

In some embodiments, the viral surface protein is an enveloped virus surface protein. Enveloped viruses (including. e.g., human immunodeficiency virus (HIV), Ebola virus, and SARS-CoV2) utilize surface glycoproteins that protrude from the lipoprotein membrane envelope to bind to target host cells. As shown in FIG. 1A, binding of the viral surface protein on enveloped virus 111 to the cognate receptor on host cell 101 leads to a membrane fusion process 112 in which the host cell membrane is punctured and fuses with the unfolding viral envelope. As the viral envelope merges with the host cell membrane, the virus is internalized. After viral replication, viral budding and release 103 occurs. Some enveloped viruses comprise more than one viral surface protein. For example, coronaviruses typically comprise three surface proteins: Envelope protein (E) Membrane protein (M), and Spike glycoprotein(S).

In some embodiments, the viral surface protein is a nonenveloped virus surface protein. Nonenveloped viruses (including, e.g., polyomavirus, rhinovirus, adenovirus) bind to target host cells using viral surface protein that comprises the protein capsid (i.e., the protein shell that encapsulates the genetic material of the virus). As shown in FIG. 1B, binding of the surface protein on nonenveloped virus 161 to the cognate receptor on host cell 101 leads to virus internalization via endocytosis 162 by the host cell. After viral replication, viral budding and release 163 occurs.

Discussed below are several viral families for which the present disclosure provides a method of detecting neutralizing antibodies. The examples and embodiments below are exemplary and do not limit the general applicability of the present disclosure.

### Coronavirus Diseases

Coronaviridae, a family of related, primarily positive-strand RNA viruses, have attracted considerable attention in 21^{st} century medicine. Diseases associated with the Coronaviridae family include avian infectious bronchitis, Middle East respiratory syndrome (MERS), severe acute respiratory syndrome (SARS), certain forms of transmissible gastroenteritis, certain forms of pneumonia, murine encephalitis, and Coronavirus disease 2019 (COVID-19). Members of this family are commonly (although not exclusively) distinguished by a ‘stellar corona’ likeness punctuated by petal or spike shaped viral surface proteins which often are involved in receptor recognition and viral attachment to human cells, can impart a range of infectivity. Many of these surface proteins are relatively large (e.g., apparent diameters greater than 10 nm), and comprise distinct receptor binding domains (RBDs) responsible for receptor binding activity. In some cases, neutralizing antibodies of a Coronaviridae viruses target this receptor binding domain, which can comprise only a small fraction of the dimensions of the surface protein.

The most abundant protein on the surface of coronaviruses is the spike protein(S). The S protein is divided into 2 domains, S2 and S1. S2 is proximal to the virus membrane surface and contains a transmembrane subdomain that anchors it to the viral membrane. The S1 domain contains a subdomain, RBD (receptor binding domain), which is involved in the attachment of the virus to the host cell. The part of the RBD which physically contacts the host receptor is referred to as the receptor binding motif (RBM). In the case of Severe Acute Respiratory Syndrome Coronavirus (SARS-CoV) and SARS-CoV2 the RBM makes contact with the cell surface receptor ACE2 (Angiotensin I Converting Enzyme 2)

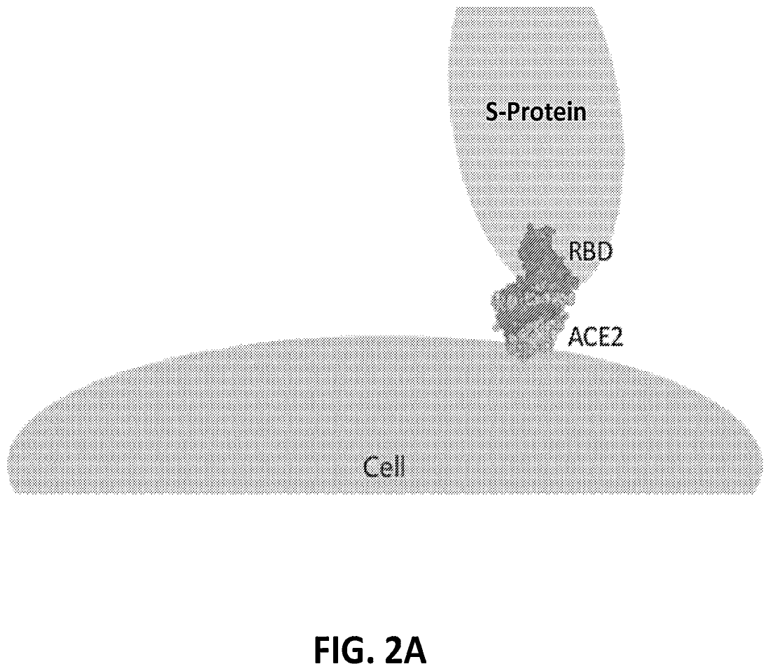

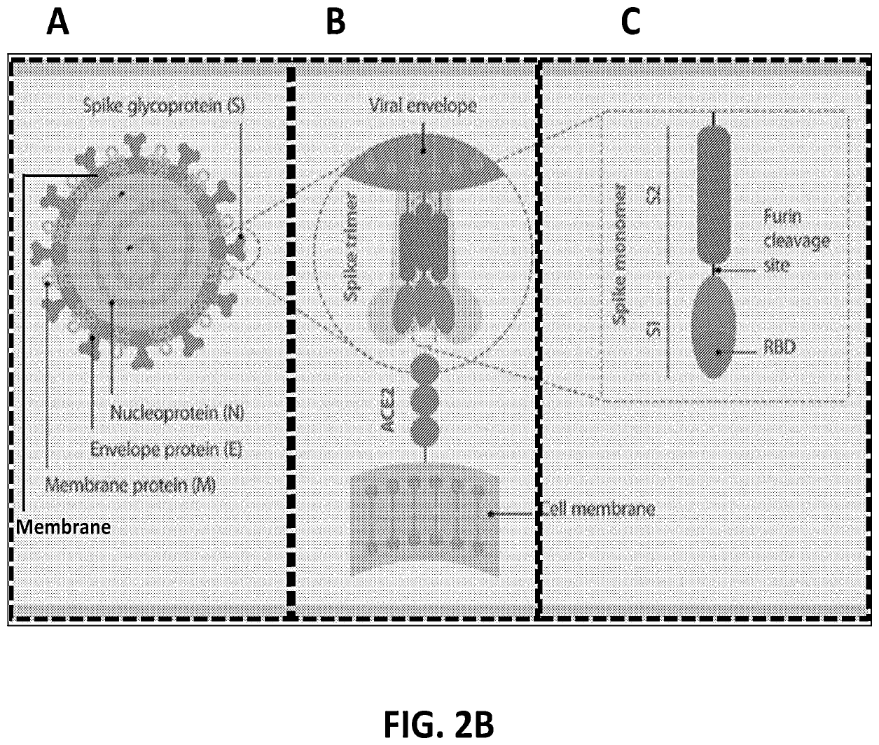

FIG. 2A provides a schematic of a COVID-19 Spike-protein, or S-protein (a viral surface protein) bound to ACE2 of a cell membrane. As depicted in the schematic, a single domain of the S-protein, a surface of the receptor binding domain (RBD), binds to ACE2 on the surface of the cell. Accordingly, an antibody which targets a portion of the S-protein other than its RBD may not directly block ACE2 binding.

FIG. 2B overviews COVID-19 (panel A), S-protein trimer (panel B), and S-protein monomer structure. COVID-19 can comprise a core with single-stranded RNA nucleoproteins (N) encapsulated by a membrane comprising lipids and a number of surface proteins, including envelope (E) protein, membrane (M) protein, and the Spike glycoprotein (S). As depicted in panel B, the spike glycoprotein can be present as a trimer (e.g., a homotrimer) comprised of three identical subunits projecting out of the COVID-19 viral envelope. As depicted in panel C, a monomeric S-protein can comprise an S1 domain coupled to a membrane bound S2 domain by a furin cleavage site. A portion of the S1 domain can be comprise of a receptor binding domain which can comprise a binding affinity for an ACE2 protein.

It has been found that the preponderance of neutralizing antibodies generated by a SAR2-CoV2 infection interfere with the RBD/ACE2 interaction by binding the RBM. Current SARS-CoV2 serology assays contains a microarray of S. RBD, and Nucleoprotein (NP) proteins. In order to extend the assay to probing for neutralizing antibodies on the microarray, RBD complexed with ACE2 [FIG. 2] has been spotted on the array at the same molar concentration as RBD alone.

### Influenza Virus Diseases

Influenza, commonly known as “the flu”, is an infectious disease caused by influenza viruses. Symptoms range from mild to severe, and the CDC estimates that flu has resulted in 9 million-41 million illnesses, 140,000-710,000 hospitalizations and 12,000-52,000 deaths annually between 2010 and 2020. There are four general types of influenza virus, known as influenza viruses A, B, C, and D. Influenza A virus (IAV) is primarily found in aquatic birds, but IAV is also widespread in various mammals, including humans and pigs. For example, the H1N1 influenza is a subtype of IAV, which led to an outbreak in 2009-2010. Influenza B virus (IBV) and Influenza C virus (ICV) primarily infect humans, and Influenza D virus (IDV) is primarily found in cattle and pigs. IAV and IBV circulate in humans and cause seasonal epidemics, and ICV causes a mild infection, primarily in children. Influenza is an enveloped virus with a lipid-bilayer membrane and three types of integral membrane proteins, Hemagglutinin (HA), Neuraminidase (NA) and the M2 protein. Mutations in HA and NA result in dozens of viral subtypes (e.g., H1N1, H5N1, H7N9, etc.).

The influenza virus enters the host cell by binding of its envelope proteins to glycoproteins on the target host cell. The HA and NA glycoproteins bind to the sialic acid found on glycoproteins or glycolipid receptors of the host cell. The HA protein is a trimeric envelope glycoprotein consisting of a globular receptor-binding domain (HA-RBD) that is inserted into a membrane fusion-mediating stalk domain. Binding of HA and/or NA to glycoproteins on the host cell lead to endocytosis of the virus into the host cell. Several additional host cell proteins have been implicated in binding to NA and endocytosis of various influenza virus subtypes, including nucleolin for H7N9, H3N2, PR8 and H5N1, epidermal growth factor receptor (EGFR) for H7N9 and PR8; and voltage-dependent calcium channel CaV1.2 for H1N1, H3N2 and PR8. Many neutralizing antibodies against influenza bind to the RBD of HA and, to a lesser extent, the RBD of NA.

### Polyomavirus Diseases

Polyomaviridae is a family of viruses whose natural hosts are primarily mammals and birds. There are six recognized genera and 117 species in the Polyomaviridae family. Fourteen species are known to infect humans, while others, such as Simian Virus 40, have been identified in humans to a lesser extent. Although a majority of polyomavirus infections are asymptomatic, diseases caused by human polyomavirus infections are most common among immunocompromised people. Such diseases include BK virus is associated with nephropathy in renal transplant and non-renal solid organ transplant patients, JC virus infection is associated with progressive multifocal leukoencephalopathy, and Merkel cell virus (MCV) is associated with Merkel cell cancer.

VP1 is the major structural component of the polyomavirus icosahedral capsid, which contains three proteins (VP1, VP2, and VP3). The VP1 protein is responsible for initiating the process of infecting host cells by binding to sialic acids in glycans, including some gangliosides, on the surface of the host cell. Canonically, VP1 interacts specifically with a (2,3)-linked and a (2,6)-linked sialic acids, leading to internalization of the virus by the host cell. Recent evidence has implicated mutations in the receptor binding region of VP1 in polyomavirus variants that escape the immune system, demonstrating that neutralizing antibodies recognize antigenic epitopes on the receptor-binding sites of VP1.

### Other Pathogens

The compositions, methods, and devices of the present disclosure can also be used to detect neutralizing antibodies for any number of pathogens. Such pathogens can include, for example, bacterial, viral, and fungal pathogens. Exemplary viral pathogens include, for example, human immunodeficiency virus, a hepatitis virus, a zikavirus, a rhinovirus, or an enterovirus. Additional enveloped virus surface proteins and nonenveloped surface proteins for use in the present disclosure, include, for example, those described in J. M. White, Structures and Mechanisms of Viral Membrane Fusion Proteins, 43 Crit. Rev. Biochem. Mol. Biol. 287 (2009); Boncristiani, H. F., Criado, M. F. and Arruda, E., Respiratory viruses. Encyclopedia of Microbiology, 500 (2009); and M. S. Maginnis, Virus-Receptor Interactions: The Key to Cellular Invasion, 430 J. Mol. Bio. 2590 (2018); each of which is incorporated by reference in its entirety. Exemplary bacterial pathogens include, for example, E. coli., listeria, Mycobacterium tuberculosis, streptococcus, or Treponema palladium. Exemplary fungal pathogens include, for example, a candida fungus, an aspergillius fungus, a cryptococcus fungus, or a histoplasma fungus. Such examples are exemplary and do not limit the scope of the present disclosure.

### Definitions

Unless defined otherwise, all technical and scientific terms used herein have the same meaning as is commonly understood by one of skill in the art to which this present disclosure belongs. All patents and publications referred to herein are incorporated by reference.

As used in the specification and claims, the singular form “a”, “an” and “the” includes plural references unless the context clearly dictates otherwise. For example “a viral protein” as used herein in the specification and claims can denote a single viral protein or a plurality of viral proteins.

As used herein, the term “affinity” refers to the non-random interaction of two molecules. The term “affinity” refers to the strength of interactions and can be expressed quantitatively as a dissociation constant (K_{D}). One or both of the two molecules can be a protein (e.g. a viral protein or a receptor protein). Binding affinity (i.e., K_{D}) can be determined using standard techniques. In some cases, a K_{D} is determined by surface plasmon resonance (SPR) at 37° C. For example, the affinity can be a measure of the strength of the binding of an individual epitope with an antibody molecule. In some embodiments, “diminished affinity” refers to an absence of non-random interaction of two molecules. In some embodiments. “diminished affinity” refers at least 1-order of magnitude lower binding affinity as compared to the binding affinity between two molecules that specifically bind to each other. In some embodiments. “diminished affinity” refers to a substantial absence of specific binding of two molecules (e.g., a modified form of the viral protein described herein and an neutralizing antibody against the viral protein).

As used herein, the term “antibody” (hereinafter used interchangeably with “immunoglobulin”) can refer to members of the globulin protein superfamily which contain antigen binding sites, and which often comprise binding specificities for particular epitopes. A naturally occurring antibody can comprise at least two heavy chains and at least two light chains. These distinct chains can be distinct polypeptides coupled by disulfide bonds, and can further be glycosylated in a manner which can enhance solubility and diminish degradation and glomerular clearance. A heavy chain can comprise a heavy chain variable region and a heavy chain constant region, while a light chain is generally comprised of a light chain variable region and a light chain constant region. In many antibodies, a domain of a heavy chain constant region or multiple domains of a group of heavy chain constant regions can form a fragment crystallizable (Fc) region. An Fc region of an antibody can mediate its interactions with other components of immune systems, such as antibody receptors. Variable regions of the heavy and light chains generally form Fragment antigen-binding (Fab) regions, which often impart antigen-specific binding affinities.

In some cases, the term “neutralizing antibody” (NAb) refers to an antibody or antigen-binding fragments thereof that binds to a region on an extracellular epitope on a pathogen, where binding interferes with pathogen's infection of a host cell (e.g., entry of the pathogen into a host cell). In some embodiments, for example, neutralizing antibodies bind to a receptor binding domain (RBD) of a viral spike protein (e.g., the receptor binding motif (RBM) subunit of the RBD of SARS-CoV-2 Spike protein or the RBM of various variants thereof) so that the binding of neutralizing antibody disrupts or blocks the binding of the RBD to its natural target, thereby preventing infection of the host cell. In some cases, a natural target of the RBD is a human angiotensin converting enzyme type-II (ACE2).

In some cases, the term “non-neutralizing antibody” (NAb) refers to an antibody or antigen-binding fragments thereof that binds to a specific region on a extracellular epitope on a pathogen, where binding does not interfere with pathogen infection of a host cell (e.g., entry of the pathogen into a host cell). In some cases, the term “non-neutralizing antibody” refers to an antibody or antigen-binding fragments thereof that binds to a portion of a viral spike protein (e.g., RBD of SARS-CoV-2 Spike protein or the RBD of various variants thereof) that does not affect the binding of RBD to its natural target, such as a ACE2, so that viral attachment and pathogenesis can occur.

As used herein, the term “pathogen” denotes an organism, virus, viroid, or prion which can cause or contribute to a disease in a host organism. Often, a pathogen is capable of proliferating within the host organism. The pathogen can directly infect a host organism, for example by colonizing interstitium within the host organism, or can generate agents which cause or contribute to a disease.

The term “protein” includes single-chain polypeptide molecules as well as multiple-polypeptide complexes where individual constituent polypeptides are linked by covalent or non-covalent means. As used herein the term “protein” encompasses both full-length proteins (e.g., those found in nature or having the same length or the same number of amino acids as those found in nature), as well as fragments thereof.

The terms “wild-type” or “WT” as used herein refer to an amino acid sequence or a nucleotide sequence that is found in nature, including allelic variations. A wild-type protein has an amino acid sequence or a nucleotide sequence that has not been intentionally modified.

The term “polypeptide” includes peptides of two or more amino acid residues in length, typically having more than 5, 10 or 20 amino acids.

The terms “substitution” or “mutation” refers to a change to the polypeptide backbone wherein an amino acid occurring naturally in the wild-type sequence of a polypeptide is substituted to another amino acid not naturally occurring at the same position in the said polypeptide. In some cases, a mutation or mutations is introduced to modify a polypeptide's affinity to its receptor thereby altering its activity such that it becomes different from the affinity and activity of the wild-type cognate polypeptide. Mutations can also improve a polypeptide's biophysical properties. Amino acid mutations can be generated using genetic or chemical methods well known in the art. Genetic methods can include site-directed mutagenesis, PCR, gene synthesis and the like. It is contemplated that methods of altering the side chain group of an amino acid by methods other than genetic engineering, such as chemical modification, can also be useful.

“Epitope” as used herein refers to a determinant capable of specific binding to the variable region of an antibody molecule known as a paratope. Epitopes are groupings of molecules such as amino acids or sugar side chains and usually have specific structural characteristics, as well as specific charge characteristics. A single antigen can have more than one epitope. The epitope can comprise amino acid residues directly involved in the binding and other amino acid residues, which are not directly involved in the binding, such as amino acid residues which are effectively blocked by the antigen binding peptide (in other words, the amino acid residue is within the footprint of the antigen binding peptide). Epitopes can be either conformational or linear. An epitope typically includes at least 3, and more usually, at least 5 or 8-10 amino acids. Antibodies that recognize the same epitope can be verified in a simple immunoassay showing the ability of one antibody to block the binding of another antibody to a target antigen, for example “binning”.

As used herein, the term “set” refers to one or more items having a common feature or functionality. For example, as used herein a “set of antibodies” (e.g., “first set of antibodies”) refers to one or more antibodies having a common feature (e.g., being either neutralizing or non-neutralizing with respect to a particular antigen (e.g., viral protein)). Thus, a “first set of antibodies” can include one antibody or a plurality of antibodies, so long as the plurality of antibodies share a common feature of, for example, being non-neutralizing with respect to a given viral protein, and a “second set of antibodies” can include one antibody or a plurality of antibodies that share a common feature of, for example, being neutralizing with respect to the same viral protein. In some embodiments, the first and second sets of antibodies are distinguishable, for example, based on their respective identities as non-neutralizing and neutralizing, respectively, with respect to a particular antigen (e.g., viral protein).

The term “subject” is intended to include living organisms in which an immune response can be elicited (e.g., mammals). A “subject” or “patient,” as used therein, can be a human or non-human mammal. Non-human mammals include, for example, livestock and pets, such as ovine, bovine, porcine, canine, feline and murine mammals. In some cases, a subject is a human.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

“Sequence identity” or “homology” as used herein refers to the percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2. ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or can be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “about,” “nearly” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The phrase “obtaining a biological sample” or “obtaining a sample” as used herein encompasses receipt of the sample from any number of sources, including obtaining a sample from storage. As used herein, this term includes, but is not limited to the initial act of taking the sample from the subject.

### Compositions, Devices, and Methods of Detecting Neutralizing Antibodies

As noted above, the systems, methods and compositions of the present disclosure are generalizable to a number of pathogens, including bacterial, viral, and fungal pathogens. For ease of explanation, the following embodiments are directed at detecting neutralizing antibodies for SARS-CoV-2. However, these embodiments are merely exemplary of the present disclosure and do not limit the general applicability of the methods disclosed herein. Further, references to viral protein below are merely exemplary and are not meant to limit the present disclosure. Thus, features discussed below with respect to the detection of neutralizing antibodies against a viral protein (e.g., unnatural proteins comprising such proteins, fusion proteins, protein stapling, etc.) are generalizable to other pathogen proteins such as bacterial surface proteins, fungal surface proteins, etc.

In some systems for detecting neutralizing antibodies for SARS-CoV-2, the receptor binding domain of the SARS-CoV-2 spike protein (“S-RBD”) is used to detect both neutralizing antibodies and non-neutralizing antibodies, and a co-spotted combination of S-RBD and ACE2, its cognate receptor in humans, is used to detect only non-neutralizing antibodies. As discussed above, the presence or absence of neutralizing antibodies in the sample is determined by subtraction of two signals (i.e., signal from S-RBD/ACE2 co-spot is subtracted from the signal from the S-RBD spot).

In some systems, the S-RBD and S-RBD/ACE2 proteins are directionally oriented on neutravidin pretreated nitrocellulose using S-RBD having a single biotin remote from the S-RBD/ACE2 interaction site. While the association of S-RBD and ACE2 has a moderately high interaction strength (K_{D}≈20-40 nanomolar), there remains the possibility of dissociation between S-RBD and ACE2 during the process of spotting, drying, and probing with plasma. As discussed above, a microarray spotted with both S-RBD and S-RBD/ACE2 complex, probed with a neutralizing monoclonal IgG antibody, could, for example, demonstrate a decrease in antibody binding signal for the S-RBD/ACE2 complex compared to the S-RBD. If a significant proportion of the S-RBD/ACE2 complex dissociates, the neutralizing antibodies can bind the S-RBD, even in the areas co-spotted with ACE2. The resulting background noise in the data significantly decreases the use of such assays for the detection of neutralizing antibodies.

Although S-RBD and ACE2 are each produced as individual polypeptides, they complex together at relatively high concentrations (e.g., one micromolar concentration of each to maintain virtually 100% of S-RBD and ACE2 complexed together). The interaction between S-RBD and ACE2 can be disrupted upon dilution, leading to dissociation of the complex. The estimated half-life of the complex is ˜ 10-20 minutes if proteins are diluted. Significant dilution occurs throughout the process of detecting neutralizing antibodies, including microarray incubation in plasma, secondary antibody addition, and washes between each step. Thus, during methods of neutralizing antibody detection, there is significant risk of dissociation of the S-RBD-ACE2 complex, leading to diminish sensitivity for neutralizing antibodies.

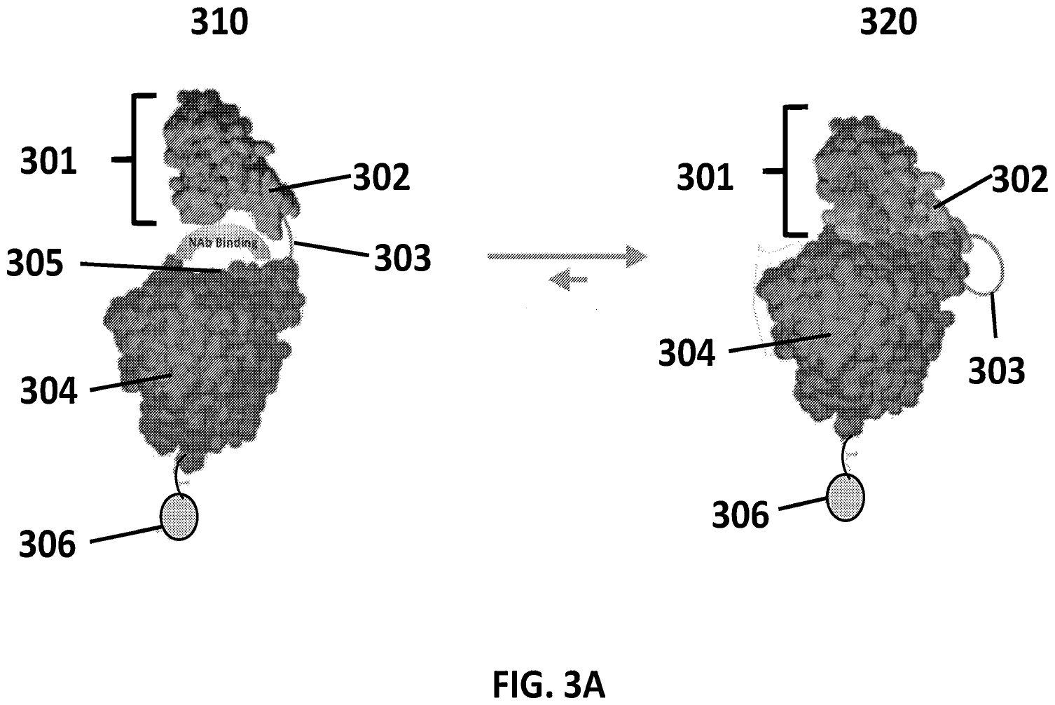

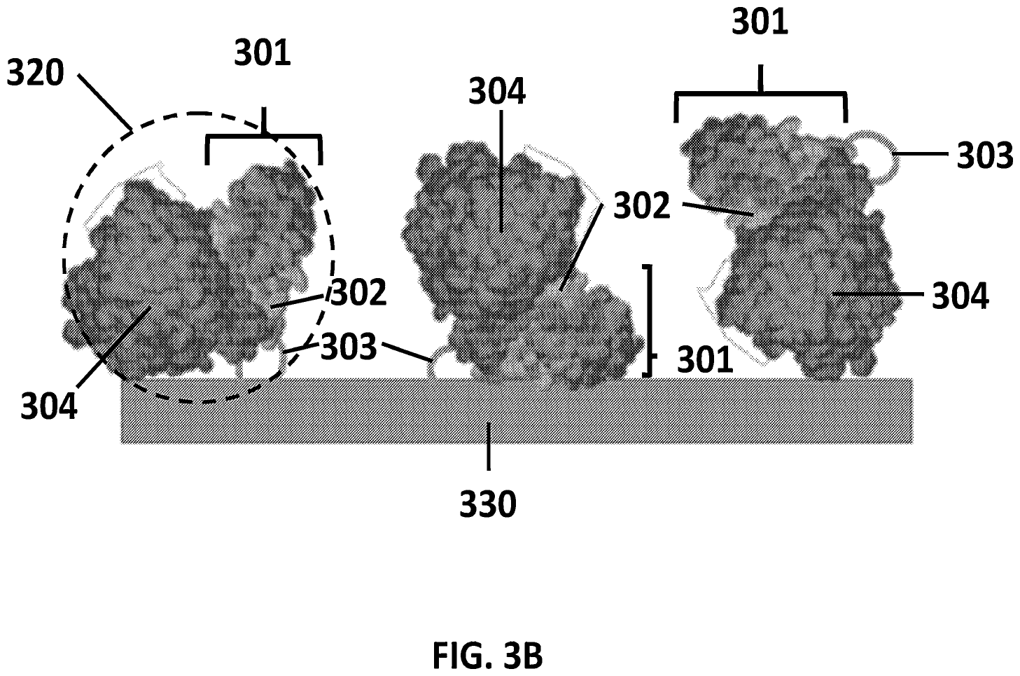

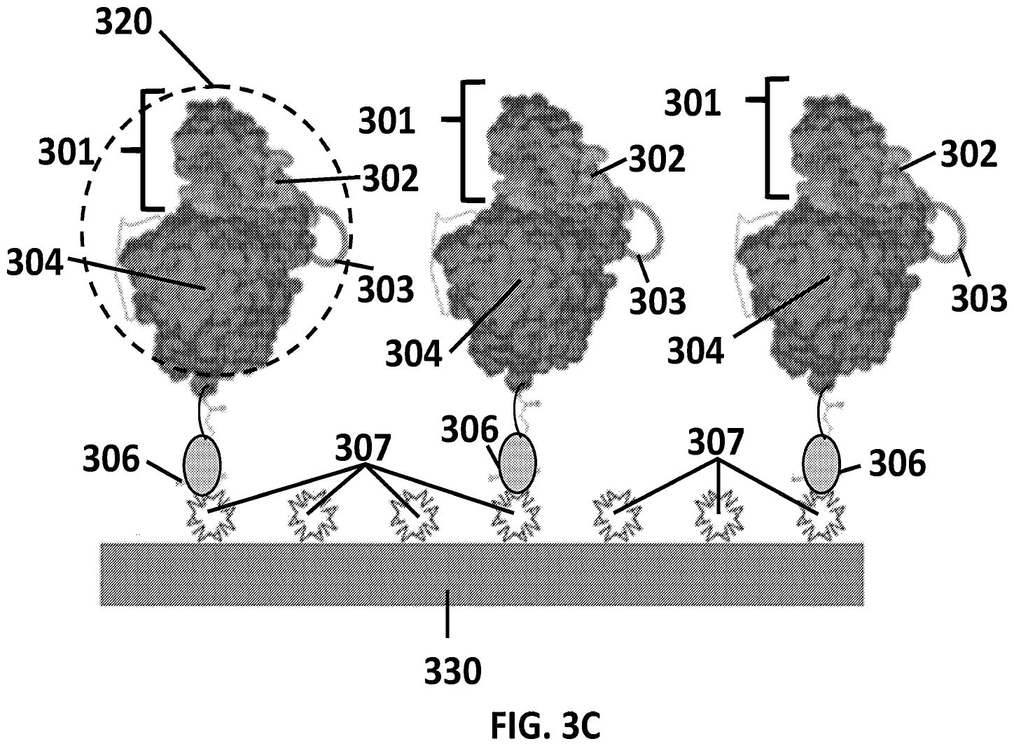

In embodiments of the present disclosure for use in the detection of SARS-CoV-2 neutralizing antibodies, a protein complex comprising S-RBD covalently linked to ACE2 in order to negate the relatively shortened the half-life of the S-RBD and ACE2 complex. In some embodiments, the covalent S-RBD/ACE2 complex utilize the polypeptides (S-RBD and ACE2) have been genetically fused by means of a short linker to create a single polypeptide (fusion protein) (FIGS. 3A-3C). In some embodiments, the covalent protein complex comprises a covalent bond between a first amino acid residue on the S-RBD and a second amino acid residue on the ACE2 protein (stapled protein complex). In various embodiments, one or both of the fusion protein and stapled protein strategies can be utilized. For example, a fusion protein comprising S-RBD, ACE2, and a linker could be used with or without a covalent “staple” between the S-RBD and ACE2. Similarly, in various embodiments, a stapled protein complex between S-RBD and ACE2 can be a single polypeptide (i.e., also a fusion protein), or can be two separate polypeptides. Each of these approaches is described in detail below.

Table 1, below, provides a brief description of the sequences referenced in the present disclosure, as well as the location of key domains referred to herein.

TABLE 1

| SEQ | | | ACE2 | ACE2 | |
|---|---|---|---|---|---|
| ID | Description of | S-RBD | Soluble | Collectrin | Linker |
| NO: | Protein Sequence | Domain | Domain | Domain | Domain |
| 1 | Wild-type SARS-COV2 | aa 319- | — | — | — |
| | Spike Protein | 541 | | | |
| 2 | Wild-type Canonical | — | aa 18- | aa 615- | — |
| | Isoform of hACE2 | | 614 | 740 | |
| 3 | Fusion Protein 3A | aa 41- | aa 298- | aa 894- | aa 264- |
| | (Expressed in | 263 | 893 | 1020 | 297 |
| | Baculovirus System) | | | | |
| 4 | M3D2 Fusion Protein | aa 20- | aa 284- | — | aa 243- |
| | (Expressed in | 242 | 880 | | 283 |
| | Mammalian System) | | | | |
| 5 | M3D4 Stapled Fusion | aa 20- | aa 284- | — | aa 243- |
| | Protein (Expressed in | 242 | 880 | | 283 |
| | Mammalian System) | | | | |

As shown in Table 1, SEQ ID NO: 1 provides the amino acid for the SARS-COV2 Spike protein from the UniProt database. The RBD of the spike protein corresponds to amino acids 319 to 541 of SEQ ID NO:1.

The canonical isoform of human ACE2 protein sequence from the UniProt database is provided as SEQ ID NO:2. The soluble domain of ACE2 protein sequence corresponds to amino acids 18 to 614 of SEQ ID NO:2. The collectrin domain of ACE2 protein sequence corresponds to amino acids 615 to 740 of SEQ ID NO:2.

The amino acid sequence for one protein of the present disclosure, referred to herein as fusion protein 3A (expressed in a Baculovirus system), is provided as SEQ ID NO: 3. The fusion protein 3A comprises a SARS-COV2 Spike RBD domain (amino acids 41 to 263 of SEQ ID NO:3), a polypeptide linker (amino acids 264 to 297 of SEQ ID NO:3), an ACE2 soluble domain (amino acids 298 to 893 of SEQ ID NO:3), and an ACE2 collectrin domain (amino acids 894 to 1020 of SEQ ID NO:3).

The amino acid sequence for another protein of the present disclosure, referred to herein as fusion protein M3D2 (expressed in a Mammalian system), is provided as SEQ ID NO: 4. The fusion protein M3D2 comprises a SARS-COV2 Spike RBD domain (amino acids 20 to 242 of SEQ ID NO:4), a polypeptide linker (amino acids 243 to 283 of SEQ ID NO:4), and an ACE2 soluble domain (amino acids 284 to 880 of SEQ ID NO:4).

The amino acid sequence for another protein of the present disclosure, referred to herein as fusion protein M3D4 (expressed in a Mammalian system), is provided as SEQ ID NO: 5. The fusion protein M3D4 comprises a SARS-COV2 Spike RBD domain (amino acids 20 to 242 of SEQ ID NO:5), a polypeptide linker (amino acids 243 to 283 of SEQ ID NO:5), and an ACE2 soluble domain (amino acids 284 to 880 of SEQ ID NO:5). In some embodiments, the SEQ ID NO:5 comprise a SARS-COV2 Spike RBD domain (amino acids 20 to 242 of SEQ ID NO:5 with a 196G to C mutation. In some embodiments, the SEQ ID NO: 5 comprise an ACE2 soluble domain (amino acids 284 to 880 of SEQ ID NO:5 with a 304D to C mutation. The fusion protein M3D4 forms an intrachain disulfide bond between two cysteines: 196C and 304C. In some cases, the fusion protein M3D4 forms an intrachain disulfide bond between two cysteines: 196C and 304C. This disulfide bond covalently couples the interface of the ACE2 soluble domain to the S-RBD, thereby resulting in a “stapled” fusion protein.

### Unnatural Protein Comprising Linkers

In some embodiments, the devices and methods disclosed herein utilize an unnatural protein comprising (i) a pathogen protein, (ii) a receptor protein having an affinity for the pathogen protein; and (iii) a linker connecting the pathogen protein to the receptor protein. In some embodiments, the devices and methods disclosed herein utilize a fusion protein comprising (i) a pathogen protein, (ii) a receptor protein having an affinity for the pathogen protein; and (iii) a linker connecting the pathogen protein to the receptor protein.

In some embodiments, the devices and methods disclosed herein utilize an unnatural protein comprising (i) a viral protein, (ii) a receptor protein having an affinity for the viral protein; and (iii) a linker connecting the viral protein to the receptor protein. In some embodiments, the devices and methods disclosed herein utilize a fusion protein comprising (i) a viral protein, (ii) a receptor protein having an affinity for the viral protein; and (iii) a linker connecting the viral protein to the receptor protein.

In some embodiments, the unnatural protein selectively binds to non-neutralizing antibodies of the viral protein over neutralizing antibodies of the viral protein. In some embodiments, the unnatural protein comprises at least 1-order of magnitude greater binding affinity for the non-neutralizing antibodies than for the neutralizing antibodies. In some embodiments, the unnatural protein comprises, for example, at least 1-order of magnitude, at least 2-orders of magnitude, at least 3 orders of magnitude, or at least 5-orders of magnitude greater binding affinity for the non-neutralizing antibodies than for the neutralizing antibodies. In some embodiments, the unnatural protein comprises between 1-order of magnitude and 4-orders of magnitude greater binding affinity for the non-neutralizing antibodies than for the neutralizing antibodies. In some embodiments, the unnatural protein comprises, for example, between 1-order of magnitude and 4-orders of magnitude, between 1-order of magnitude and 3-orders of magnitude, between 1-order of magnitude and 2-orders of magnitude, between 2-order of magnitude and 4-orders of magnitude, between 2-order of magnitude and 3-orders of magnitude greater binding affinity for the non-neutralizing antibodies than for the neutralizing antibodies.

In some embodiments, the neutralizing antibodies comprise at least 1-order of magnitude lower binding affinity for the unnatural protein than for the viral protein in an absence of the receptor protein and the linker. In some embodiments, the neutralizing antibodies comprise for example, at least 1-order of magnitude, at least 2-orders of magnitude, at least 3 orders of magnitude, or at least 5-orders of magnitude lower binding affinity for the unnatural protein than for the viral protein in an absence of the receptor protein and the linker. In some embodiments, the neutralizing antibodies comprise between 1-order of magnitude and 4-orders of magnitude lower binding affinity for the unnatural protein than for the viral protein in an absence of the receptor protein and the linker. In some embodiments, the neutralizing antibodies comprise for example, between 1-order of magnitude and 4-orders of magnitude, between 1-order of magnitude and 3-orders of magnitude, between 1-order of magnitude and 2-orders of magnitude, between 2-order of magnitude and 4-orders of magnitude, between 2-order of magnitude and 3-orders of magnitude lower binding affinity for the unnatural protein than for the viral protein in an absence of the receptor protein and the linker.

In some embodiments, the neutralizing antibodies possess affinities for a receptor binding domain of the viral protein. In some embodiments, the neutralizing antibodies possess affinities for a receptor binding motif of the viral protein.

In some embodiments, the affinity of the receptor protein is for at least a portion of the receptor protein binding domain of the viral protein. In some embodiments, the affinity of the receptor protein for the viral protein is at least 1 μM. In this case, greater binding affinity results in a lower concentration required for the same degree of binding (e.g., 1 nM binding affinity). In some embodiments, the affinity of the receptor protein for the viral protein is greater than 1 μM. In some embodiments, the affinity of the receptor protein for the viral protein is greater than, e.g. 50, 40, 30, 20, 10, 9, 8, 7, 6, 5, 4, 3, 2, or, 1 μM. In some embodiments, the affinity of the receptor protein for the viral protein is greater than, e.g. 900, 800, 700, 600, 500, 400, 300, 200, or, 100 nM. In some embodiments, the affinity of the receptor protein for the viral protein is greater than, e.g. 90, 80, 70, 60, 50, 40, 30, 20, or, 10 nM. In some embodiments, the affinity of the receptor protein for the viral protein is between 10 nM and 1 μM. In some embodiments, the affinity of the receptor protein for the viral protein is between 100 nM and 10 μM. In some embodiments, the affinity of the receptor protein for the viral protein is between 100 nM and 50 μM.

In some embodiments, the affinity the receptor protein for the viral protein equals to a dissociation constant (K_{D}) of less than 1 μM, 800 nM, 600 nM, 400 nM, 200 nM, 150 nM, 100 nM, 80 nM, 60 nM, 50 nM, 40 nM, or 20 nM. In this case, greater binding affinity results in a lower concentration required for the same degree of binding (e.g., a lower K_{D}). In some embodiments, the affinity the receptor protein for the viral protein equals to a dissociation constant (K_{D}) of less than 1 μM. In some embodiments, the affinity of the receptor protein for the viral protein equals to a dissociation constant (K_{D}) of less than, e.g., 900, 800, 700, 600, 500, 400, 300, 200, or, 100 nM. In some embodiments, the affinity of the receptor protein for the viral protein equals to a dissociation constant (K_{D}), e.g. 90, 80, 70, 60, 50, 40, 30, 20, or, 10 nM. In some embodiments, the affinity of the receptor protein for the viral protein equals to a dissociation constant (K_{D}) between 10 nM and 1 μM. In some embodiments, the affinity of the receptor protein for the viral protein equals to a dissociation constant (K_{D}) between 100 nM and 10 μM. In some embodiments, the affinity of the receptor protein for the viral protein equals to a dissociation constant (K_{D}) between 100 nM and 50 μM.

In some embodiments, the viral protein comprises a viral surface protein. In some embodiments, the viral protein comprises a viral coat protein. In some embodiments, the viral protein is an enveloped virus surface protein. In some embodiments, the viral protein is a nonenveloped virus surface protein. In some embodiments, the viral protein is a fragment of a full-length viral protein. In some embodiments, the viral protein is a full-length viral protein. In some embodiments, the fragment of the full-length viral protein comprises a complete domain of a viral protein. In some embodiments, the fragment of the full-length viral protein comprises a portion of a domain of a viral protein. In some embodiments, the fragment of the full-length viral protein is a functional fragment. For example, in some embodiments, the fragment of the full-length viral protein comprises a complete receptor binding motif of the viral protein that retains its ability to bind to the corresponding host cell receptor. In some embodiments, the viral protein comprises at least a portion of a receptor binding domain of the viral protein.

In some embodiments, the viral protein comprises at least a portion of a viral protein derived from a virus selected from a coronavirus, an influenza virus, a zikavirus, a polyomoavirus, and an HIV virus. In some embodiments, the viral protein comprises at least a portion of a viral protein derived from a respiratory virus. In some embodiments, the viral protein comprises at least a portion of a viral protein derived from a virus selected from a coronavirus and an influenza virus. In some embodiments, the viral protein comprises at least a portion of a viral protein derived from a coronavirus. In some embodiments, the viral protein comprises at least a portion of a viral protein derived from SARS-CoV-2.

In some embodiments, the viral protein comprises at least a portion of a protein selected from the group consisting of an influenza A hemagglutinin protein, an influenza A neuraminidase protein, a MERS-CoV spike protein, a SARS-CoV2 spike protein, a zikavirus E protein, a polyomavirus VP1 protein; an HIV Env protein. In some embodiments, the viral protein comprises at least a portion of a protein selected from the group consisting of an influenza A hemagglutinin protein, an influenza A neuraminidase protein, a MERS-CoV spike protein, and a SARS-CoV2 spike protein. In some embodiments, the viral protein comprises at least a portion of a protein selected from the group consisting of an influenza A hemagglutinin protein, a MERS-CoV spike protein, and a SARS-CoV2 spike protein. In some embodiments, the viral protein comprises at least a portion of a protein selected from the group consisting of a MERS-CoV spike protein, and a SARS-CoV2 spike protein. In some embodiments, the viral protein comprises at least a portion of a SARS-CoV2 spike protein.

In some embodiments, the viral protein is a coronavirus viral surface protein. In some embodiments, the viral protein is an influenza viral surface protein. In some embodiments, the coronavirus viral surface protein is a SARS-CoV2 surface protein. In some embodiments, the coronavirus viral surface protein is a MERS-CoV surface protein.

In some embodiments, the viral protein comprises amino acids nos. 319 to 541 of SEQ ID NO: 1. In some embodiments, the viral protein comprises a sequence having, e.g., at least 70%, at least 75%, at least 80%, at least 85%, at least 90%, at least 95%, at least 97%, at least 98%, at least 99% sequence identity (or homology) to amino acids nos. 319 to 541 of SEQ ID NO:1. In some embodiments, the viral protein comprises a sequence having at least 70% sequence identity (or homology) to amino acids nos. 319 to 541 of SEQ ID NO: 1. In some embodiments, the viral protein comprises a sequence having at least 80% sequence identity (or homology) to amino acids nos. 319 to 541 of SEQ ID NO:1. In some embodiments, the viral protein comprises a sequence having at least 90% sequence identity (or homology) to amino acids nos. 319 to 541 of SEQ ID NO: 1. In some embodiments, the viral protein comprises a sequence having at least 95% sequence identity (or homology) to amino acids nos. 319 to 541 of SEQ ID NO: 1. In some embodiments, the viral protein comprises a sequence having at least 97% sequence identity (or homology) to amino acids nos. 319 to 541 of SEQ ID NO: 1. In some embodiments, the viral protein comprises a sequence having at least 98% sequence identity (or homology) to amino acids nos. 319 to 541 of SEQ ID NO: 1. In some embodiments, the viral protein comprises a sequence having at least 99% sequence identity (or homology) to amino acids nos. 319 to 541 of SEQ ID NO:1.

In some embodiments, the viral protein comprises amino acids nos. 20 to 242 of SEQ ID NO:4. In some embodiments, the viral protein comprises a sequence having, e.g., at least 70%, at least 75%, at least 80%, at least 85%, at least 90%, at least 95%, at least 97%, at least 98%, at least 99% sequence identity (or homology) to amino acids nos. 20 to 242 of SEQ ID NO:4. In some embodiments, the viral protein comprises a sequence having at least 70% sequence identity (or homology) to amino acids nos. 20 to 242 of SEQ ID NO:4. In some embodiments, the viral protein comprises a sequence having at least 80% sequence identity (or homology) to amino acids nos. 20 to 242 of SEQ ID NO:4. In some embodiments, the viral protein comprises a sequence having at least 90% sequence identity (or homology) to amino acids nos. 20 to 242 of SEQ ID NO:4. In some embodiments, the viral protein comprises a sequence having at least 95% sequence identity (or homology) to amino acids nos. 20 to 242 of SEQ ID NO:4. In some embodiments, the viral protein comprises a sequence having at least 97% sequence identity (or homology) to amino acids nos. 20 to 242 of SEQ ID NO:4. In some embodiments, the viral protein comprises a sequence having at least 98% sequence identity (or homology) to amino acids nos. 20 to 242 of SEQ ID NO:4. In some embodiments, the viral protein comprises a sequence having at least 99% sequence identity (or homology) to amino acids nos. 20 to 242 of SEQ ID NO:4.

In some embodiments, the receptor protein is a fragment of a full-length receptor protein. In some embodiments, the receptor protein is a full-length receptor protein. In some embodiments, the fragment of the full-length receptor protein comprises a complete domain of a receptor protein. In some embodiments, the fragment of the full-length receptor protein comprises a portion of a domain of a receptor protein. In some embodiments, the fragment of the full-length receptor protein is a functional fragment. For example, in some embodiments, the fragments of the full-length receptor protein comprise a complete antigen binding motif of the receptor protein that retains its ability to bind to the cognate antigen on the surface of the pathogen. In some embodiments, the receptor protein comprises at least a portion of an antigen binding domain of the receptor protein.

In some embodiments, the receptor protein comprises at least a portion of a natural binding target for the viral surface protein. In some embodiments, the receptor protein comprises at least a portion of a human protein. In some embodiments, the receptor protein comprises at least a portion of a human cell-surface glycoprotein. In some embodiments, the receptor protein comprises at least a portion of a human cell-surface glycoprotein comprising sialic acid. In some embodiments, the receptor protein comprises at least a portion of an angiotensin converting enzyme 2 (ACE2). In some embodiments, the receptor protein comprises a soluble fragment of ACE2.

In some embodiments, the receptor protein comprises amino acids nos. 18 to 614 of SEQ ID NO:2. In some embodiments, the receptor protein comprises a sequence having, e.g., at least 70%, at least 75%, at least 80%, at least 85%, at least 90%, at least 95%, 18 to 614 of SEQ ID NO:2. In some embodiments, the receptor protein comprises a sequence having at least 70% sequence identity (or homology) to amino acids nos. 18 to 614 of SEQ ID NO: 2. In some embodiments, the receptor protein comprises a sequence having at least 80% sequence identity (or homology) to amino acids nos. 18 to 614 of SEQ ID NO:2. In some embodiments, the receptor protein comprises a sequence having at least 90% sequence identity (or homology) to amino acids nos. 18 to 614 of SEQ ID NO:2. In some embodiments, the receptor protein comprises a sequence having at least 95% sequence identity (or homology) to amino acids nos. 18 to 614 of SEQ ID NO:2. In some embodiments, the receptor protein comprises a sequence having at least 97% sequence identity (or homology) to amino acids nos. 18 to 614 of SEQ ID NO:2. In some embodiments, the receptor protein comprises a sequence having at least 98% sequence identity (or homology) to amino acids nos. 18 to 614 of SEQ ID NO:2. In some embodiments, the receptor protein comprises a sequence having at least 99% sequence identity (or homology) to amino acids nos. 18 to 614 of SEQ ID NO:2.

In some embodiments, the receptor protein comprises amino acids nos. 284 to 870 of SEQ ID NO:4. In some embodiments, the receptor protein comprises a sequence having. e.g., at least 70%, at least 75%, at least 80%, at least 85%, at least 90%, at least 95%, at least 97%, at least 98%, at least 99% sequence identity (or homology) to amino acids nos. 284 to 870 of SEQ ID NO:4. In some embodiments, the receptor protein comprises a sequence having at least 70% sequence identity (or homology) to amino acids nos. 284 to 870 of SEQ ID NO:4. In some embodiments, the receptor protein comprises a sequence having at least 80% sequence identity (or homology) to amino acids nos. 284 to 870 of SEQ ID NO:4. In some embodiments, the receptor protein comprises a sequence having at least 90% sequence identity (or homology) to amino acids nos. 284 to 870 of SEQ ID NO:4. In some embodiments, the receptor protein comprises a sequence having at least 95% sequence identity (or homology) to amino acids nos. 284 to 870 of SEQ ID NO:4. In some embodiments, the receptor protein comprises a sequence having at least 97% sequence identity (or homology) to amino acids nos. 284 to 870 of SEQ ID NO:4. In some embodiments, the receptor protein comprises a sequence having at least 98% sequence identity (or homology) to amino acids nos. 284 to 870 of SEQ ID NO:4. In some embodiments, the receptor protein comprises a sequence having at least 99% sequence identity (or homology) to amino acids nos. 284 to 870 of SEQ ID NO:4. In some embodiments, the receptor protein comprises a sequence having 100% sequence identity (or homology) to amino acids nos. 284 to 870 of SEQ ID NO:4.

In some embodiments, the receptor protein comprises amino acids nos. 284 to 880 of SEQ ID NO:4. In some embodiments, the receptor protein comprises a sequence having, e.g., at least 70%, at least 75%, at least 80%, at least 85%, at least 90%, at least 95%, at least 97%, at least 98%, at least 99% sequence identity (or homology) to amino acids nos. 284 to 880 of SEQ ID NO:4. In some embodiments, the receptor protein comprises a sequence having at least 70% sequence identity (or homology) to amino acids nos. 284 to 880 of SEQ ID NO:4. In some embodiments, the receptor protein comprises a sequence having at least 80% sequence identity (or homology) to amino acids nos. 284 to 880 of SEQ ID NO:4. In some embodiments, the receptor protein comprises a sequence having at least 90% sequence identity (or homology) to amino acids nos. 284 to 880 of SEQ ID NO:4. In some embodiments, the receptor protein comprises a sequence having at least 95% sequence identity (or homology) to amino acids nos. 284 to 880 of SEQ ID NO:4. In some embodiments, the receptor protein comprises a sequence having at least 97% sequence identity (or homology) to amino acids nos. 284 to 880 of SEQ ID NO:4. In some embodiments, the receptor protein comprises a sequence having at least 98% sequence identity (or homology) to amino acids nos. 284 to 880 of SEQ ID NO:4. In some embodiments, the receptor protein comprises a sequence having at least 99% sequence identity (or homology) to amino acids nos. 284 to 880 of SEQ ID NO:4.

In some embodiments, the linker connects the N-terminus of the receptor protein to the C-terminus of the viral protein. In some embodiments, the linker comprises a polypeptide linker (e.g., a fusion protein). In some embodiments, the linker comprises a water-soluble polymer and/or oligomer. In some embodiments, the linker comprises polyethylene glycol. In some embodiments, the linker comprises a polypeptide linker. In some embodiments, the linker comprises a flexible polypeptide linker. In some embodiments, the linker comprises a -GGGGS- pentapeptide motif (SEQ ID NO: 6). In some embodiments, the linker comprises at least two, at least three, or at least four-GGGGS-pentapeptide motifs (SEQ ID NO: 6).

In some embodiments, the linker comprises an endopeptidase recognition sequence. Many endopeptidase recognition sequences are known in the art and are compatible with the embodiments of the present disclosure. For example, in some embodiments, the linker comprises a TEV protease recognition sequence.

In some embodiments, the linker comprises amino acids nos. 243 to 283 of SEQ ID NO:4. In some embodiments, the linker comprises a sequence having, e.g., at least 70%, at least 75%, at least 80%, at least 85%, at least 90%, at least 95%, at least 97%, at least 98%, at least 99% sequence identity (or homology) to amino acids nos. 243 to 283 of SEQ ID NO: 4. In some embodiments, the linker comprises a sequence having at least 70% sequence identity (or homology) to amino acids nos. 243 to 283 of SEQ ID NO:4. In some embodiments, the linker comprises a sequence having at least 80% sequence identity (or homology) to amino acids nos. 243 to 283 of SEQ ID NO:4. In some embodiments, the linker comprises a sequence having at least 90% sequence identity (or homology) to amino acids nos. 243 to 283 of SEQ ID NO:4. In some embodiments, the linker comprises a sequence having at least 95% sequence identity (or homology) to amino acids nos. 243 to 283 of SEQ ID NO: 4. In some embodiments, the linker comprises a sequence having at least 97% sequence identity (or homology) to amino acids nos. 243 to 283 of SEQ ID NO:4. In some embodiments, linker comprises a sequence having at least 98% sequence identity (or homology) to amino acids nos. 243 to 283 of SEQ ID NO:4. In some embodiments, the linker comprises a sequence having at least 99% sequence identity (or homology) to amino acids nos. 243 to 283 of SEQ ID NO:4.

In some embodiments, the viral protein, the receptor protein, and the linker are a single expression construct. In some embodiments, the viral protein of the unnatural protein comprises a substantially similar conformation as the viral protein in an absence of the receptor protein and the linker. In such embodiments, the viral protein portion of the unnatural protein will bind to the receptor protein with a similar affinity to, for example, the native viral receptor.

FIG. 3A provides an example of a species for detecting non-neutralizing antibodies of COVID-19 S-protein. The complex comprises a COVID-19 S-protein receptor binding domain (S-RBD) 301, including the receptor binding motif (RBM) subunit 302 configured for (angiotensin converting enzyme 2) ACE2 binding. The COVID-19 S-protein receptor binding domain 301 is coupled to a fragment of an ACE2 protein (e.g., the soluble domain of an ACE2 protein) 303 by a linker 303. In some embodiments, the linker is a peptide linker which is expressed coupled to the COVID-19 S-protein S-RBD 301 and the fragment of the ACE2 protein 303. The length and attachment positions of the linker 303 (e.g., the linker can be coupled to the C-terminus of the COVID-19 S-protein RBD 301 and the N-terminus of ACE2 304) can allow the S-protein RBM 302 to bind its native target on ACE2. Binding of the N-terminus of ACE2 304 to S-RBD 301 effectively blocks neutralizing antibody region 305, thereby preventing neutralizing antibodies from binding to modified protein 310. Accordingly, as depicted by structure 320, the species can comprise the S-protein bound to ACE2, such that an S-protein neutralizing antibody epitope (e.g., the RBM or a portion thereof) is inaccessible for antibody binding. In some embodiments, the complex can comprises a functional handle 306, such as a C-terminal biotin, which facilitates its capture (e.g., by streptavidin). Structure 310 provides an alternate conformation of the species, in which the S-protein RBD 301 is not bound to ACE2 304.

In some embodiments, the unnatural protein comprises SEQ ID NO:3. In some embodiments, the unnatural protein comprises a sequence having. e.g., at least 70%, at least 75%, at least 80%, at least 85%, at least 90%, at least 95%, at least 97%, at least 98%, at least 99% sequence identity (or homology) to SEQ ID NO:3. In some embodiments, the unnatural protein consists essentially of SEQ ID NO:3. In some embodiments, the unnatural protein consists essentially of a sequence having, e.g., at least 70%, at least 75%, at least 80%, at least 85%, at least 90%, at least 95%, at least 97%, at least 98%, at least 99% sequence identity (or homology) to SEQ ID NO:3.

In some embodiments, the unnatural protein comprises amino acids nos. 41 to 1020 of SEQ ID NO:3. In some embodiments, the unnatural protein comprises a sequence having, e.g., at least 70%, at least 75%, at least 80%, at least 85%, at least 90%, at least 95%, at least 97%, at least 98%, at least 99% sequence identity (or homology) to amino acids nos. 41 to 1020 of SEQ ID NO:3. In some embodiments, the unnatural protein comprises a sequence having at least 70% sequence identity (or homology) to amino acids nos. 41 to 1020 of SEQ ID NO:3. In some embodiments, the unnatural protein comprises a sequence having at least 80% sequence identity (or homology) to amino acids nos. 41 to 1020 of SEQ ID NO: 3. In some embodiments, the unnatural protein comprises a sequence having at least 90% sequence identity (or homology) to amino acids nos. 41 to 1020 of SEQ ID NO:3. In some embodiments, the unnatural protein comprises a sequence having at least 95% sequence identity (or homology) to amino acids nos. 41 to 1020 of SEQ ID NO:3. In some embodiments, the unnatural protein comprises a sequence having at least 97% sequence identity (or homology) to amino acids nos. 41 to 1020 of SEQ ID NO:3. In some embodiments, unnatural protein comprises a sequence having at least 98% sequence identity (or homology) to amino acids nos. 41 to 1020 of SEQ ID NO:3. In some embodiments, the unnatural protein comprises a sequence having at least 99% sequence identity (or homology) to amino acids nos. 41 to 1020 of SEQ ID NO:3.

In some embodiments, the unnatural protein comprises SEQ ID NO:4. In some embodiments, the unnatural protein comprises a sequence having, e.g., at least 70%, at least 75%, at least 80%, at least 85%, at least 90%, at least 95%, at least 97%, at least 98%, at least 99% sequence identity (or homology) to SEQ ID NO:4. In some embodiments, the unnatural protein consists essentially of SEQ ID NO:4. In some embodiments, the unnatural protein consists essentially of a sequence having, e.g., at least 70%, at least 75%, at least 80%, at least 85%, at least 90%, at least 95%, at least 97%, at least 98%, at least 99% sequence identity (or homology) to SEQ ID NO:4.

In some embodiments, the unnatural protein comprises amino acids nos. 20 to 880 of SEQ ID NO:4. In some embodiments, the unnatural protein comprises a sequence having, e.g., at least 70%, at least 75%, at least 80%, at least 85%, at least 90%, at least 95%, at least 97%, at least 98%, at least 99% sequence identity (or homology) to amino acids nos. 20 to 880 of SEQ ID NO:4. In some embodiments, the unnatural protein comprises a sequence having at least 70% sequence identity (or homology) to amino acids nos. 20 to 880 of SEQ ID NO:4. In some embodiments, the unnatural protein comprises a sequence having at least 80% sequence identity (or homology) to amino acids nos. 20 to 880 of SEQ ID NO:4. In some embodiments, the unnatural protein comprises a sequence having at least 90% sequence identity (or homology) to amino acids nos. 20 to 880 of SEQ ID NO:4. In some embodiments, the unnatural protein comprises a sequence having at least 95% sequence identity (or homology) to amino acids nos. 20 to 880 of SEQ ID NO:4. In some embodiments, the unnatural protein comprises a sequence having at least 97% sequence identity (or homology) to amino acids nos. 20 to 880 of SEQ ID NO:4. In some embodiments, unnatural protein comprises a sequence having at least 98% sequence identity (or homology) to amino acids nos. 20 to 880 of SEQ ID NO:4. In some embodiments, the unnatural protein comprises a sequence having at least 99% sequence identity (or homology) to amino acids nos. 20 to 880 of SEQ ID NO:4.

In some embodiments, the unnatural protein and/or fusion protein is immobilized on a surface. In some embodiments, the surface comprises a nitrocellulose membrane. In some embodiments, the surface comprises a coating. In some embodiments, the coating comprises a biotin-binding agent. In some embodiments, the coating comprises a biotin-binding protein. In some embodiments, the coating comprises an avidin protein. In some embodiments, the surface comprises a neutravidin protein. In some embodiments, the surface comprises a streptavidin protein. In some embodiments, the biotin-binding agent is an avidin protein or a related protein, such as, for example, avidin, streptavidin, neutravidin, ExtrAvidin (Sigma Chemical Co.), or NeutraLite Avidin (Belovo Chemicals). In some embodiments, the biotin-binding agent is a biotin-binding antibody or a portion thereof.

In some embodiments, the unnatural protein is coupled to the surface by a biotin binding motif. In some embodiments, the biotin binding motif is located, for example on any biotin-binding agent disclosed herein. In some embodiments, the unnatural protein is covalently coupled to biotin. Covalent coupling of biotin to the unnatural protein facilitates immobilization of the unnatural protein on the surface via the interaction between biotin and the biotin binding motif.

FIG. 3B provides an example of a microarray comprising species 320 of FIG. 3A coupled directly to a surface 330, such as nitrocellulose. In some embodiments, such an array is generated by depositing the species 320 on the surface, which results in the species 320 adopting random orientations.

FIG. 3C provides an example of a microarray comprising species 320 of FIG. 3A coupled indirectly to a surface 330 through its functional handle 306. In some embodiments, the surface 330 comprises a binding agent 307, such as, e.g., streptavidin or neutravidin, configured to bind the functional handle 306. In some embodiments, the functional handle 306 spaces the species 320 from the surface 330. The species 320 can comprise greater conformational and orientational flexibility relative to the format outlined in FIG. 3B.

In some embodiments, the unnatural protein is randomly oriented on the surface. In some embodiments, the unnatural protein is distributed on the surface in discrete regions (e.g., spots). In some embodiments, the surface comprises a plurality of discrete regions for binding the unnatural protein. In some embodiments, the plurality of discrete regions is arranged in a grid (e.g., a rectilinear grid, a simple grid, or a hexagonal grid). In some embodiments, the unnatural protein is randomly oriented within the discrete region for unnatural protein binding.

In some embodiments, the unnatural protein is substantially inert to human blood proteases. In some embodiments, the unnatural protein will not substantially cleave in the presence of a sample derived from human blood.

### Protein Stapling

In some embodiments, the devices and methods disclosed herein utilize an unnatural protein comprising a pathogen protein and a receptor protein having a binding affinity for the pathogen protein, wherein the pathogen protein is covalently linked to the receptor protein via a covalent bond between a first amino acid residue on the pathogen protein, and a second amino acid residue on the receptor protein. In some embodiments, the first amino acid residue and the second residue are each located at a binding interface between the viral protein and the receptor protein. In some embodiments, at least one of the first amino acid residue and the second amino acid residue is not present in a wild-type sequence of the pathogen protein or the receptor protein.

In some embodiments, the devices and methods disclosed herein utilize an unnatural protein comprising a viral protein and a receptor protein having a binding affinity for the viral protein, wherein the viral protein is covalently linked to the receptor protein via a covalent bond between a first amino acid residue on the viral protein, and a second amino acid residue on the receptor protein. In some embodiments, the first amino acid residue and the second residue are each located at a binding interface between the viral protein and the receptor protein. In some embodiments, at least one of the first amino acid residue and the second amino acid residue is not present in a wild-type sequence of the viral protein or the receptor protein. In some embodiments, the first amino acid residue not present in a wild-type sequence of the viral protein and the second amino acid residue not present in a wild-type sequence of the receptor protein. In some embodiments, the first amino acid residue and the second amino acid residue are each cysteine. In some embodiments, the first amino acid residue and the second amino acid residue are each unnatural amino acids. In some embodiments, at least one of the first amino acid residue and the second amino acid residue is an unnatural amino acid.

In some embodiments, the devices and methods disclosed herein utilize an unnatural protein comprising a viral protein and a receptor protein having a binding affinity for the viral protein, wherein the viral protein is covalently linked to the receptor protein via a disulfide bond between a first thiol on the viral protein and a second thiol on the receptor protein. In some embodiments, at least one of the first thiol and the second thiol is part of an amino acid residue not present in a wild-type sequence of the viral protein or the receptor protein. In some embodiments, the first thiol is part of a first amino acid residue not present in a wild-type sequence of the viral protein and the second thiol is part of a second amino acid residue not present in a wild-type sequence of the receptor protein. In some embodiments, the first amino acid residue and the second amino acid residue are each cysteine.

As discussed above, stabilizing the viral protein via a covalent bond between a first amino acid residue on the viral protein and a second amino acid residue on the receptor protein can be used either as (1) an alternative to covalent coupling of the viral protein to the receptor protein via a linker (e.g., fusion protein, PEG-protein conjugate); or (2) in addition to covalent coupling of the viral protein to the receptor protein via a linker.

In the first set of embodiments, the covalent bond between the first amino acid residue and the second amino acid residue is an inter-chain covalent bond. For example, in some embodiments, the covalent bond between the first amino acid residue and the second amino acid residue is an inter-chain disulfide bond. In some embodiments, the viral protein and the receptor protein of a genetic fusion protein are covalently linked by an intrachain disulfide bond (in addition to coupling via a linker). In some embodiments, the disulfide bond is formed between a first cysteine residue on the viral protein and a second cysteine residue on the receptor protein. In some embodiments, at least one of the first cysteine residue and the cysteine residue is not present in a wild-type sequence of the viral protein or the receptor protein. In some embodiments, the first cysteine residue and is not present in a wild-type sequence of the viral protein and the cysteine residue is not present in a wild-type sequence of the receptor protein.

In some embodiments, the covalent bond is between a first amino acid on the viral protein and a second amino acid on the receptor protein. In some embodiments, the first amino acid and the second amino acid are located at the interface between the viral protein and the receptor protein.

In the second set of embodiments, the covalent bond between the first amino acid residue and the second amino acid residue is an intra-chain covalent bond. For example, in some embodiments, the covalent bond between the first amino acid residue and the second amino acid residue is an intra-chain disulfide bond. In some of such embodiments, the unnatural protein will be expressed as a single genetic construct, but the portion comprising the viral protein is cleaved from the portion comprising the receptor protein after translation. For example, an endopeptidase can be used to cleave the two portions of the unnatural protein resulting in two separate polypeptides covalently linked by an interchain covalent bond.

As these approaches are complementary (i.e., covalent coupling of the viral protein to the receptor protein via a linker and covalent coupling of the viral protein to the receptor protein at the interface of the two proteins), many of the features discussed above with respect to unnatural proteins comprising linkers (and other protein constructs comprising a linker), can be applied with equal force to embodiments comprising unnatural proteins comprising a protein staple.

In some embodiments, the viral protein comprises a viral surface protein. In some embodiments, the viral protein comprises a viral coat protein. In some embodiments, the viral protein is an enveloped virus surface protein. In some embodiments, the viral protein is a nonenveloped virus surface protein. In some embodiments, the viral protein is a fragment of a full-length viral protein. In some embodiments, the viral protein is a full-length viral protein. In some embodiments, the fragment of the full-length viral protein comprises a complete domain of a viral protein. In some embodiments, the fragment of the full-length viral protein comprises a portion of a domain of a viral protein. In some embodiments, the fragment of the full-length viral protein is a functional fragment. For example, in some embodiments, the fragments of the full-length viral protein comprises a complete receptor binding motif of the viral protein that retains its ability to bind to the corresponding host cell receptor. In some embodiments, the viral protein comprises at least a portion of a receptor binding domain of the viral surface protein. In some embodiments, the viral surface protein comprises at least a portion of a viral surface protein derived from a respiratory virus.

In some embodiments, the viral protein of the unnatural protein comprises at least a portion of a receptor binding domain of a viral surface protein. In some embodiments, the viral protein is a coronavirus viral surface protein. In some embodiments, the viral protein is an influenza viral surface protein. In some embodiments, the coronavirus viral surface protein is a SARS-CoV2 surface protein. In some embodiments, the coronavirus viral surface protein is a MERS-CoV surface protein.

In some embodiments, the viral protein portion of the unnatural protein comprises at least a portion of a protein selected from the group consisting of an influenza A hemagglutinin protein, an influenza A neuraminidase protein, a MERS-CoV spike protein, a SARS-CoV2 spike protein, a zikavirus E protein, a polyomavirus VP1 protein; an HIV Env protein. In some embodiments, the viral protein comprises at least a portion of a protein selected from the group consisting of an influenza A hemagglutinin protein, an influenza A neuraminidase protein, a MERS-CoV spike protein, and a SARS-CoV2 spike protein. In some embodiments, the viral protein comprises at least a portion of a protein selected from the group consisting of an influenza A hemagglutinin protein, a MERS-CoV spike protein, and a SARS-CoV2 spike protein. In some embodiments, the viral protein comprises at least a portion of a protein selected from the group consisting of a MERS-CoV spike protein, and a SARS-CoV2 spike protein. In some embodiments, the viral protein comprises at least a portion of a SARS-CoV2 spike protein.

In some embodiments, the receptor protein is a fragment of a full-length receptor protein. In some embodiments, the receptor protein is a full-length receptor protein. In some embodiments, the fragment of the full-length receptor protein comprises a complete domain of a receptor protein. In some embodiments, the fragment of the full-length receptor protein comprises a portion of a domain of a receptor protein. In some embodiments, the fragment of the full-length receptor protein is a functional fragment. For example, in some embodiments, the fragments of the full-length receptor protein comprise a complete antigen binding motif of the receptor protein that retains its ability to bind to the cognate antigen on the surface of the pathogen. In some embodiments, the receptor protein comprises at least a portion of an antigen binding domain of the receptor protein.

In some embodiments, the receptor protein portion of the unnatural protein comprises a natural host receptor protein for the viral protein. The natural host receptor protein for the viral protein can be a full-length protein or a portion thereof (e.g., a functional fragment). The protein can comprise at least 50, at least 60, at least 70, at least 80, at least 90, at least 100, at least 150, at least 200, at least 250, at least 300, at least 350, at least 400, at least 450, at least 500, at least 550, at least 600, at least 650, at least 700, at least 750, or at least 805 amino acids of the full-length protein. In some embodiments, the full-length protein is an angiotensin converting enzyme 2 (ACE2).

In some embodiments, the receptor protein comprises at least a portion of a natural binding target for the viral surface protein. In some embodiments, the receptor protein comprises at least a portion of a human protein. In some embodiments, the receptor protein comprises at least a portion of a human cell-surface glycoprotein. In some embodiments, the receptor protein comprises at least a portion of a human cell-surface glycoprotein comprising sialic acid. In some embodiments, the receptor protein comprises at least a portion of an angiotensin converting enzyme 2 (ACE2). In some embodiments, the receptor protein comprises a soluble fragment of ACE2.

In some embodiments, the receptor protein portion of the unnatural protein is coupled to the viral protein via linker. In such embodiments, the covalent bond between the viral protein and the receptor protein is an intra-chain covalent bond. In some embodiments, the covalent bond between the viral protein and the receptor protein is an intra-chain disulfide bond. In some embodiments, the N-terminus of the receptor protein is connected to the C-terminus of the viral protein by a linker. In some embodiments, the linker is a polypeptide linker. In some embodiments, the linker comprises a polypeptide linker. In some embodiments, the linker comprises a water-soluble chemical polymer or oligomer. For example, in some embodiments, the linker comprises a polyethylene glycol

In some embodiments, the receptor protein portion of the unnatural protein is not coupled to the viral protein via linker. In such embodiments, the covalent bond between the viral protein and the receptor protein is an inter-chain covalent bond. In some embodiments, the covalent bond between the viral protein and the receptor protein is an inter-chain disulfide bond.

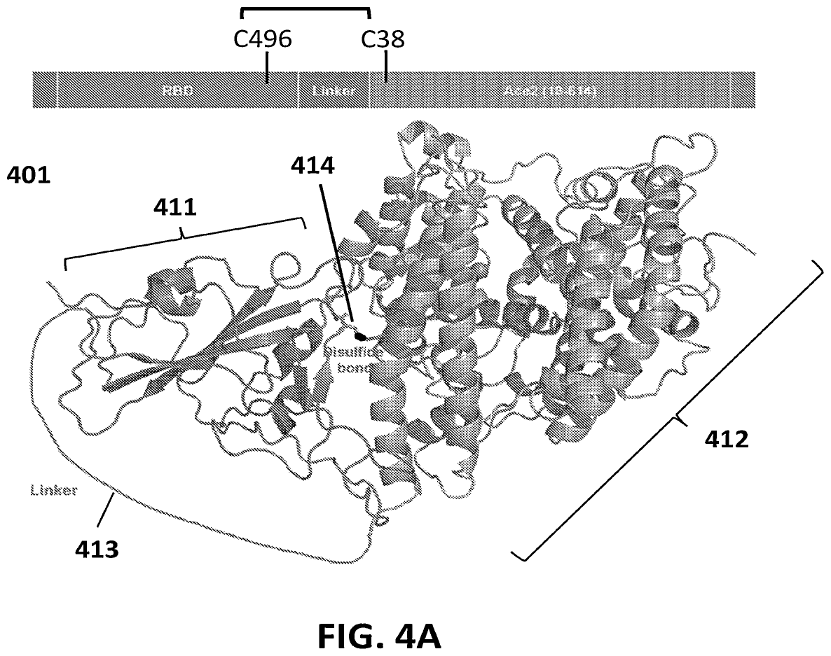

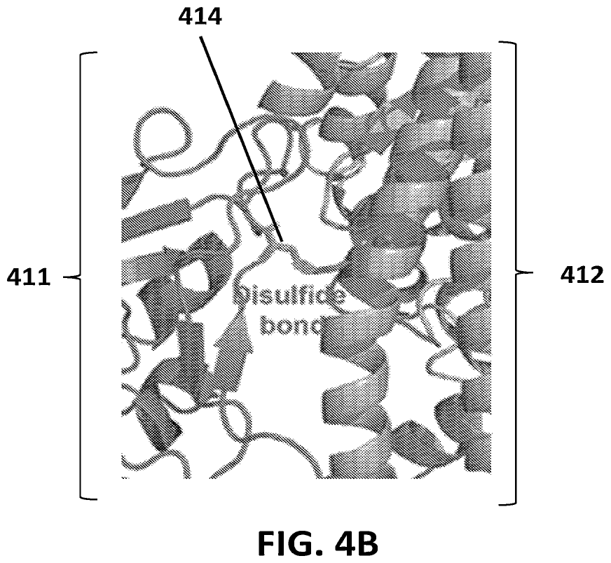

FIG. 4A provides an example of a stapled viral protein/receptor covalent complex 401 of the present disclosure. Stapled protein complex 401 is a fusion protein comprising a portion of SARS-CoV-2 RBD 411 linked to a portion of ACE2 412 via linker 413. The SARS-CoV-2 RBD 411 and ACE2 portion 412 are further covalently linked by disulfide bond 414. Disulfide bond 414 covalently links a first cysteine residue, located on the S-RBD 411 to a second cysteine residue, located on the ACE2 portion 412. These cysteine residues are not present in the native sequence of either S-RBD 411 or ACE2 412.

FIG. 4B is a close-up image of a portion of stapled protein complex 401. Disulfide bond 414 is located at the interface of S-RBD 411 and ACE2 412. In some embodiments, a set of amino acid residues (e.g., one on S-RBD and a second on ACE2) having beta carbons or alpha carbon hydrogens less than 5 angstroms apart are identified and mutated to cysteine.

In some embodiments, the covalent bond between the viral protein and the receptor protein is a disulfide bond formed between a first thiol on a first amino acid residue on the viral protein and a second thiol on a second amino acid residue on the receptor protein (e.g., an interchain or intrachain disulfide bond). In some embodiments, the first thiol is part of a first cysteine that replaces an amino acid residue corresponding to an amino acid residue between positions 142 and 242 of SEQ ID NO: 4. In some embodiments, the second thiol is part of a second cysteine that replaces an amino acid residue corresponding to an amino acid residue between positions 284 and 340 of SEQ ID NO: 4. In some embodiments, the first cysteine replaces an amino acid residue corresponding to position 496 of SEQ ID NO: 1. In some embodiments, the second cysteine residue replaces an amino acid residue corresponding to position 38 of SEQ ID NO: 2. In some embodiments, the first cysteine replaces an amino acid residue corresponding to position 196 of SEQ ID NO:4 and the second cysteine residue replaces an amino acid residue corresponding to position 304 of SEQ ID NO: 4.

In some embodiments, the receptor protein comprises amino acids nos. 284 to 880 of SEQ ID NO:5. In some embodiments, the receptor protein comprises a sequence having, e.g., at least 70%, at least 75%, at least 80%, at least 85%, at least 90%, at least 95%, at least 97%, at least 98%, at least 99% sequence identity (or homology) to amino acids nos. 284 to 880 of SEQ ID NO:5. In some embodiments, the receptor protein comprises a sequence having at least 70% sequence identity (or homology) to amino acids nos. 284 to 880 of SEQ ID NO:5. In some embodiments, the receptor protein comprises a sequence having at least 80% sequence identity (or homology) to amino acids nos. 284 to 880 of SEQ ID NO:5. In some embodiments, the receptor protein comprises a sequence having at least 90% sequence identity (or homology) to amino acids nos. 284 to 880 of SEQ ID NO:5. In some embodiments, the receptor protein comprises a sequence having at least 95% sequence identity (or homology) to amino acids nos. 284 to 880 of SEQ ID NO:5. In some embodiments, the receptor protein comprises a sequence having at least 97% sequence identity (or homology) to amino acids nos. 284 to 880 of SEQ ID NO:5. In some embodiments, the receptor protein comprises a sequence having at least 98% sequence identity (or homology) to amino acids nos. 284 to 880 of SEQ ID NO:5. In some embodiments, the receptor protein comprises a sequence having at least 99% sequence identity (or homology) to amino acids nos. 284 to 880 of SEQ ID NO:5.

In some embodiments, the viral protein comprises amino acids nos. 20 to 242 of SEQ ID NO:5. In some embodiments, the viral protein comprises a sequence having, e.g., at least 70%, at least 75%, at least 80%, at least 85%, at least 90%, at least 95%, at least 97%, at least 98%, at least 99% sequence identity (or homology) to amino acids nos. 20 to 242 of SEQ ID NO:5. In some embodiments, the viral protein comprises a sequence having at least 70% sequence identity (or homology) to amino acids nos. 20 to 242 of SEQ ID NO:5. In some embodiments, the viral protein comprises a sequence having at least 80% sequence identity (or homology) to amino acids nos. 20 to 242 of SEQ ID NO:5. In some embodiments, the viral protein comprises a sequence having at least 90% sequence identity (or homology) to amino acids nos. 20 to 242 of SEQ ID NO:5. In some embodiments, the viral protein comprises a sequence having at least 95% sequence identity (or homology) to amino acids nos. 20 to 242 of SEQ ID NO:5. In some embodiments, the viral protein comprises a sequence having at least 97% sequence identity (or homology) to amino acids nos. 20 to 242 of SEQ ID NO:5. In some embodiments, the viral protein comprises a sequence having at least 98% sequence identity (or homology) to amino acids nos. 20 to 242 of SEQ ID NO:5. In some embodiments, the viral protein comprises a sequence having at least 99% sequence identity (or homology) to amino acids nos. 20 to 242 of SEQ ID NO:5.

In some embodiments, the unnatural protein comprises SEQ ID NO:5. In some embodiments, the unnatural protein comprises a sequence having, e.g., at least 70%, at least 75%, at least 80%, at least 85%, at least 90%, at least 95%, at least 97%, at least 98%, at least 99% sequence identity (or homology) to SEQ ID NO:5. In some embodiments, the unnatural protein consists essentially of SEQ ID NO:5. In some embodiments, the unnatural protein consists essentially of a sequence having, e.g., at least 70%, at least 75%, at least 80%, at least 85%, at least 90%, at least 95%, at least 97%, at least 98%, at least 99% sequence identity (or homology) to SEQ ID NO:5.

In some embodiments, the unnatural protein comprises amino acids nos. 20 to 880 of SEQ ID NO:5. In some embodiments, the unnatural protein comprises a sequence having, e.g., at least 70%, at least 75%, at least 80%, at least 85%, at least 90%, at least 95%, at least 97%, at least 98%, at least 99% sequence identity (or homology) to amino acids nos. 20 to 880 of SEQ ID NO:5. In some embodiments, the unnatural protein comprises a sequence having at least 70% sequence identity (or homology) to amino acids nos. 20 to 880 of SEQ ID NO: 5. In some embodiments, the unnatural protein comprises a sequence having at least 80% sequence identity (or homology) to amino acids nos. 20 to 880 of SEQ ID NO:5. In some embodiments, the unnatural protein comprises a sequence having at least 90% sequence identity (or homology) to amino acids nos. 20 to 880 of SEQ ID NO:5. In some embodiments, the unnatural protein comprises a sequence having at least 95% sequence identity (or homology) to amino acids nos. 20 to 880 of SEQ ID NO:5. In some embodiments, the unnatural protein comprises a sequence having at least 97% sequence identity (or homology) to amino acids nos. 20 to 880 of SEQ ID NO:5. In some embodiments, unnatural protein comprises a sequence having at least 98% sequence identity (or homology) to amino acids nos. 20 to 880 of SEQ ID NO:5. In some embodiments, the unnatural protein comprises a sequence having at least 99% sequence identity (or homology) to amino acids nos. 20 to 880 of SEQ ID NO:5.

### Neutralizing Antibody Detection

Aspects of the present disclosure further provide compositions, systems, and methods for detecting an antibody. The antibody can be a neutralizing antibody. The antibody can be a non-neutralizing antibody. The antibody can be an antibody subpopulation (e.g., a plurality of antibodies specific for a common antigen or pathogen, but comprising different paratopes). For example, the antibody can be neutralizing antibodies or non-neutralizing antibodies for a particular pathogen or antigen.

In some embodiments, the present disclosure provides a method for detecting neutralizing antibodies of a pathogen protein in a biological sample, the method comprising: (a) contacting the biological sample with the pathogen protein, thereby binding a first set of antibodies from the biological sample to the pathogen protein; (b) measuring the first set of antibodies bound to the pathogen protein; (c) contacting the biological sample to a modified form of the pathogen protein, thereby binding a second set of antibodies from the biological sample to the modified form of the pathogen protein; (d) measuring the second set of antibodies bound to the modified form of the pathogen protein; (e) determining a difference between the measurement of the first set of antibodies from step (b) and the measurement of the second set of antibodies from step (d), thereby detecting the neutralizing antibodies in the sample. In some embodiments, two or more of steps (a) through (e) are performed simultaneously. For example, in some embodiments, step (b) and step (d) occur simultaneously.

In some embodiments, the present disclosure provides a method for detecting neutralizing antibodies of a viral protein in a biological sample, the method comprising: (a) contacting the biological sample with the viral protein, thereby binding a first set of antibodies from the biological sample to the viral protein; (b) measuring the first set of antibodies bound to the viral protein; (c) contacting the biological sample to a modified form of the viral protein, thereby binding a second set of antibodies from the biological sample to the modified form of the viral protein; (d) measuring the second set of antibodies bound to the modified form of the viral protein; (e) determining a difference between the measurement of the first set of antibodies from step (b) and the measurement of the second set of antibodies from step (d), thereby detecting the neutralizing antibodies in the sample. In some embodiments, two or more of steps (a) through (e) are performed simultaneously. For example, in some embodiments, step (b) and step (d) occur simultaneously.

In some embodiments, the method utilizes a modified form of the viral protein that possesses diminished affinity for neutralizing antibodies of the viral protein. For example, in some embodiments, the modified form of the viral protein comprises the viral protein covalently coupled to a receptor protein via a linker. In some embodiments, the linker is a polypeptide. In some embodiments, the modified viral protein is a fusion protein that has been genetically fused with the receptor protein. In some embodiments, the modified viral protein comprises a covalent bond between a first amino acid residue on the viral protein and a second amino acid residue on a receptor protein. In some embodiments, the receptor protein has an affinity for the viral protein. For example, in various embodiments, the modified viral protein comprises one or more of the protein conjugates, fusion proteins, unnatural proteins, or stapled proteins disclosed herein.

In some embodiments, the difference between the measurement of the first set of antibodies from step (b) and the measurement of the second set of antibodies form step (d) corresponds to a difference in an abundance of the first set of antibodies and an abundance of the second set of antibodies. For example, in some embodiments, labeled secondary antibodies (e.g., anti-IgG antibodies) are used to determine the abundance of antibodies in each set of antibodies. In some embodiments, the first set antibodies from step (b) will comprise a larger antibody population (and will produce a more intense signal) than the second set antibodies from step (d). In such embodiments, for example, the difference between the measurements of the two sets of antibodies to a difference in an abundance between the two sets of antibodies.

In some embodiments, the difference between the measurement of the first set of antibodies from step (b) and the measurement of the second set of antibodies form step (d) corresponds to a difference in neutralizing antibody abundance between the first set of antibodies and the second set of antibodies. For example, in some embodiments, the first set of antibodies corresponds to both neutralizing antibodies and non-neutralizing antibodies of the viral protein. In some embodiments, the second set of antibodies corresponds to only non-neutralizing antibodies. In such embodiments, for example, the difference between the measurement of the first set of antibodies and the second set of antibodies corresponds to a difference in neutralizing antibodies between the two sets of antibodies.

In some embodiments, wherein step (b) comprises contacting the first set of antibodies and a first set of labeled anti-IgG antibodies and step (d) comprises contacting the second set of antibodies with a second set of labeled anti-IgG antibodies. In some embodiments, the label on either or both of the first set of anti-IgG antibodies and the second set of anti-IgG antibodies comprises a fluorescent label. In some embodiments, the label on both of the first set of anti-IgG antibodies and the second set of anti-IgG antibodies comprises a fluorescent label.

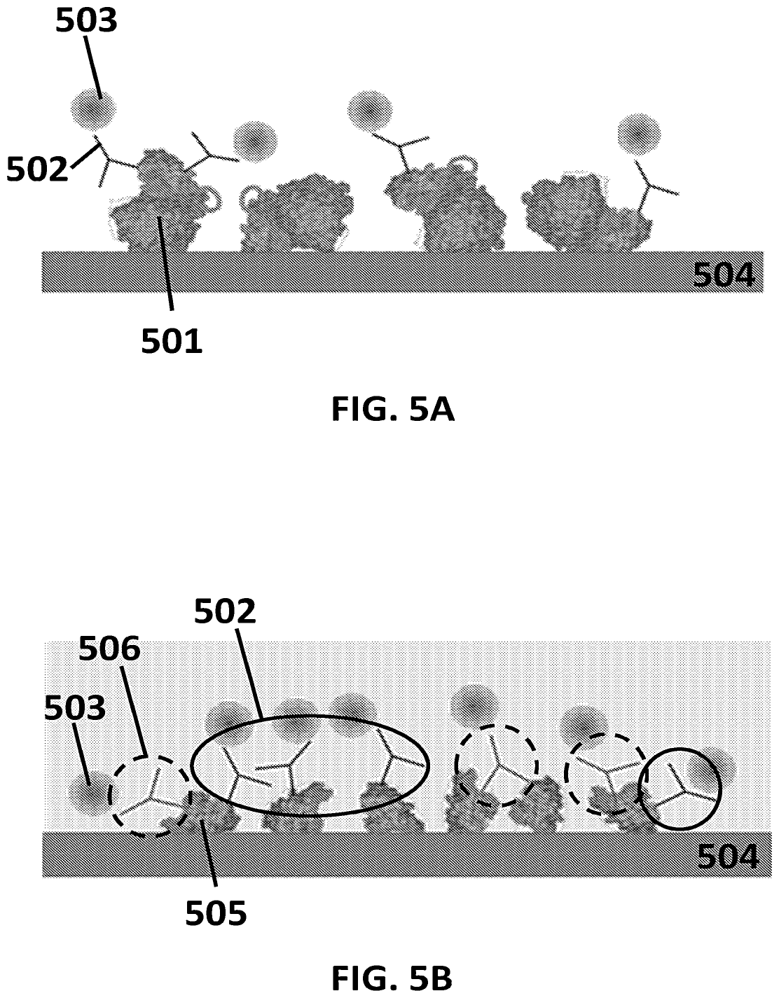

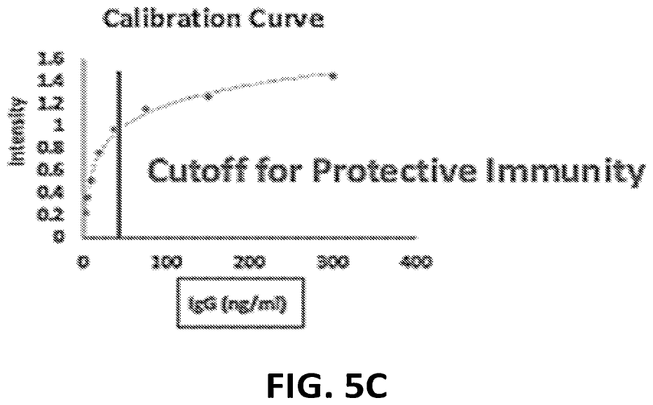

In some embodiments, the method utilizes multiple species for antibody detection. An example of such a format is provided in FIGS. 5A-B, which comprises two species (1) a modified antigen (e.g., SARS-CoV-2 S-protein coupled to an ACE2 receptor) 501 and (2) the unblocked antigen (e.g., SARS-CoV-2 S-protein RBM) 505 coupled to a surface 504 (e.g., the same surface of a single microarray or two separate surfaces). In some embodiments, the two species are coupled to separate locations of the surface to facilitate separate analysis of the species or of molecules coupled thereto.

FIG. 5A outlines a spot comprising an example of modified antigen 501 (e.g., a fusion protein between S-RBD coupled to ACE2). As the COVID-19 S-protein RBM is efficiently blocked by ACE2 in this format, only non-neutralizing antibodies 502 (in this embodiment, antibodies which do not bind the COVID-19 S-protein RBM), bind to the modified antigen 501 of FIG. 5A. For neutralizing antibody capture, a second spot, depicted in FIG. 5B, can comprise a portion of the unblocked antigen 505 (e.g., the COVID-19 S-protein with the RBM unblocked). Unblocked antigen 505 bind a combination of neutralizing antibodies 506 (inscribed with dashed lines) and non-neutralizing antibodies 502 (inscribed with solid lines). Following antibody binding, antibodies coupled to each spot can be detected, for example by adding fluorescently labeled anti-IgG antibodies 503 that bind to both neutralizing 506 and non-neutralizing antibodies 502. The fluorescent label on the anti-IgG antibodies can be detected and/or quantified to detect (1) non-neutralizing antibodies bound to modified antigen 501; and (2) the combination of neutralizing and non-neutralizing antibodies bound to unmasked antigen 505. The difference between these signals can be used to as a measure of neutralizing antibody titer. In some embodiments, a calibration curve of neutralizing antibody concentration is generated, for example, in the format provided in FIG. 5C.

In some embodiments, the unblocked viral protein comprises a viral surface protein. In some embodiments, the viral protein comprises a viral coat protein. In some embodiments, the viral protein is an enveloped virus surface protein. In some embodiments, the viral protein is a nonenveloped virus surface protein. In some embodiments, the viral protein is a viral membrane protein. In some embodiments, the viral protein comprises a receptor binding domain of a viral surface protein. In some embodiments, the viral protein comprises a receptor binding domain of a viral coat protein. In some embodiments, the viral protein comprises a receptor binding domain of a viral envelope protein. In some embodiments, the viral protein comprises a receptor binding domain of a viral membrane protein.

In some embodiments, the viral protein comprises a full-length viral protein. In some embodiments, the viral protein comprises a fragment of a full-length viral protein. In some embodiments, the viral protein comprises at least a portion of a viral protein derived from a virus selected from a coronavirus, an influenza virus, a zikavirus, a polyomoavirus, and an HIV virus. In some embodiments, the viral protein comprises at least a portion of a viral protein derived from a virus selected from a coronavirus and an influenza virus. In some embodiments, the viral protein comprises at least a portion of a viral protein derived from a coronavirus. In some embodiments, the viral protein comprises at least a portion of a viral protein derived from SARS-CoV-2.

In some embodiments, the viral protein comprises a viral protein selected from the group consisting of: an influenza A hemagglutinin protein, an influenza A neuraminidase protein, a MERS-CoV spike protein, a SARS-CoV2 spike protein, a zikavirus E protein, a polyomavirus VP1 protein; an HIV Env protein. In some embodiments, the viral protein comprises a viral protein selected from the group consisting of: an influenza A hemagglutinin protein, an influenza A neuraminidase protein, and a SARS-CoV2 spike protein. In some embodiments, the viral protein comprises a SARS-CoV2 spike protein. In some embodiments, the viral protein comprises a receptor binding domain of a SARS-CoV2 spike protein. In some embodiments, the viral protein comprises a receptor binding motif of a SARS-CoV2 spike protein. In some embodiments, the viral protein comprises amino acids nos. 319 to 541 of SEQ ID NO: 1. In some embodiments, the viral protein comprises a sequence having, e.g., at least 70%, at least 75%, at least 85%, at least 90%, at least 95%, at least 97%, at least 98%, or at least 99% sequence identity (or homology) to amino acids nos. 319 to 541 of SEQ ID NO: 1.

In some embodiments, the modified form of the viral protein comprises at least one of a blocked neutralizing antibody epitope and an altered neutralizing antibody epitope. In some embodiments, the modified form of the viral protein comprises an altered neutralizing antibody epitope. In some embodiments, the modified form of the viral protein comprises an altered neutralizing antibody epitope comprising a mutation, a chemical modification, cleavage, or any combination thereof. In some embodiments, the modified form of the viral protein comprises an altered neutralizing antibody epitope comprising an insertion, a deletion, a truncation or any combination thereof. For example, in some embodiments, neutralizing antibody epitope comprises a chemical modification that allows a covalent bond to be formed between the neutralizing antibody and a receptor protein having an affinity for the viral protein.

In some embodiments, the modified form of the viral protein comprises a blocked neutralizing antibody epitope and a blocking group. In some embodiments, the blocking group comprises a macromolecule selected form the group consisting of a protein, a nucleic acid, or a synthetic polymer. In some embodiments, the blocking group comprises a protein. In some embodiments, the blocking group comprises a receptor protein. In some embodiments, the receptor protein is the cognate receptor for the blocked neutralizing antibody epitope on the viral protein. In some such embodiments, the blocking group comprises an antibody or an epitope-binding fragment thereof. In some embodiments, the blocking group comprises a nucleic acid. In some such embodiments, the blocking group comprises an aptamer or an epitope-binding fragment thereof. In some such embodiments, the blocking group comprises a synthetic polymer. In some embodiments, the blocking group comprises a polyethylene glycol polymer or oligomer.

In some embodiments, the blocking group is covalently coupled to the blocked neutralizing antibody epitope on the viral protein. For example, in some embodiments, the blocking group is covalently coupled to the blocked neutralizing antibody epitope by a disulfide bond. In some embodiments, the disulfide bond is formed between a first cysteine residue on the blocked neutralizing antibody epitope and a second cysteine residue on the blocking group. In some embodiments, the blocking group is covalently coupled to the blocked neutralizing antibody epitope via a 1,3 triazole moiety formed by click chemistry between an azide and an alkyne. In some embodiments, the covalent bond is formed between a first unnatural amino acid residue on the blocked neutralizing antibody epitope and a second unnatural amino acid residue on the blocking group. For example, in some such embodiments, the blocked neutralizing antibody epitope is covalently linked to the blocking group via a triazole between an alkyne-containing unnatural amino acid residue on the blocked neutralizing antibody epitope and an azide-containing unnatural amino acid residue on the blocking group. The reverse configuration is also possible. Further, additional biorthogonal functional groups are known in the art and can be used to form a covalent coupling between the blocked neutralizing antibody epitope and the blocking group.

In some embodiments, the blocking group is covalently coupled to the modified viral protein by a linker. In some embodiments, the blocking group is covalently coupled to the modified viral protein by a polypeptide linker. In some embodiments, the modified viral protein is expressed as a single expression construct comprising the blocked neutralizing antibody epitope, the polypeptide linker, and the blocking group.

In some embodiments, the modified viral protein comprises a viral surface protein. In some embodiments, the modified viral protein comprises a viral coat protein. In some embodiments, the modified viral protein is a viral envelope protein. In some embodiments, the modified viral protein is a viral membrane protein. In some embodiments, the modified viral protein comprises a receptor binding domain of a viral surface protein. In some embodiments, the modified viral protein comprises a receptor binding domain of a viral coat protein. In some embodiments, the modified viral protein comprises a receptor binding domain of a viral envelope protein. In some embodiments, the modified viral protein comprises a receptor binding domain of a viral membrane protein.

In some embodiments, the modified viral protein comprises a full-length viral protein. In some embodiments, the modified viral protein comprises a fragment of a full-length viral protein. In some embodiments, the modified viral protein comprises at least a portion of a viral protein derived from a virus selected from a coronavirus, an influenza virus, a zikavirus, a polyomoavirus, and an HIV virus. In some embodiments, the modified viral protein comprises at least a portion of a viral protein derived from a virus selected from a coronavirus and an influenza virus. In some embodiments, the modified viral protein comprises at least a portion of a viral protein derived from a coronavirus. In some embodiments, the modified viral protein comprises at least a portion of a viral protein derived from SARS-CoV-2.

In some embodiments, the modified viral protein comprises at least a portion of a protein selected from the group consisting of an influenza A hemagglutinin protein, an influenza A neuraminidase protein, a MERS-CoV spike protein, a SARS-CoV2 spike protein, a zikavirus E protein, a polyomavirus VP1 protein; an HIV Env protein. In some embodiments, the modified viral protein comprises at least a portion of a protein selected from the group consisting of an influenza A hemagglutinin protein, an influenza A neuraminidase protein, a MERS-CoV spike protein, and a SARS-CoV2 spike protein. In some embodiments, the modified viral protein comprises at least a portion of a protein selected from the group consisting of an influenza A hemagglutinin protein, a MERS-CoV spike protein, and a SARS-CoV2 spike protein. In some embodiments, the modified viral protein comprises at least a portion of a protein selected from the group consisting of a MERS-CoV spike protein, and a SARS-CoV2 spike protein. In some embodiments, the modified viral protein comprises at least a portion of a SARS-CoV2 spike protein.

In some embodiments, the modified viral protein comprises a coronavirus viral surface protein. In some embodiments, the modified viral protein comprises an influenza viral surface protein. In some embodiments, the coronavirus viral surface protein is a SARS-CoV2 surface protein. In some embodiments, the coronavirus viral surface protein is a MERS-CoV surface protein

In some embodiments, the blocking group comprises a receptor protein. In some embodiments, the receptor protein is a fragment of a full-length receptor protein. In some embodiments, the receptor protein is a full-length receptor protein. In some embodiments, the fragment of the full-length receptor protein comprises a complete domain of a receptor protein. In some embodiments, the fragment of the full-length receptor protein comprises a portion of a domain of a receptor protein. In some embodiments, the fragment of the full-length receptor protein is a functional fragment. For example, in some embodiments, the fragments of the full-length receptor protein comprises a complete antigen binding motif of the receptor protein that retains its ability to bind to the cognate antigen on the surface of the pathogen. In some embodiments, the receptor protein comprises at least a portion of an antigen binding domain of the receptor protein.

In some embodiments, the receptor protein comprises at least a portion of a natural binding target for the viral surface protein. In some embodiments, the receptor protein comprises at least a portion of a human protein. In some embodiments, the receptor protein comprises at least a portion of a human cell-surface glycoprotein. In some embodiments, the receptor protein comprises at least a portion of a human cell-surface glycoprotein comprising sialic acid. In some embodiments, the receptor protein comprises at least a portion of an angiotensin converting enzyme 2 (ACE2). In some embodiments, the receptor protein comprises a soluble fragment of ACE2.

In some embodiments, the receptor protein comprises amino acids nos. 18 to 614 of SEQ ID NO:2. In some embodiments, the receptor protein comprises a sequence having. e.g., at least 70%, at least 75%, at least 80%, at least 85%, at least 90%, at least 95%, 18 to 614 of SEQ ID NO:2. In some embodiments, the receptor protein comprises a sequence having at least 70% sequence identity (or homology) to amino acids nos. 18 to 614 of SEQ ID NO: 2. In some embodiments, the receptor protein comprises a sequence having at least 80% sequence identity (or homology) to amino acids nos. 18 to 614 of SEQ ID NO:2. In some embodiments, the receptor protein comprises a sequence having at least 90% sequence identity (or homology) to amino acids nos. 18 to 614 of SEQ ID NO:2. In some embodiments, the receptor protein comprises a sequence having at least 95% sequence identity (or homology) to amino acids nos. 18 to 614 of SEQ ID NO:2. In some embodiments, the receptor protein comprises a sequence having at least 97% sequence identity (or homology) to amino acids nos. 18 to 614 of SEQ ID NO:2. In some embodiments, the receptor protein comprises a sequence having at least 98% sequence identity (or homology) to amino acids nos. 18 to 614 of SEQ ID NO:2. In some embodiments, the receptor protein comprises a sequence having at least 99% sequence identity (or homology) to amino acids nos. 18 to 614 of SEQ ID NO:2.

In some embodiments, the modified viral protein comprises SEQ ID NO:4. In some embodiments, the modified viral protein comprises a sequence having. e.g., at least 70%, at least 75%, at least 80%, at least 85%, at least 90%, at least 95%, at least 97%, at least 98%, at least 99% sequence identity (or homology) to SEQ ID NO:4. In some embodiments, the modified viral protein consists essentially of SEQ ID NO:4. In some embodiments, the modified viral protein consists essentially of a sequence having, e.g., at least 70%, at least 75%, at least 80%, at least 85%, at least 90%, at least 95%, at least 97%, at least 98%, at least 99% sequence identity (or homology) to SEQ ID NO:4.

In some embodiments, the modified viral protein comprises amino acids nos. 20 to 880 of SEQ ID NO:4. In some embodiments, the modified viral protein comprises a sequence having, e.g., at least 70%, at least 75%, at least 80%, at least 85%, at least 90%, at least 95%, at least 97%, at least 98%, at least 99% sequence identity (or homology) to amino acids nos. 20 to 880 of SEQ ID NO:4. In some embodiments, the modified viral protein comprises a sequence having at least 70% sequence identity (or homology) to amino acids nos. 20 to 880 of SEQ ID NO:4. In some embodiments, the modified viral protein comprises a sequence having at least 80% sequence identity (or homology) to amino acids nos. 20 to 880 of SEQ ID NO:4. In some embodiments, the modified viral protein comprises a sequence having at least 90% sequence identity (or homology) to amino acids nos. 20 to 880 of SEQ ID NO: 4. In some embodiments, the modified viral protein comprises a sequence having at least 95% sequence identity (or homology) to amino acids nos. 20 to 880 of SEQ ID NO:4. In some embodiments, the modified viral protein comprises a sequence having at least 97% sequence identity (or homology) to amino acids nos. 20 to 880 of SEQ ID NO:4. In some embodiments, modified viral protein comprises a sequence having at least 98% sequence identity (or homology) to amino acids nos. 20 to 880 of SEQ ID NO:4. In some embodiments, the modified viral protein comprises a sequence having at least 99% sequence identity (or homology) to amino acids nos. 20 to 880 of SEQ ID NO:4.

In some embodiments, the modified viral protein comprises SEQ ID NO:5. In some embodiments, the modified viral protein comprises a sequence having, e.g., at least 70%, at least 75%, at least 80%, at least 85%, at least 90%, at least 95%, at least 97%, at least 98%, at least 99% sequence identity (or homology) to SEQ ID NO:5. In some embodiments, the modified viral protein consists essentially of SEQ ID NO:5. In some embodiments, the modified viral protein consists essentially of a sequence having, e.g., at least 70%, at least 75%, at least 80%, at least 85%, at least 90%, at least 95%, at least 97%, at least 98%, at least 99% sequence identity (or homology) to SEQ ID NO:5.

In some embodiments, the modified viral protein comprises amino acids nos. 20 to 880 of SEQ ID NO:5. In some embodiments, the modified viral protein comprises a sequence having, e.g., at least 70%, at least 75%, at least 80%, at least 85%, at least 90%, at least 95%, at least 97%, at least 98%, at least 99% sequence identity (or homology) to amino acids nos. 20 to 880 of SEQ ID NO:5. In some embodiments, the modified viral protein comprises a sequence having at least 70% sequence identity (or homology) to amino acids nos. 20 to 880 of SEQ ID NO:5. In some embodiments, the modified viral protein comprises a sequence having at least 80% sequence identity (or homology) to amino acids nos. 20 to 880 of SEQ ID NO:5. In some embodiments, the modified viral protein comprises a sequence having at least 90% sequence identity (or homology) to amino acids nos. 20 to 880 of SEQ ID NO: 5. In some embodiments, the modified viral protein comprises a sequence having at least 95% sequence identity (or homology) to amino acids nos. 20 to 880 of SEQ ID NO:5. In some embodiments, the modified viral protein comprises a sequence having at least 97% sequence identity (or homology) to amino acids nos. 20 to 880 of SEQ ID NO:5. In some embodiments, modified viral protein comprises a sequence having at least 98% sequence identity (or homology) to amino acids nos. 20 to 880 of SEQ ID NO:5. In some embodiments, the modified viral protein comprises a sequence having at least 99% sequence identity (or homology) to amino acids nos. 20 to 880 of SEQ ID NO:5.

In some embodiments, the viral protein of (a) and the modified form of the viral protein of (c) are provided on a microarray. In some embodiments, the viral protein of (a) is provided on a first set of discrete regions on a microarray and the modified form of the viral protein of (c) is provided on a second set of discrete regions on the microarray. The first set of discrete regions does not overlap from the second set of discrete regions, such that the signals from step (b) can be spatially resolved form the signals from step (d).

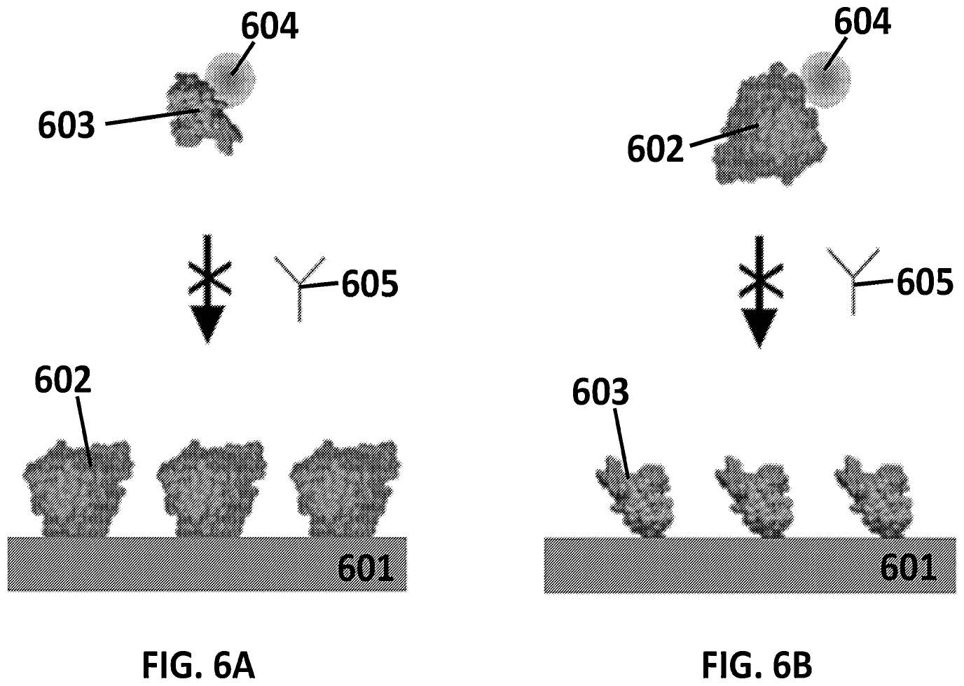

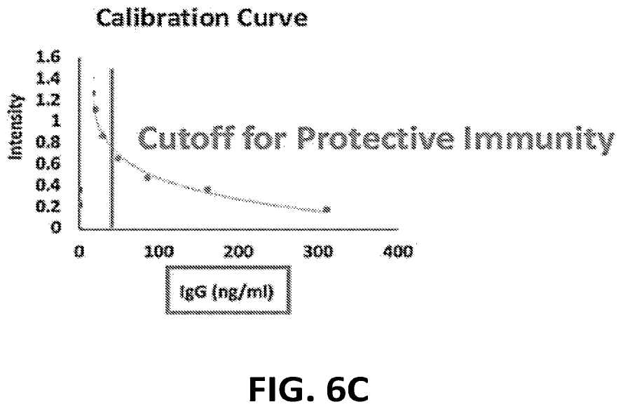

FIGS. 6A and 6B provide alternative formats for neutralizing antibody detection. FIG. 6A depicts a substrate 601 comprising a receptor 602 for a viral protein 603. In some embodiments, the viral protein 603 is be provided in a soluble form in which it is capable of binding to the substrate-bound receptor 602. In some embodiments, the viral protein 603 comprises a detectable label 604, such as a fluorophore, which enables quantitation of viral protein 603 binding to a spot containing the substrate-bound receptor 602. In some embodiments, to determine neutralizing antibody titer (e.g., an amount or concentration), a method comprises contacting the viral protein 603 to the substrate 601 in the presence of an antibody 605 or antibody repertoire (e.g., a mixture containing multiple antibodies). In such cases, the presence of a neutralizing antibody for the viral protein 603 can inhibit its binding to the substrate-bound receptor 602, such that a degree to which viral protein 603 binding is diminished serve as a measure of neutralizing antibody titer. In some embodiments, the receptor 602 and the viral protein 603 each have, for example, a cysteine residue that forms a disulfide bond between receptor 602 and viral protein 603, thereby covalently linking the two binding partners. Such embodiments can for example, be utilized for the detection of neutralizing antibodies as described elsewhere in the present disclosure. FIG. 6B depicts an alternative format to FIG. 6A, in which the viral protein 603 is bound to the substrate 601, and the receptor protein 602 is provided in soluble form and coupled to the detectable label 604. FIG. 6C presents neutralizing antibody data generated with formats of FIGS. 6A-B, in which increased neutralizing antibody titer (x-axis) diminishes signal intensity (y-axis) generated at a spot with substrate-bound receptor 602 or viral 603 proteins.

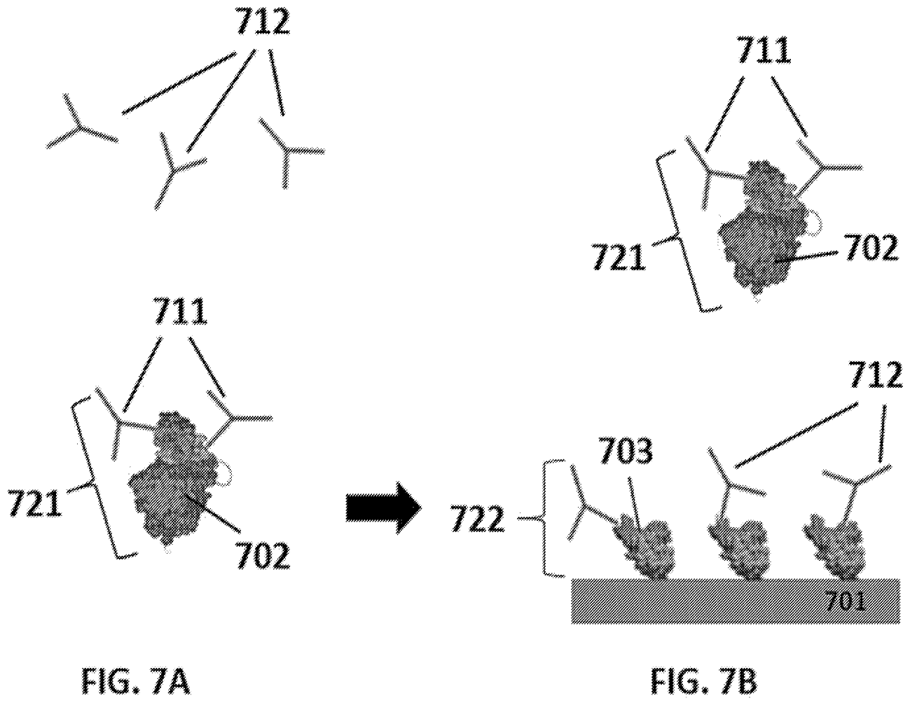

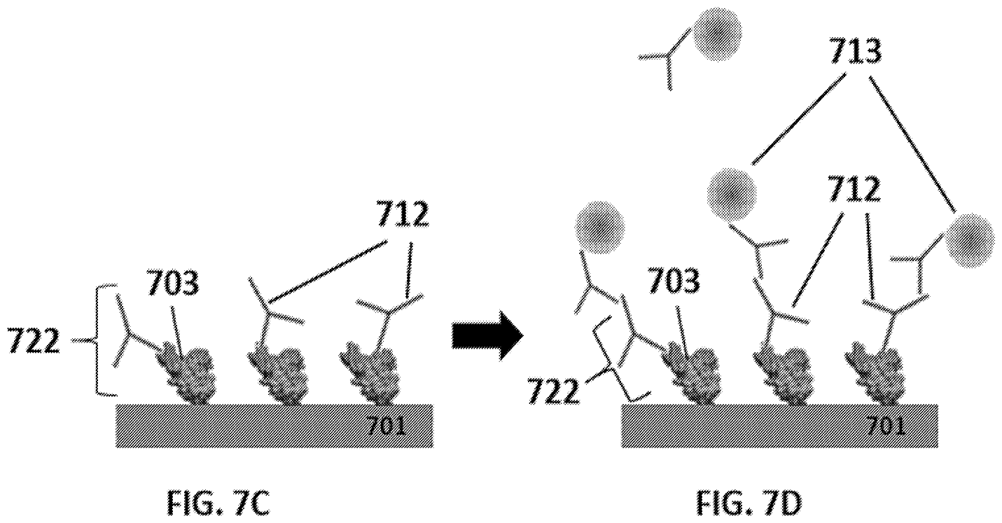

FIGS. 7A-7D provide another embodiment of the present disclosure for detecting neutralizing antibodies (i.e., antibodies that bind to and neutralize viral protein 703). FIG. 7A shows the first step of this method. As shown in FIG. 7A, a biological sample comprising a mixture of a first set of antibodies (i.e., non-neutralizing antibodies) 711 and a second set of antibodies (i.e., neutralizing antibodies) 712. The biological sample is mixed with a solution comprising a modified form of a viral protein 702, thereby bringing the first set of antibodies 712 in contact with the modified form of the viral protein 702 and forming a first antibody-protein complex 721 (i.e., a complex between non-neutralizing antibodies 711 and modified viral protein 702). In some embodiment, during this first step, a sufficient amount of modified protein 702 is present to bind substantially all of the first set of antibodies 711 in the biological sample, thereby sequestering these antibodies in the first antibody-protein complex 721. In some cases, the modified form of the viral protein is not immobilized on a surface.

FIG. 7B shows the second step of the method, in which the biological sample comprises the first antibody-protein complex 721 (i.e., the first set of antibodies 711 bound to the modified viral protein 702) and the second set of antibodies 712, which remains unbound. The biological sample is then contacted with a surface 701, onto which an unmodified viral protein 703 is immobilized. Upon contacting the biological sample with surface 701, the second set of antibodies 712 binds to the unmodified protein 703, thereby forming a second antibody-protein complex 722. Thus, while the first antibody-protein complex 721 remains in solution, the second antibody-protein complex 722 is immobilized on surface 701.

FIG. 7C shows the third steps of the method. In the third step, the first set of antibodies 711 is separated from the second set of antibodies 712. For example, in some embodiments, the solution comprising the biological sample is removed from the surface 701 (e.g., washed away), thereby removing the first antibody-protein complex 721 and leaving the second antibody-protein complex 722 immobilized on the surface 701. In this manner, the first set of antibodies (i.e., non-neutralizing antibodies) 711 is separated from the second set of antibodies (i.e., neutralizing antibodies) 712.

FIG. 7D shows the last step of the method. In FIG. 7D, labeled anti-IgG antibodies 713 are brought in contact with the second antibody-protein complex 722 (comprising second set of antibodies 711 bound to unmodified viral protein 703), which is immobilized on the surface 701. These labeled anti-IgG antibodies 713 bind to the neutralizing antibodies 713, sequestered in the second protein-antibody complex 722. Any excess labelled anti-IgG antibodies 713 are then washed away from the surface 701, and the label (e.g., fluorescence label) is then detected. In some embodiments, detection and/or quantification of the label on the anti-IgG antibodies 713 bound to the immobilized second antibody-protein complex 722 allows for detection and/or quantification of the neutralizing antibodies 712 present in the biological sample.

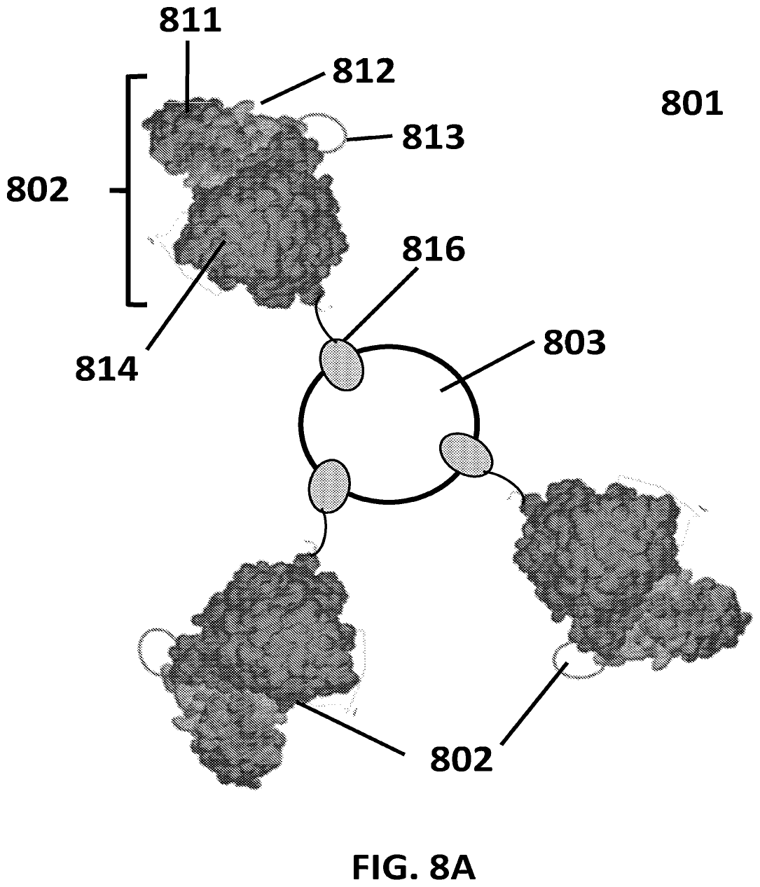

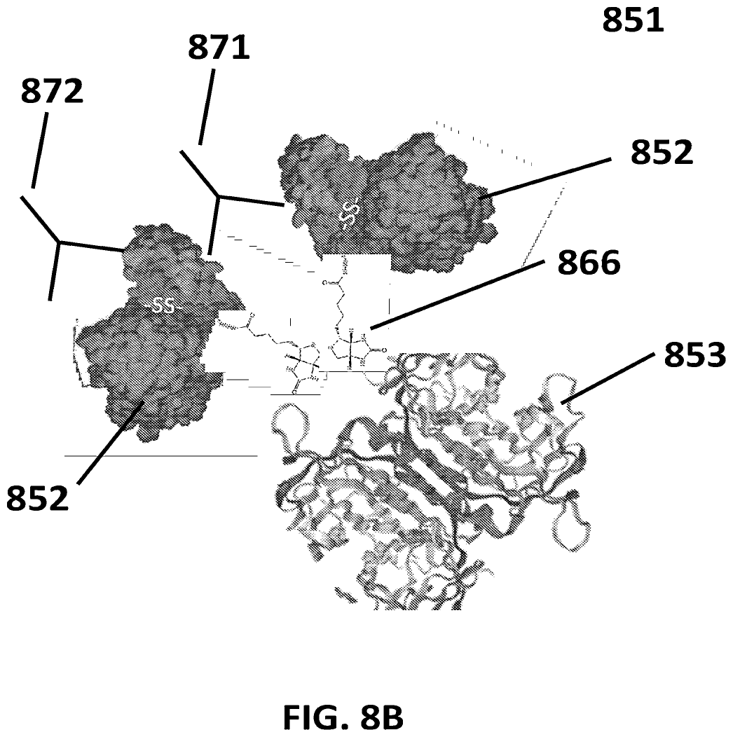

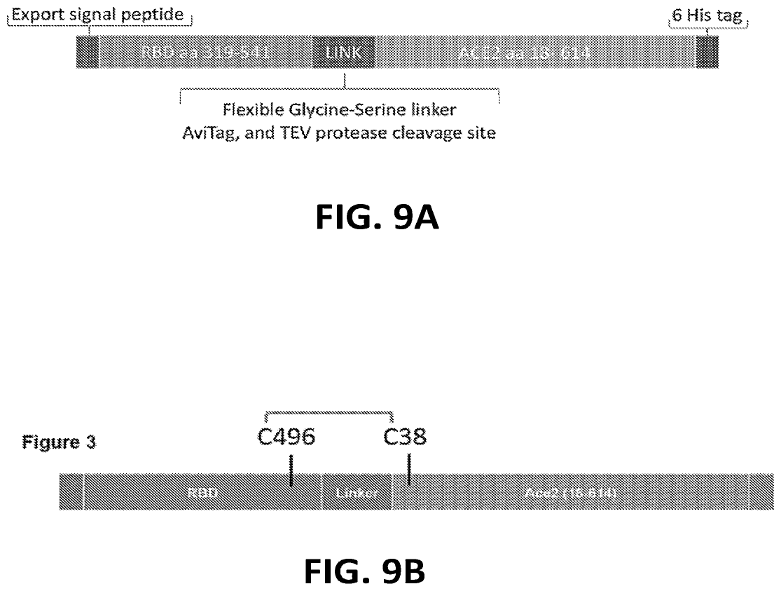

In some embodiments, the methods of the present disclosure utilize a multi-valent fusion protein complex (e.g., a covalent or non-covalent complex comprising two or more modified viral proteins). FIG. 8A provides one example of such a multivalent modified protein complex 801. Multivalent modified protein complex 801 comprises a plurality of modified proteins 802 of the present disclosure (e.g., a fusion protein and/or stapled protein of the present disclosure) attached to one or more scaffolds 803. In some embodiments, the scaffold is, for example, a protein (e.g., neutravidin) or a polymer (e.g., dextran, PEG, etc.) configured to bind the plurality of modified proteins 802. In the exemplary embodiment provided in FIG. 8A, each modified protein comprises a viral surface protein 814 (e.g., intact viral surface protein or fragment thereof), a blocking group 811 (e.g., a receptor protein or portion thereof) that blocks the solvent-exposed surface of the receptor binding motif 812 of the viral surface protein 814. In some embodiments, the viral surface protein 814 is attached to the blocking group 811 via a first linker 813 (e.g., a polypeptide linker). For the embodiment set forth in FIG. 8A, the modified protein 802 is attached to the scaffold 803 via a second liner 816 (e.g., a linker comprising a polypeptide or an organic polymer). Further, in various embodiments, the second linker 816 can be attached to the scaffold 803 through a covalent bond and/or a non-covalent interaction.

FIG. 8B provides one example of such a multivalent modified protein complex 851. In this exemplary embodiment, the scaffold comprises neutravidin 853, which is bound to two modified proteins 852 (e.g., fusion protein and/or stapled protein of the present disclosure) via two biotin molecules 866 covalently attached to the modified proteins 852. As shown in FIG. 8B, multivalent protein complex 851 can bind to non-neutralizing antibodies in several binding modes. For example, non-neutralizing antibody 871 can simultaneously bind to two modified proteins 852 due to their close proximity mediated by binding to neutravidin 853, while non-neutralizing antibody 872 binds to only one modified protein 852.

Many multivalent protein complexes are envisioned by the present disclosure. For example, in some embodiments, there are, e.g., two or more modified proteins 802 attached (covalently and/or non-covalently) to the scaffold molecule 803. In some embodiments, the multivalent protein complex comprises an average of, e.g., 2, 3, 4, 5, 6, 7, 8, 9 10, 11, or 12 modified proteins 802 connected to one scaffold molecule 803. In some embodiments, the multivalent protein complex comprises between 2 and 5 modified proteins bound to the scaffold molecules. In some embodiments, the multivalent protein complex comprises, e.g., between 2 and 10, between 2 and 9, between 2 and 8, between 2 and 7, between 2 and 6, between 2 and 5, between 2 and 4, or between 2 and 3 modified proteins bound to the scaffold molecules. In some embodiments, the multivalent protein complex comprises, e.g., between 3 and 10, between 3 and 9, between 3 and 8, between 3 and 7, between 3 and 6, between 3 and 5, or between 3 and 4 modified proteins bound to the scaffold molecules.

In some embodiments, the multivalent protein complex comprises (i) a plurality of modified proteins and (ii) a scaffold, wherein the scaffold is attached to each of the plurality of modified proteins. In some embodiments, the multivalent protein complex comprises a plurality of modified protein attached to a scaffold via a covalent attachment. In some embodiments, the multivalent protein complex comprises a plurality of modified protein attached to a scaffold via a non-covalent interaction.

In some embodiments, the multivalent protein complex comprises a plurality of modified proteins that are coupled to a multimerizing domain (e.g., through a polypeptide linker). In some embodiments, the multimerizing domain comprises an amino acid sequence. In some embodiments, the multimerizing domain comprises a T4 trimerization domain.

In various embodiments, the modified protein comprises any modified protein disclosed herein (e.g., fusion protein and/or stapled protein, etc.), or any combination thereof.

In some embodiments, the scaffold comprises a biomolecule. In some embodiments, the scaffold comprises a water-soluble polymer. In some embodiments, the scaffold comprises a protein. In some embodiments, the scaffold comprises a biotin-binding protein. In some embodiments, the scaffold comprises neutravidin.

In some embodiments, each of the plurality of modified proteins is attached to the scaffold via a linker (e.g. a linker comprising a water-soluble oligomer, an alkylene chain, etc.). In some embodiments, two or more modified proteins are attached to the scaffold via a single linker (e.g., a branched linker). In some embodiments, the linker comprises biotin.

In some embodiments, each of the plurality of modified proteins is attached to the scaffold via an attachment comprising a covalent attachment and/or a noncovalent interaction. In some embodiments, each of the plurality of modified proteins is attached to the scaffold via an attachment comprising a noncovalent interaction. In some embodiments, the linker comprises biotin and the biotin of the linker binds to the scaffold via a noncovalent interaction.

In some embodiments, each of the plurality of modified proteins is attached to the scaffold via an attachment comprising a covalent attachment. In some embodiments, the covalent attachment comprises a click chemistry product. In some embodiments, the covalent attachment comprises, for example, a triazole, a disulfide, an alpha-substituted carbonyl (e.g., reaction product of a nucleophile to an alpha-halo acetamide, a maleimide, etc.), a tetrazine, etc.

Numerous variations, changes, and substitutions may occur to those skilled in the art without departing from the present disclosure. For example, for ease of understanding, the preceding methods of detecting neutralizing antibodies (e.g., methods set forth in FIGS. 5A-7D) are described and claimed with reference to, for example, “a viral protein”, “an unmodified viral protein”, and/or “a modified form of the viral protein.” However, it will be clear to those of skill in the art that such references include methods in which a plurality of the viral proteins and/or modified viral proteins are used, all of which are within the scope of the present disclosure and claims. Further, it will be understood that references in the specification and claims to a “set of antibodies” binding to a particular protein (e.g., a viral protein or a modified viral protein) shall be understood to include situations in which a single antibody binds to a single viral protein and/or a single antibody binds to two viral proteins simultaneously, as well as situations in which a population of antibodies binds to a population of viral proteins (e.g., in a 1:1 ratio, less than 1:1 ratio, or greater than 1:1 ratio).

In some aspects, the present disclosure provides methods for qualitatively and/or quantitatively assessing the immunity profile of an individual using a blood sample (e.g., whole blood, blood plasma, venous whole blood, fingerstick whole blood, etc.), as well as using that immunity profile to predict the duration of an individual's current immunity, predict a time for administering a vaccine dose, and/or determine whether to administer to an individual anti-viral medication. In some embodiments, the present disclosure provides a cell-free test for quantitative measurement of neutralizing activity against SARS-CoV-2 and its variants. In some embodiments, the cell free test can quantify neutralizing antibodies.

In one aspect, the present disclosure provides a method for measuring NAb levels in an individual, the method comprising:
(a) obtaining a sample taken from the subject;
(b) measuring a concentration of neutralizing antibodies in the sample using a cell-free method; wherein step (b) is completed within one hour.

In some embodiments, step (b) is completed within two hours. In some embodiments, step (b) is completed within 90 minutes. In some embodiments, step (b) is completed within 75 minutes. In some embodiments, step (b) is completed within 60 minutes. In some embodiments, step (b) is completed within 45 minutes. In some embodiments, step (b) is completed within 30 minutes. In some embodiments, step (b) is completed within 20 minutes. In some embodiments, step (b) is completed within 15 minutes.

In one aspect, the present disclosure provides a method for measuring NAb levels in an individual, the method comprising:
(a) obtaining a sample taken from the subject;
(b) adding to the sample an agent that selectively non-neutralizing antibodies
(c) measuring a concentration of neutralizing antibodies in the sample using a cell-free method; wherein step (c) is completed within one hour.

In some embodiments, step (c) is completed within two hours. In some embodiments, step (c) is completed within 90 minutes. In some embodiments, step (c) is completed within 75 minutes. In some embodiments, step (c) is completed within 60 minutes. In some embodiments, step (c) is completed within 45 minutes. In some embodiments, step (c) is completed within 30 minutes. In some embodiments, step (c) is completed within 20 minutes. In some embodiments, step (c) is completed within 15 minutes.

In some embodiments, measuring a concentration of neutralizing antibodies in the sample utilizes a method described herein (e.g., those described with respect to FIGS. 5A-7D). Further, any methods described herein can be multiplexed (e.g., a plurality of antibodies populations simultaneously detected). In some embodiments, at least two populations of neutralizing antibodies (e.g., NAbs directed at different proteins or different protein variants) are detected simultaneously. In some embodiments, at least three populations of neutralizing antibodies (e.g., NAbs directed at different proteins or different protein variants) are detected simultaneously. In some embodiments, this multiplexing is achieved by spotting each of the plurality of different antigens on a different position (e.g., spot or well) on a surface.

In one aspect, the present disclosure provides a method for predicting an immune response to a viral pathogen in a subject, the method comprising:
(a) obtaining a sample taken from the subject;
(b) quantifying an observed level of neutralizing antibodies in the sample using a cell-free bioassay procedure comprising:
   (i) adding to the sample a first agent that selectively binds non-neutralizing antibodies against the viral pathogen thereby generating a pretreated sample; and
   (ii) contacting the pretreated sample with a second agent that binds neutralizing antibodies against the viral pathogen; and
   (iii) detecting the neutralizing antibodies to the second agent; and
(c) comparing the observed level of neutralizing antibodies to a predetermined threshold level of neutralizing antibodies.

In another aspect, the present disclosure provides a method for immunizing a subject against a viral pathogen, the method comprising:
(a) obtaining a sample taken from the subject;
(b) quantifying an observed level of neutralizing antibodies in the sample using a cell-free bioassay;
(c) comparing the observed level of neutralizing antibodies to a predetermined threshold level of neutralizing antibodies; and
(d) if the observed level of neutralizing antibodies in the sample is lower than the predetermined threshold level of neutralizing antibodies, then administering a vaccine directed against the viral pathogen.

In some aspects, the present disclosure provides a method for treating a subject infected with a viral pathogen, the method comprising:
(a) obtaining a sample taken from the subject;
(b) quantifying an observed level of neutralizing antibodies in the sample using a cell-free bioassay;
(c) comparing the observed level of neutralizing antibodies to a predetermined threshold level of neutralizing antibodies; and
(d) if the observed level of neutralizing antibodies in the sample is lower than the predetermined threshold level of neutralizing antibodies, then administering an antiviral therapy directed against the viral pathogen.

In some embodiments, the quantifying an observed level of neutralizing antibodies in the blood sample utilizes a method described herein (e.g., those described with respect to FIGS. 5A-7D).

In some embodiments, the sample comprises a blood sample. In some embodiments, the blood sample comprises whole blood (e.g., fingerstick blood, venous whole blood, etc.). In some embodiments, the blood sample comprises fingerstick blood. In some embodiments, the sample comprises a blood fraction (e.g., blood plasma). In some embodiments, the sample comprises blood plasma.

In some embodiments, the sample comprises, e.g., less than 1 mL, less than 0.9 mL, less than 0.8 mL, less than 0.7 mL, less than 0.6 mL, less than 0.5 mL, less than 0.4 mL, less than 0.3 mL, less than 0.2 mL, or less than 0.1 mL of blood. In some embodiments, the sample comprises, e.g., between 5 μL and 1 mL, between 5 μL and 0.9 mL, between 5 μL and 0.8 mL, between 5 μL and 0.7 mL, between 5 μL and 0.6 mL, between 5 μL and 0.5 mL, between 5 μL and 0.4 mL, between 5 μL and 0.3 mL, between 5 μL and 0.2 mL, or between 5 μL and 0.1 mL of blood. In some embodiments, the sample comprises, e.g., between 5 μL and 100 μL, between 5 μL and 90 μL, between 5 μL and 80 μL, between 5 μL and 70 μL, between 5 μL and 60 μL, between 5 μL and 50 μL, between 5 μL and 40 μL, between 5 μL and 30 μL, between 5 μL and 20 μL, or between 5 μL and 10 μL of blood. In some embodiments, the sample comprises, e.g., between 15 μL and 100 μL, between 15 μL and 90 μL, between 15 μL and 80 μL, between 15 μL and 70 μL, between 15 μL and 60 μL, between 15 μL and 50 μL, between 15 μL and 40 μL, or between 15 μL and 30 μL of blood.

While various embodiments of the present disclosure have been shown and described herein, it will be obvious to those skilled in the art that such embodiments are provided by way of example only. Numerous variations, changes, and substitutions may occur to those skilled in the art without departing from the present disclosure. Thus, it should be understood that various alternatives to the embodiments of the present disclosure described herein may be employed.

### EXAMPLES

The following examples are illustrative and non-limiting to the scope of the compositions, devices, and methods disclosed herein.

### Protein Design and Expression

Baculovirus expression. Recombinant baculovirus was generated by using a Bac-to-Bac expression system (Invitrogen) according to the manufacture's instruction. Briefly, the RBD/ACE2 fusion protein (3A, RBD-AviTag-ACE2-6×HisTag) was cloned into pFastBac vector, which was transformed into competent E. coli (One Shot March1). This donor plasmid was transformed into DH10Bac competent E. coli to construct a recombinant bacmid (baculovirus shuttle vector) via site-specific transposition. The bacmid DNA were isolated and used to transfect ExpiSf9™ cells grown in ExpiSf CD Medium (Gibco). The secreted RBD/ACE2 fusion protein (3A) in ExpiSf9 cell supernatant was collected and purified using Tangential flow filtration (Pall Corporation) and Ni-NTA affinity column (HisTrap HP. Cytivia) (Kurasawa et al., 2020).

Transient Mammalian Expression. The DNA encoding the RBD and RBD/ACE2 fusion proteins were inserted in mammalian-based expression vector, pcDNA3.4 (Purchased from Life science market), for transient protein expression in suspension-based mammalian system. The vector has full length human cytomegalovirus (CMV) immediate-early promoter for high level gene expression together with the woodchuck posttranscriptional regulatory element (WPRE) downstream of the cloning site to enhance protein expression (Fang et al., 2017). Constructs were designed with Kozak sequence preceding the start codon, for ensuring the correct translation initiation, followed by a signal peptide (mouse Ig heavy chain variable region) for protein secretion (Korn et al., 2020). Immediately after the signal peptide. DNA encoding RBD (Spike protein amino acids 319-541) was inserted. For the fusion protein constructs, the C-terminus of RBD was fused with amino acid 18 of Ace-2 by a linker sequence that consists of Avi-tag for enzymatic biotinylation. Tobacco Etch Virus (TEV) protease site for cleavage and a short flexible (Gly4Serine)2 linker (SEQ ID NO: 7) to allow unconstrained RBM/ACE2 interface interactions. At the C-terminus of Ace-2 (ectodomain truncated at amino acid 614 to ablate dimerization domain) a 6×Histidine tag (SEQ ID NO: 8) was added to aid in Ni-NTA affinity purification (FIG. 9A). All the gene sequences were optimized for human system using online codon optimization tools and synthesized by IDT (https://www.idtdna.com/).

Engineering a disulfide bond in RBM/ACE2 interface. The atomic model of ACE2 and SARS-CoV-2 RBD (RBD) pdb code 7C8D (https://www.rcsb.org/structure/7C8D) was examined for close contacts between ACE2 and the RBD. Coot version 0.9.6 (https://www2.mrc-lmb.cam.ac.uk/personal/pemsley/coot/) was used to identify possible cysteine bonds following the protocol outlined in Noeris et. al 2014. Briefly. All ACE2 and RBD amino acids with beta carbons or alpha carbon hydrogens less than 5 angstroms apart were identified and mutated to cysteines. The cysteine rotamer that minimized the distance between cysteine side chains without steric clash was chosen. Ace2 residue 38 (Asp) and RBD residue 496 (Gly) were identified as likely candidates for disulfide bond formation (1.7 angstrom S-S distance and 91 degree dihedral angle). This candidate cysteine double mutant was incorporated into the protein depicted by SEQ ID NO:5 and the resulting staple fusion protein used for neutralizing antibody detection, as described below.

Design and structure of Fusion Constructs. As shown in FIG. 9B, the genetic construct comprises signal peptide (far left) followed by RBD (left-middle), linker sequence (middle), Ace2 (right-middle), and 6×His (SEQ ID NO: 8) (far right). Amino acid numbering is according to location in polypeptides as expressed in original biological context e.g. RBD numbering is in the context of the spike protein of the Wuhan SARS-Cov2 virus. Ace2 residue 38 (Asp) and RBD residue 496 (Gly) were each mutated to cysteine residues.

Biotinylation and streptavidin gel-shift assay. Proteins were biotinylated using in vitro biotinylation kit (BirA500, Avidity). After biotinylation at 30° C. for 40 min, free biotin was removed using gel filtration (size exclusive chromatography). The extent of biotinylating protein was confirmed using streptavidin gel-shift assay (Fairhead M. and Howarth M. (2015)).

Table 2 provides an exemplary list of proteins used to validate the methods herein. B-M5B is a biotinylated SARS-COV-2 Receptor Binding Domain from the original SARS-CoV-2 strain. It comprises an AviTag region for immobilization and a hexahistidine tag for purification. B-14B is a biotinylated SARS-COV-2 Receptor Binding Domain from the delta variant of SARS-CoV-2. It comprises an AviTag region for immobilization and a hexahistidine tag for purification. B-3A and B-M3D4 are each fusion protein comprising the RBD of SARS-CoV-2, the ACE2 ectodomain, and a linker comprising a TEV endopeptidase sequence and polyglycine regions. Each of these fusion proteins comprise an AviTag region for immobilization and a hexahistidine tag for purification. For B-M3D4, Ace2 residue 38 (Asp) and RBD residue 496 (Gly) were each mutated to cysteine residues, which formed an intrachain disulfide bond.

TABLE 2

| | | | Molecular | | | SEQ |
|---|---|---|---|---|---|---|
| | Description | | weight | | Expression | ID NO: |
| B-M5B | Biotinylated SARS | RBD-AviTag- | 28 | kDa | Mammalian | — |
| | CoV2 RBD | 6XHis | | | Expi293 | |
| | Wuhan (319-541) | | | | cells | |
| B-14B | Biotinylated SARS | RBD*¹-AviTag- | 28 | kDa | Mammalian | — |
| | CoV2 RBD delta | 6XHis | | | Expi293 | |
| | variant*¹ (319-541) | | | | cells | |
| B-3A | Biotinylated SARS | RBD-Linker-TEV- | 114 | kDa | Baculovirus | 3 |
| | CoV2 RBD (319- | Linker-ACE2- | | | Sf9 cells | |
| | 541) and ACE2 | AviTag-6XHis | | | | |
| B-M3D4 | ectodomain Fusion | RBD*²-Linker- | 99 | kDa | Mammalian | 5 |
| | protein | AviTag-Linker- | | | Expi293 | |
| | | TEV-Linker- | | | cells | |
| | | ACE2*³-6XHis | | | | |

### Detection of Neutralizing Antibodies

Enzyme linked immunosorbent assay (ELISA). 96-well plates (Corning) were coated overnight at 4° C. with 300 ng/mL Neutravidin (Sigma) and blocked using 2.5% BSA (Omnipure) in PBST (phosphate buffer solution with 0.05% tween20) for 1 h at room temperature. After washes with PBST, 10 nM biotinylated SARS-CoV-2 RBD (B-M5B) and biotinylated SARS-CoV-2 RBD-ACE2 fusion proteins (B-3A and B-M3D4) were added to the plates and incubated at room temperature for 1 h. The unbound proteins were removed and washed 3 times by PBST. Different types of antibodies listed in Table 3 were added to each well and incubated at room temperature for 1 h. Then the antibodies were removed and washed 3 times by PBST, HRP-conjugated anti-mouse or anti-human IgG Fab antibody (Sigma) was added at the dilution of 1:10,000 in PBST containing 2.5% BSA and incubated at room temperature for 1 h. After washing with PBST three times SIGMAFAST™ OPD (o-Phenylenediamine dihydrochloride) substrate solution (Sigma) was added to the microplate and incubated at room temperature for 10-20 min. The OD450 absorbance was detected by SpectraMax iD3 microplate reader (Molecular Devices). The results were analyzed by GraphPad Prism 9.0.

Table 3 provides a list of antibodies used in the following experiments. These antibodies include neutralizing and non-neutralizing antibodies, allowing validation of the methods disclosed herein.

TABLE 3

| | | | Conc. | |
|---|---|---|---|---|
| | Source | Description | (ng/ml) | Reference |
| RBD1106 | HyTest | SARS-CoV-2 Spike RBD | 300 | |
| | | Antibody, mouse IgG | | |
| RBD5305 | | | 30 | |
| RBD5308 | | SARS-CoV-2 Spike RBD | | |
| | | Antibody, human chimeric MAbs | | |
| R107* | | SARS-CoV-2 Spike RBD | 300 | |
| | | Antibody, mouse IgG | | |
| E6* | Dr. Guang Yang | SARS-CoV-2 Spike RBD | 30 | Qiang M. |
| | Shanghei Tech | Antibody, human IgG | | et al. |
| | | | | (2021) |
| SIN- | AcroBiosystems | Anti-SARS-CoV-2 Spike RBD | 30 | |
| M130 | | Antibody, human chimeric mAb | | |
| | | (IgG1) | | |
| A02038 | Genscript | SARS-CoV-2 Spike S1 Antibody | 30 | |
| | | (HC2001), human chimeric | | |
| srbd- | InVivoGen | SARS-CoV-2 Spike RBD | 30 | Yuan M. |
| mab1 | | Antibody, human IgG1 (CR3022) | | et al. |
| | | | | (2020) |
| 91349 | ActiveMotif | Monoclonal antibody against | 30 | Qiang M. |
| | | SARS-CoV2 - Clone AM002414 | | et al. |
| 91383* | | Monoclonal antibody against | 30 | (2021) |
| | | SARS-CoV2 - Clone AM005505 | | |
| 91377 | | Monoclonal antibody against | 30 | |
| | | SARS-CoV2 - Clone AM015553 | | |
| 91361* | | Monoclonal antibody against | 30 | |
| | | SARS-CoV2 - Clone AM001414 | | |

*Neutralizing antibodies

### Detection of Neutralizing Antibodies for SARS-CoV-2

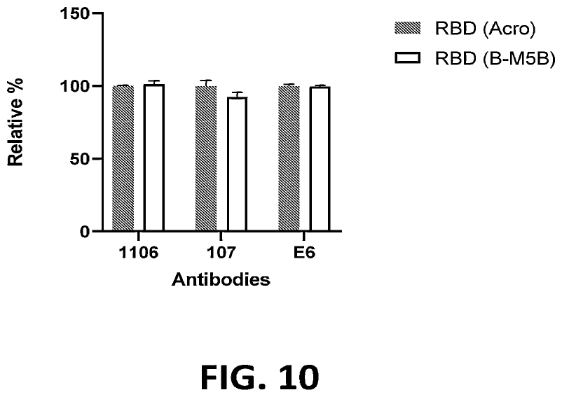

FIG. 10. B-M5B (Biotinylated SARS-CoV-2 RBD). B-M5B was compared to commercial biotinylated SARS-CoV-2 RBD protein (AcroBiosystem) using ELISA and there was no significant difference between commercial and in-house RBD proteins (P<0.05) (FIG. 10). Binding of each of three different antibodies (non-neutralizing antibody 1106, neutralizing antibody 107, and neutralizing antibody E6) is provided for each protein, with the data normalized to RBD (Acro). Because there is no significant different in antibody between RBD (Acro) and RBD (B-M5B), these results demonstrate that the viral protein used in the present experiments produce reliable results with respect to the detection of neutralizing antibodies.

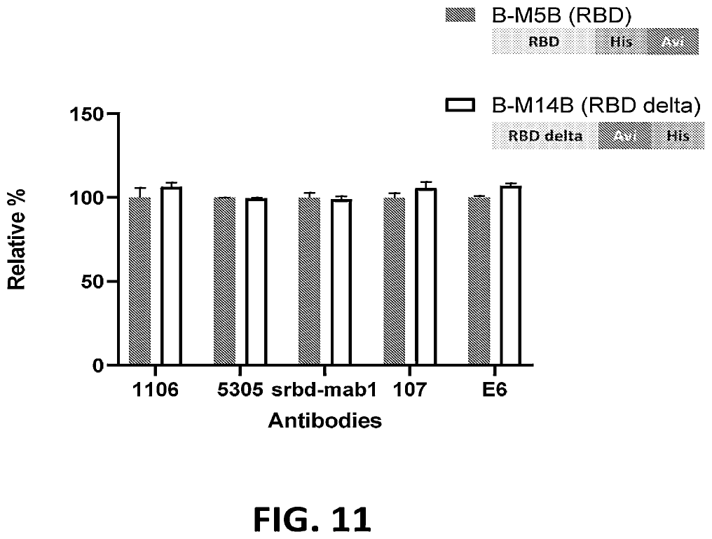

FIG. 11. B-M14B (Biotinylated SARS-CoV-2 RBD delta). As SARS-CoV-2 Delta variant is currently the predominant variant of the virus in the United States. The Delta variant cause more infectious and spreads faster than early forms of SARS-CoV-2. Biotinylated SARS-CoV-2 RBD delta (L452R, T478K) (B-M14B) was compared to early forms of SARS-CoV-2 (B-M5B) using ELISA and there was no significant difference (P<0.05) (FIG. 11). Five different antibodies were tested (1106, 5305, srbd-mab1, 107 and E6), and binding data was normalized to B-M5B (original SARS-CoV-2 RBD). In line with previously reported results, these results demonstrate no significant difference between the ability of neutralizing antibodies to bind to original SARS-CoV-2 strain and the delta variant. As new variants of high concern arise, such as, for example the omicron variant, they will be rapidly incorporated into compositions, methods and devices disclosed herein.

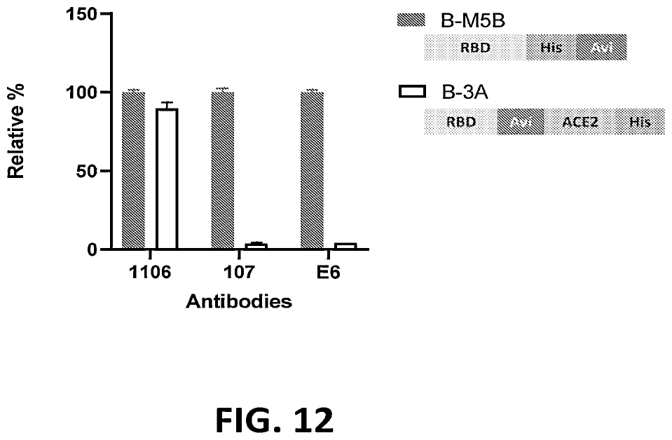

FIG. 12. B-3A (Biotinylated SARS-CoV-2 RBD and ACE2 fusion protein). Biotinylated SARS-CoV-2 RBD-ACE2 fusion protein (B-3A) was compared to SARS-CoV-2 RBD (B-M5B) using ELISA. Three different antibodies were tested, including one non-neutralizing antibody (1106) and two neutralizing antibodies (107 and E6). Data were normalized to B-M5B. There was no significant binding difference of non-neutralizing antibody 1106 (P<0.05) between B-M5B and M-3A but ˜92% reduced binding was observed for two different neutralizing antibodies (107 and E6) on B-3A fusion protein (FIG. 12). The difference between B-M5B and B-3A can be interpreted as the neutralizing antibody titer.

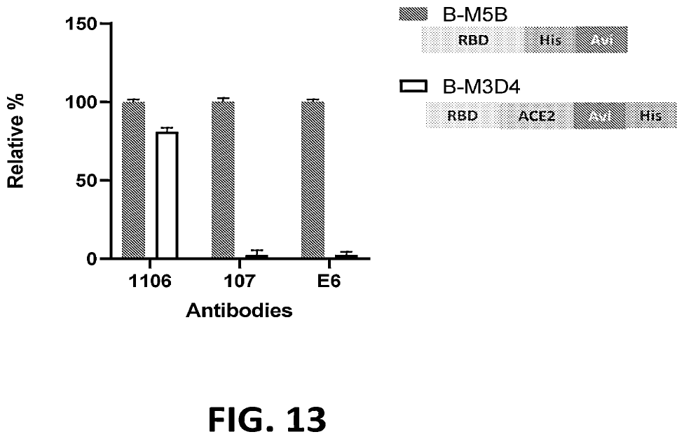

FIG. 13. B-M3D4 (Biotinylated SARS-CoV-2 RBD and truncated ACE2 fusion protein with cysteine mutations). To further stabilize the fusion protein in a conformation in which RBM stays bound to ACE2, a fusion (stapled) protein B-M3D4 was constructed. Biotinylated SARS-CoV-2 RBD-ACE2 fusion protein (B-M3D4) was compared to SARS-CoV-2 RBD (B-M5B) using ELISA. Data was again normalized to B-M5B. A ˜98% reduced binding was observed for two different neutralizing antibodies (107 and E6) on B-M3D4 fusion protein while binding of non-neutralizing antibody (1106) was minimally reduced (less than 20%) (FIG. 13). The difference between B-M5B and B-M3D4 can be thus interpreted as the neutralizing antibody titer.

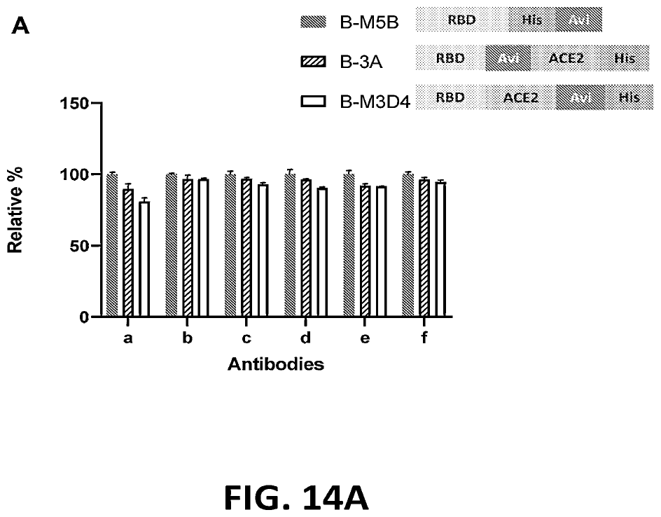

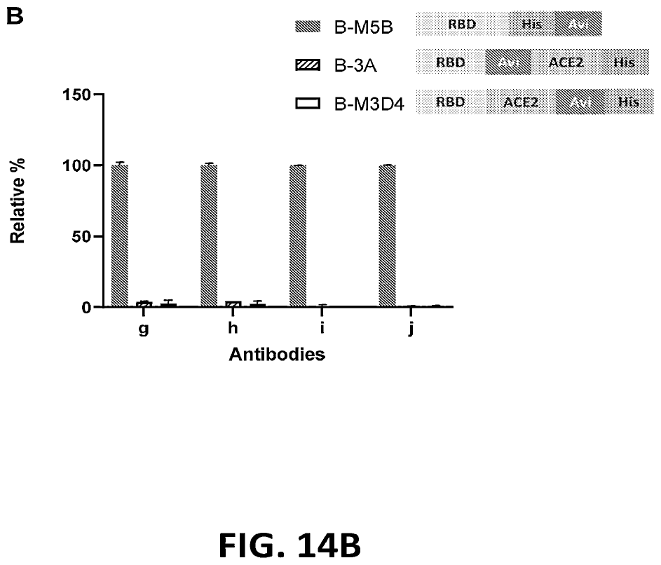

FIGS. 14A-14B. Probing with panel of non-neutralizing and neutralizing antibodies directed against RBD. For further investigation of binding anti-SARS-CoV-2 RBD antibodies, a total 6 non-neutralizing antibodies were compared (FIG. 12A). Antibodies tested were as follows: a: 1106, b: 5305, c: 5308, d: SIN-M130, e: A02038, f: 91349. The binding of each of these antibodies was tested against SARS-Cov-2 RBD (B-M5B), fusion protein (B-3A), and stapled fusion protein (B-M3D4). There was a variation of 2-20% reduced binding with non-neutralizing antibodies, dependent on antibody (FIG. 14A). Thus, these results demonstrate minimal difference between each protein construct with respect to non-neutralizing antibody binding.

Next, four neutralizing antibodies were tested with B-5 MB. B-3A, and B-M3D4. Each of these antibodies directly competes with ACE2 for binding. Each neutralizing antibodies showed 95-99% reduced binding on both fusion proteins B-3A and B-M3D4 (FIG. 14B). Antibodies tested were as follows: g: 107, h: E6, i: 91383, j: 91361. These results demonstrate that modified viral proteins of the present disclosure effectively prohibit binding of neutralizing antibodies.

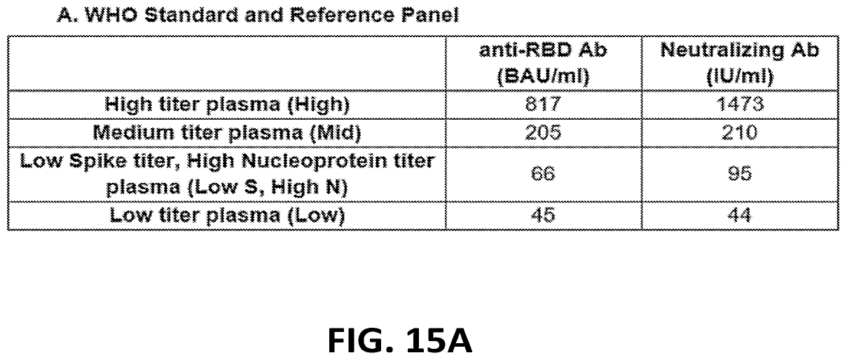

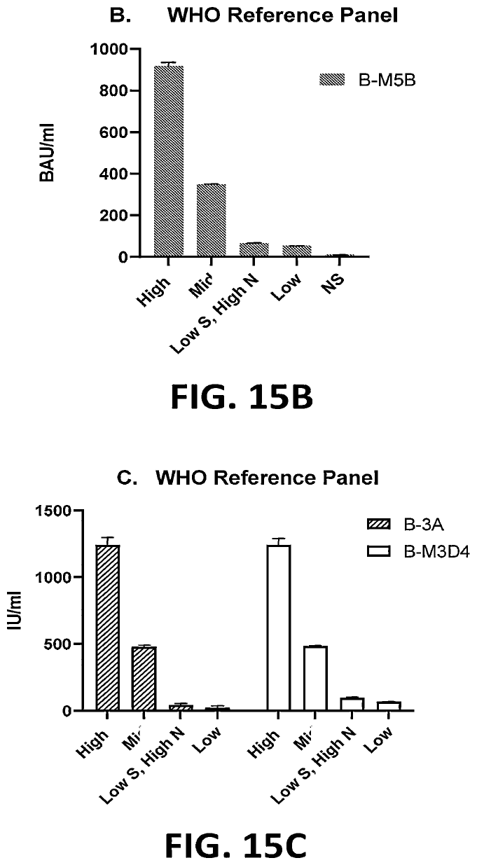

FIGS. 15A-15C. WHO Standard and Reference Panel (NIBSC). The WHO Standard for anti-SARS-CoV-2 immunoglobulin is established for the calibration and harmonization of serological assays detecting anti-SARS-CoV-2 neutralizing antibodies, which is pooled plasma obtained from eleven individuals recovered from SARS-CoV-2 infection. The assigned potency of the WHO standard for SARS-CoV-2 neutralizing antibody activity is 1000 IU/ml. For binding antibody assays, an arbitrary unitage of 1000 BAU/ml can be used to assist the comparison of assays detecting anti-RBD IgG. The WHO Reference Panel of anti-SARS-CoV-2 immunoglobulin consists of the four pooled plasma samples obtained from individuals recovered from COVID-19 and one negative control plasma obtained from healthy blood donors before 2019. Anti-SARS-CoV-2 antibody titers of each panel were calculated as geometric mean of the potencies obtained from the collaborative study based on WHO Standard plasma (FIG. 15A).

To evaluate the RBD/ACE2 fusion proteins. WHO Standard and Reference Panel plasma were tested with biotinylated SARS-CoV-2 RBD (B-M5B) and RBD-ACE2 fusion proteins (B-3A and B-M3D4) using ELISA. The binding antibody unit (BAU/ml) to in-house SARS-CoV-2 RBD (B-M5B) of each WHO panel plasma was calculated based on WHO Standard binding intensity assigned as 1000 BAU/ml (FIG. 15B). The neutralizing antibody titer (IU/ml) was obtained from the difference intensity between RBD (B-M5B) and RBD/ACE2 fusion protein (B-3A or B-M3D4) (FIG. 15C).

The results with RBD/ACE2 fusion proteins (FIGS. 15B and 15C) were comparable to WHO reference Panel Data (FIG. 15A), demonstrating that RBD/ACE2 fusion proteins (B-3A and B-M3D4) can be used in the detection and quantification of neutralizing anti-SARS-CoV-2 antibodies. Thus, the foregoing examples demonstrate that the methods and compositions disclosed herein are effective at detecting and quantifying neutralizing antibodies against a pathogen in a sample. Moreover, the covalent coupling between the viral protein and the receptor protein (through a linker and/or a covalent bond at the interface) reduce or prevent dissociation of the two viral protein/receptor complex.

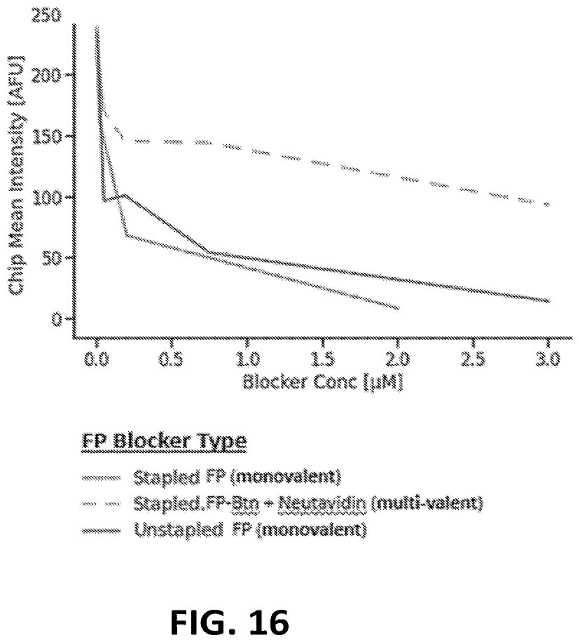

FIG. 16 demonstrates that the sensitivity of the assay for neutralizing antibodies depends on the structure of the modified viral protein used to selectively bind non-neutralizing antibodies (e.g., stapled and/or fusion proteins or multivalent complexes thereof), also referred to as “FP blockers”. The Y axis provides the mean chip intensity (AFU) for the detection of neutralizing antibodies after treatment of a sample with one of the following modified viral proteins (“FP blockers”): (1) monomeric stapled RBD/ACE2 fusion protein (solid light gray line); (2) a multivalent complex comprising biotinylated stapled RBD/ACE2 fusion proteins bound to neutravidin (dashed grey line); and (3) monomeric unstapled RBD/ACE2 fusion protein (solid dark gray line). The X axis provides the concentration of the modified protein. The data provided in FIGS. 16-20 was obtained following a method analogous to that set forth in FIG. 7. As shown in FIG. 16, the multimeric fusion protein complex provided the best results (highest neutralizing antibody signal) across a broad range of FP blocker concentrations.

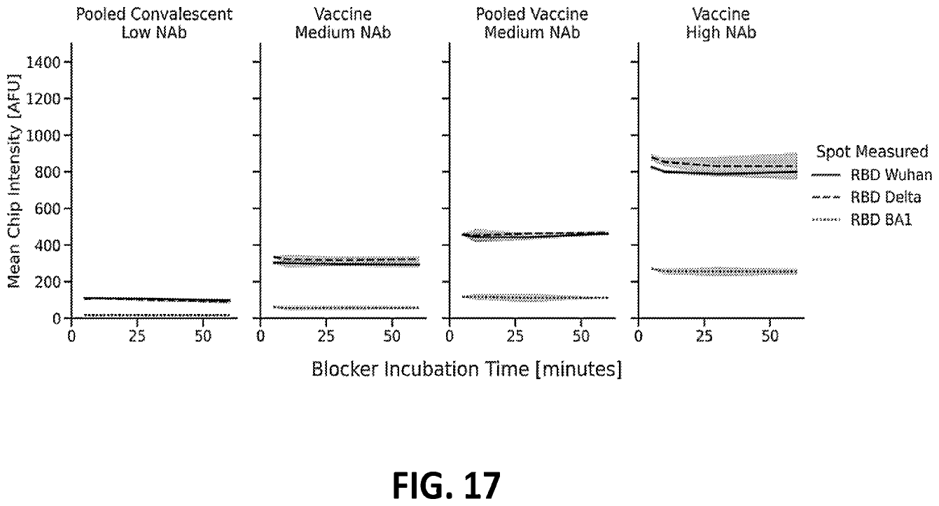

FIG. 17 provides neutralizing antibody data from four different samples binned according to neutralizing antibody titers ascertained by a live virus neutralization assay: (1) a pooled sample of convalescent plasma having low neutralizing antibody titers (left). (2) a sample from a vaccinated individual having a medium neutralizing antibody titer (center left). (3) a pooled sample from vaccinated people having a medium neutralizing antibody titer (center right), and (4) a vaccinated individual with a high neutralizing antibody titer (right). Neutralizing antibodies were measured for three different SARS-CoV2 RBD variants: (1) the original Wuhan variant (solid black line); (2) the delta variant (dashed dark grey line); and (3) the Omicron BA. I subvariant (dotted grey line). Infected individuals were infected prior to the emergence of the Omicron variant. The Y axes provide the intensity of the neutralizing antibody detection (AFU), and the X axes provide the blocker incubation time (multimeric FP blocker).

Several trends are evident from the data in FIG. 17. First, the neutralizing antibody signal was stable across all incubation times, indicating rapid sequestering of non-neutralizing antibodies within ˜5 minutes. Second, signal degradation does not occur over time, demonstrating the stability of the antibody-protein complex comprising neutralizing antibodies. Third, neutralizing antibody levels for the Omicron BA. I subvariant were low in all samples, except for the vaccinated individual (right), who was likely to have been infected with the omicron variant after vaccination. The low levels of Omicron-specific neutralizing antibodies are in contrast to the neutralizing antibodies directed at RBD Wuhan and RBD Delta. Thus, the assay described herein can differentiate between variant-specific immunity profiles across individuals and/or populations.

Methods of quantifying neutralizing antibodies using the Q-NAb Test. Some aspects of the present disclosure are utilized in the “Q-NAb Test” for measuring neutralizing antibodies against one or more viral proteins. The Q-NAb Test is processed on a microfluidic centrifugal disc using a cloud-based, field-use instrument, the Autolab-20. The heart of the Q-NAb is a nitrocellulose microarray, on which a viral surface protein (e.g., the receptor-binding-domain (RBD) proteins-currently the spike protein or a portion thereof derived from the Wuhan, Delta, and Omicron variants of SARS-CoV2) are printed. The assay directly detects NAbs binding to RBD proteins on the microarray by first depleting non-neutralizing anti-RBD antibodies (NNAb) from the sample using a novel ACE2-RBD fusion protein (FP), using methods of the present disclosure. As described throughout the present disclosure, this FP is constructed by fusing the human ACE2 receptor—the initial target for viral entry—to RBD, effectively concealing the receptor binding motif (RBM) epitopes where NAbs bind. The Q-NAb Test is available in both benchtop (1 hour) and centrifugal disc formats (15 minutes). The benchtop Q-NAb was used to evaluate 219 clinical samples, including 32 negative. 33 convalescent. 135 vaccine, and 19 mixed (vaccine+infection) samples. Q-NAb results and MN50 values were correlated using both parametric fit (4-parameter logistic (4PL) regression model) and non-parametric correlations (Spearman's rank). A set of 80 longitudinal vaccine samples was used to build an exponential decay model to predict NAb levels post-vaccination and estimate NAb half-life.

As described in detail below, the Q-NAb test is a qualitative test for measuring live virus neutralization titers, and the Q-NAb Test had a sensitivity of 96.5% (95% CI: 92.0%-98.9%) and specificity of 100.0% (95% CI: 91.8%-100.0%). As a quantitative test. Q-NAb signal was highly correlated with log MN50 (4PL. R2=0.85, p<0.05). In pre-2021 convalescent samples, we observed an average 77.7% reduction in NAb activity against Omicron BA. 1 and an 81.2% reduction against Omicron BA.2. Using longitudinal samples from mRNA-1273 vaccinated individuals, an exponential regression model to predict NAb decay over time (R2=0.84, p<0.05) was developed. NAb half-life was estimated at 89.4 days (95% CI, 75.4 to 109.7 days).

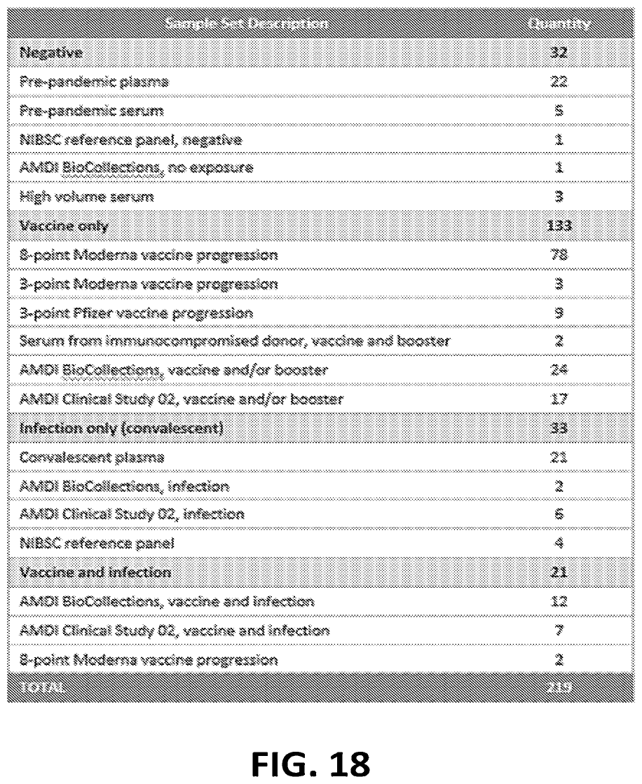

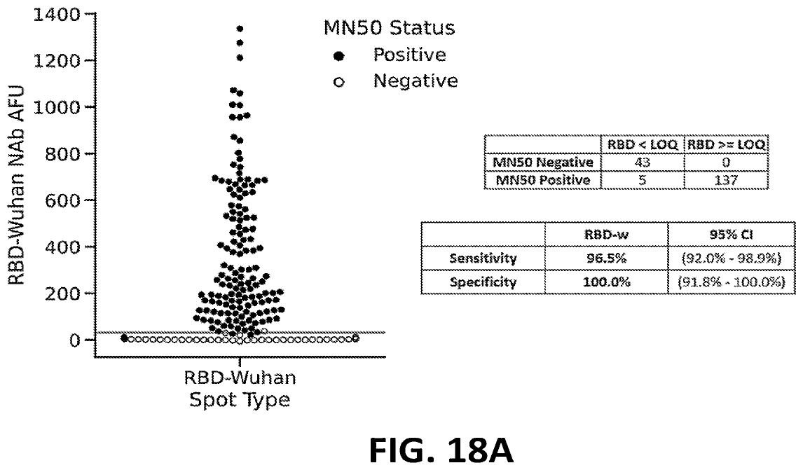

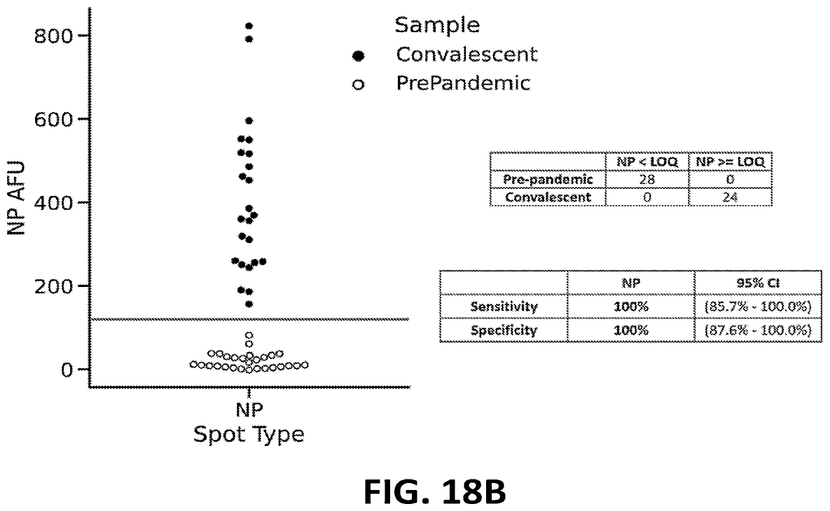

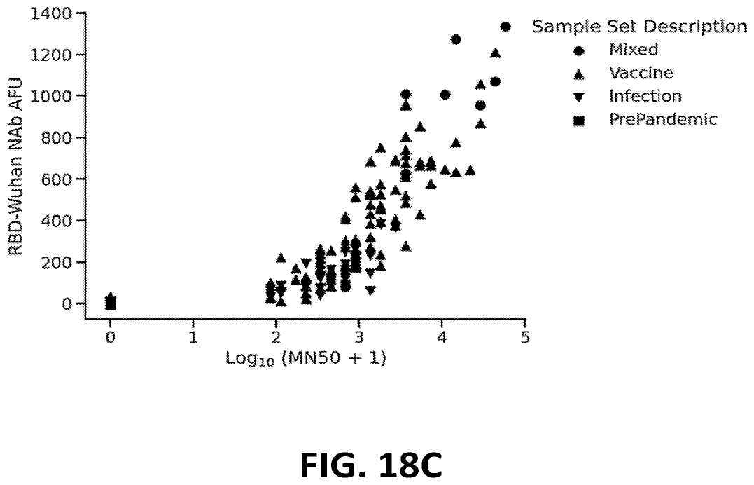

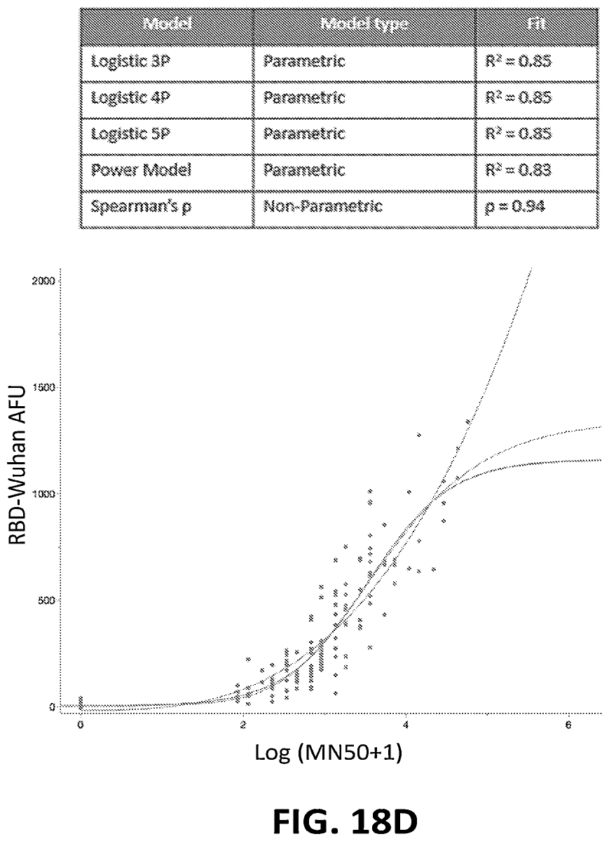

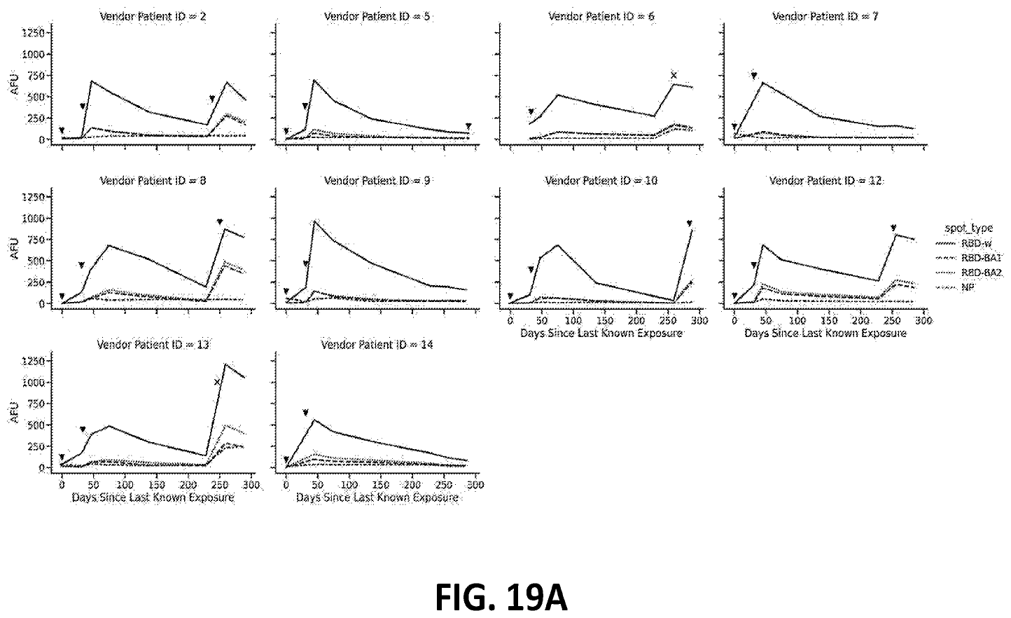

FIG. 18 provides a table describing the sampled used in the analysis set forth in FIGS. 18A-20 The samples tested each fall into one of several categories: (1) samples from vaccinated individuals (133 samples), including multi-point samples (samples taken from the same vaccinated subject at different time post-vaccination) and single-point samples from a variety of individuals, including clinical trial volunteers and immunocompromised patients; (2) samples from unvaccinated individuals with natural infection (33 samples), including convalescent plasma samples; (3) samples from vaccinated individuals with natural infection (21 samples), and (4) pre-pandemic or negative samples (32 samples) including plasma and serum. Each sample had been separately tested using the microneutralization assay (e.g., the assay set forth in F. Amanat et al., An In Vitro Microneutralization Assay for SARS-CoV-2 Serology and Drug Screening. 58 Curr Protoc. Microbiol. E108 (2020), or a variant on that assay).

FIG. 18A provides a plot that includes the samples described in the table of FIG. 18. The Y axis represents the quantitative signal for neutralizing antibodies against the RBD Wuhan (AFU). Samples having a positive MN₅₀ status (a detectable neutralizing antibody titer for antibodies directed at SARS-CoV2) are represented by solid black dots. Samples having a negative MN₅₀ status (no detectable neutralizing antibody titer for antibodies directed at SARS-CoV2) are represented by unfilled dots. As shown in FIG. 18A, for nearly all MN₅₀ negative samples (unfilled dots), no neutralizing antibodies were detected using a method of the present disclosure. In contrast, for nearly all MN₅₀ positive samples (sold black dots), neutralizing antibodies were detected using the same method of the present disclosure. No outliers among the 43 MN50 negative samples were observed, exhibiting a specificity of 100%. Only five outliers among the 142 MN50 positive samples were observed, which exhibited a low level of neutralizing antibodies marginally below the signal threshold. Thus. FIG. 18A demonstrates that this method of the present disclosure works with a sensitivity of 96.5% for RBD Wuhan and a selectivity of 100% for the same antigen.

FIG. 18B provides a plot that includes the samples described in the table of FIG. 18. The Y axis represents the quantitative signal for neutralizing antibodies against the nucleoprotein (“NP”) from the original Wuhan variant of SARS-CoV2 (AFU). Samples derived from convalescent plasma taken from individuals previously infected with SARS-CoV2 are represented by solid black dots. Samples taken from individuals prior to the pandemic (no prior known infection by SARS-CoV2) are represented by unfilled dots. As shown in FIG. 18A, for all pre-pandemic samples (unfilled dots), no neutralizing antibodies directed at SARS-CoV2 NP were detected using a method of the present disclosure. In contrast, for all samples derived from convalescent plasma (sold black dots), neutralizing antibodies directed at SARS-CoV2 NP were detected using the same method. Thus, FIG. 18B demonstrates that this method of the present disclosure worked with 100% sensitivity and 100% selectivity for SARS-CoV2 NP in this sample set.

FIGS. 18C and 18D demonstrate a clear correlation between the NAb detection using a method of the present disclosure and the MN50 values derived from the prior art microneutralization assay. All values were derived from samples identified in FIG. 18. Pre-pandemic samples (“PrePandemic”) are each represented by a square. Samples derived from vaccinated individuals (“Vaccine”) are each represented by a triangle with the point upwards. Samples derived from previously infected individuals (e.g., convalescent plasma) (“Infection”) are each represented by a triangle with the point downwards. Samples derived from individuals who have been both vaccinated and infected (“Mixed”) are each represented by a circle. The Y axis represents the quantitative signal for neutralizing antibodies against the RBD Wuhan (AFU) determined by a method of the present disclosure on a linear scale. The X axis corresponds to the MN50 values of the samples obtained using the microneutralization assay of the prior art on a logarithmic scale (values that correspond to the NAb titer).

As shown in FIG. 18C, all pre-pandemic samples did not generate any appreciable NAb signal using either method. Further, all other sample types (Mixed, Vaccine, and Infected) fall along an exponential line, demonstrating a close correlation between the two methods. Samples with relatively low NAb signal (roughly 100-300 AFU using a method of the present disclosure and a Log of 2-3 MN50 value) are tightly correlated, as are samples with relatively high NAb signal (roughly 600-1400 AFU using a method of the present disclosure and a Log of 4-5 MN50 value). This correlation is depicted in FIG. 18D, which shows a regression analysis demonstrating a close correlation between the two methods using both parametric fits (0.83<R²<0.85) and non-parametric correlations (Spearman's ρ: 0.94). Thus, FIGS. 18C and 18D demonstrate that the NAb values quantified by methods of the present disclosure closely correlate with MN50 values determined by the microneutralization assay and that values derived from methods of the present disclosure can be correlated to MN50 values in a predictable and consistent manner. As such, methods of the present disclosure can be used in place of the costly and slow microneutralization assay to obtain a qualitative and/or quantitative NAb titer.

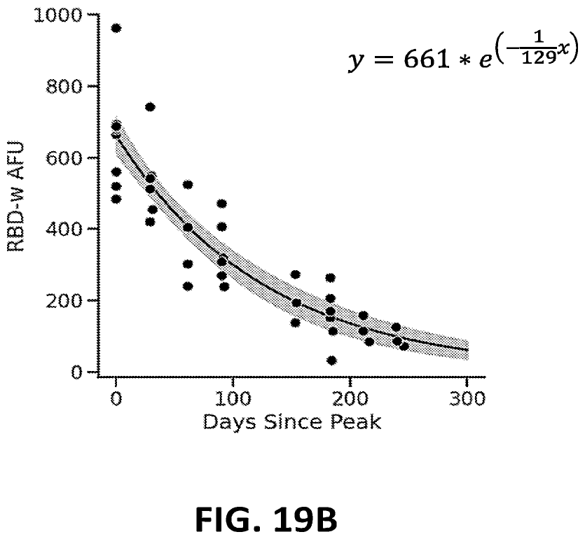

FIG. 19A provides an analysis of NAb levels of ten individuals over a period of almost 300 days after vaccination with an mRNA vaccine. Four different NAb populations were analyzed, specifically, those directed at the following antigens: (1) RBD Wuhan (solid line); (2) RBD for the BA.1 subvariant (dashed line); (3) RBD for the BA.2 subvariant (dotted line); and (4) nucleoprotein (alternating dash-dot line). Time points where the individual received a dose of a SARS-CoV2 vaccine are represented by triangles. Time points where the individual was infected with SARS-CoV2 are marked by an X.

As seen in FIG. 19, the immunity profile of different individuals can be discerned using methods of the present disclosure. For example, patient ID 7 (top right) was vaccinated with two doses of an mRNA vaccine, thereby increasing their NAb titer against the RBD Wuhan, but without materially increasing their NAb titer against RBD BA. 1. RBD BA.2, or nucleoprotein (“NP”). This is consistent with the fact that the vaccine administered utilized the Wuhan variant's spike protein to generate the patient's immunity. Further, the RBD Wuhan NAb titer for Patient ID 7 decreased over time, consistent with the waning immunity that has been characterized in mRNA vaccines against SARS-CoV2. Patient ID 5, Patient ID 9, and Patient ID 14 each exhibited a similar profile.

Patient ID 2 (top left) exhibited a similar NAb profile following their first two doses of vaccine (high RBD Wuhan NAb titer with a gradual decrease over time, with little increase in NAbs against other antigens tested). However. Patient ID 2 received a third dose (i.e., a booster dose), which increased not only the NAb titer against RBD Wuhan, but also RBD BA.1 and RBD BA.2, consistent with clinical observations to date. The NAb titer against NP did not increase for Patient ID 2 following any of the vaccine doses, consistent with the fact that the mRNA vaccines do not contain or encode for SARS CoV2 nucleoprotein. Patient ID 8. Patient ID 10, and Patient ID 12 each exhibited a similar profile.

Patent ID 13 (bottom left) received two vaccine doses and was subsequently infected with SARS-CoV2 (potentially an Omicron variant). Following infection, the NAb titer increased for antibodies directed at RBD Wuhan. RBD BA.1 and RBD BA.2. However, in contrast to vaccination, infection leads to a substantial increase in NAb titer against NP. This is consistent with the fact that NP is present in the SARS-CoV2 virus and antigenic. Patient ID 6 exhibited a similar profile.

Thus, as shown in FIG. 19A, in some embodiments, methods of the present disclosure can assist in the analysis of an individual's NAb profile and distinguish between natural infection and vaccination (compare Patient ID 8 to Patient ID 13). Further, in some embodiments, methods of the present disclosure can assist in the analysis of an individual's NAb profile and distinguish between infection with different variants and/or subvariants of a particular virus.

FIG. 19B shows the decrease of NAb levels in individuals over time for samples (including those derived from individuals analyzed in FIG. 19A). The Y axis represents the quantitative signal for neutralizing antibodies against the RBD Wuhan (AFU). The X axis represents the days since peak NAb levels. The extrapolated line (solid line) shows that gradual decrease in NAb levels largely follows an exponential decay pattern. From this regression analysis, a decay equation has been derived, also shown in FIG. 19B. From this, a half-life for NAbs against RBD Wuhan has been calculated to be 89.5 day (95% CI, 75.4 to 109.7), which is consistent with literature reports (see. e.g., Makowski NEJM 2021 384; 23). Moreover, the following equation has been derived that provides the number of days until an individual will reach a critical NAb threshold value:

$$Days until Critical NAb = 129 * ln \frac{NAb AFU}{{φ}_{Critical}}$$

where NAb AFU is the NAb level measured by the Q-NAb test and ϕ_{critical} (measured in AFU) is a level below which NAb levels are considered critical.

Thus, in some embodiments, an individual's NAb AFU can be obtained using a method of the present disclosure, plugged into the above equation and a predicted time point is provided by the equation (i.e., a calculated number of days until the individual's NAb titer drops below a critical value). In some embodiments, the predicted time point could be used to schedule a booster dose, thereby ensuring that the individual receives additional protection only when necessary based on their specific immunity profile. φ_{critical} can be set, for example, based on public health advisories from governmental or scientific organizations (e.g., NIAID, WHO, NIH, etc.). In some embodiments, φ_{critical} is calculated from a threshold value derived from an MN50 value, for example, one set by one of these agencies. Thus, in this manner, in some embodiments, the present disclosure provides a method of vaccinating an individual that includes (i) calculating a time point when the individual will have an NAb level below a threshold level and (ii) administering a dose of vaccine to the individual).

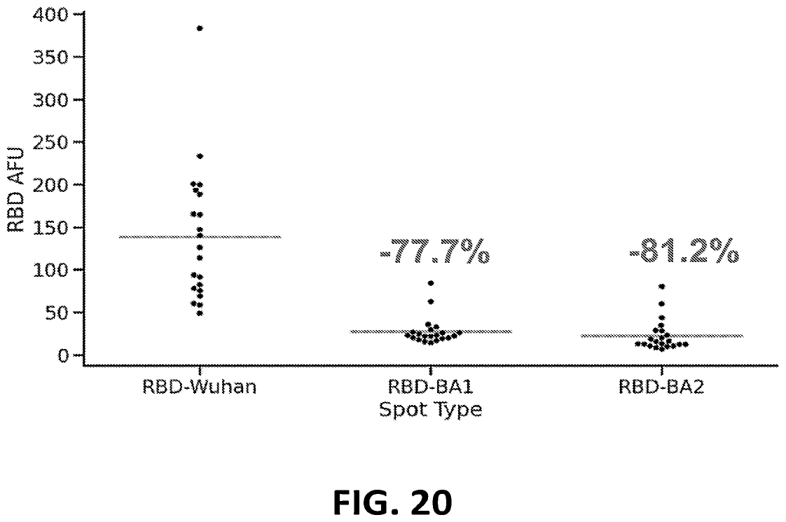

FIG. 20 depicts the difference in NAb titers against three different variants of the SARS-CoV2 spike protein (RBD-Wuhan, RBD-BA. 1, and RBD-BA.2) present in samples derived from the same set of vaccinated individuals. As shown in FIG. 20, the average NAb levels for BA.1 and BA.2 subvariants were 78% and 81% lower, respectively, than the average NAb level for antibodies directed at RBD Wuhan. Thus, FIG. 20 shows that methods of the present disclosure can be used to assess the likely threat of a newly characterized variant of a virus (e.g., the BA.1 and BA.2 Omicron subvariants) against a vaccinated population by assessing the extent to which the new variant(s) evade immunity provided by existing neutralizing antibodies in an individual or population. In this instance, the method employed predicted a significant level of immune escape by the BA.1 and BA.2 Omicron subvariants, which has been observed at the population scale.

As demonstrated by the foregoing, the Q-NAb Test is a cell-free, rapid assay that measures levels of neutralization activity against past and current SC2 variants and can be used in both laboratory and field settings. Q-NAb results are highly predictive of live virus neutralization, strongly correlating with live virus neutralization (MN50) values. An exponential regression model predicted NAb decay over time and estimated a post-vaccination NAb half-life of 89.4 days. The Q-NAb Test can supplement live virus neutralization testing to rapidly measure new vaccine efficacy and facilitate population surveillance of NAb levels against new SC2 variants. The Q-NAb test is a rapid, cell-free NAb test that highly correlates with MNA. The Q-NAb Test is a 15-minute microfluidic assay that quantitatively measures IgG-specific NAb levels against the Wuhan strain of SC2, and qualitatively assesses NAb levels against other SC2 variants (e.g., Delta and Omicron BA.1, BA.2). Using the Q-NAb Test, an exponential decay model was developed to predict NAb activity and half-life post-vaccination. As described herein, this exponential model that can be used to guide timing of vaccine dose administration and/or the administration of anti-viral drugs to a patient.

While preferred embodiments of the present disclosure have been shown and described herein, it will be obvious to those skilled in the art that such embodiments are provided by way of example only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein can be employed in practicing the present disclosure. It is therefore contemplated that the present disclosure shall also cover any such alternatives, modifications, variations or equivalents. It is intended that the following claims define the scope of the present disclosure and that methods and structures within the scope of these claims and their equivalents be covered thereby.

Provided below are the amino acid sequences referred to in the present disclosure:

```
SEQ ID NO: 1
                10 20 30 40 50
MFVFLVLLPL VSSQCVNLTT RTQLPPAYTN SFTRGVYYPD KVERSSVLHS
                60 70 80 90 100
TQDLFLPFFS NVTWFHAIHV SGTNGTKRED NPVLPENDGV YFASTEKSNI
              110 120 130 140 150
IRGWIFGTTL DSKTQSLLIV NNATNVVIKV CEFQFCNDPF LGVYYHKNNK
              160 170 180 190 200
SWMESEFRVY SSANNCTFEY VSQPFLMDLE GKQGNEKNLR EFVEKNIDGY
              210 220 230 240 250
FKIYSKHTPI NLVRDLPQGF SALEPLVDLP IGINITRFQT LLALHRSYLT
              260 270 280 290 300
PGDSSSGWTA GAAAYYVGYL QPRTELLKYN ENGTITDAVD CALDPLSETK
              310 320 330 340 350
CTLKSFTVEK GIYQTSNERV QPTESIVREP NITNLCPFGE VENATRFASV
              360 370 380 390 400
YAWNRKRISN CVADYSVLYN SASFSTFKCY GVSPTKLNDL CFTNVYADSF
              410 420 430 440 450
VIRGDEVRQI APGQTGKIAD YNYKLPDDFT GCVIAWNSNN LDSKVGGNYN
              460 470 480 490 500
YLYRLFRKSN LKPFERDIST EIYQAGSTPC NGVEGENCYF PLQSYGFQPT
              510 520 530 540 550
NGVGYQPYRV VVLSFELLHA PATVCGPKKS TNLVKNKCVN FNENGLTGTG
              560 570 580 590 600
VLTESNKKEL PFQQFGRDIA DTTDAVRDPQ TLEILDITPC SFGGVSVITP
              610 620 630 640 650
GTNTSNQVAV LYQDVNCTEV PVAIHADQLT PTWRVYSTGS NVFQTRAGCL
              660 670 680 690 700
IGAEHVNNSY ECDIPIGAGI CASYQTQTNS PRRARSVASQ SIIAYTMSLG
              710 720 730 740 750
AENSVAYSNN SIAIPTNFTI SVTTEILPVS MTKTSVDCTM YICGDSTECS
              760 770 780 790 800
NLLLQYGSFC TQLNRALTGI AVEQDKNTQE VFAQVKQIYK TPPIKDFGGF
              810 820 830 840 850
NFSQILPDPS KPSKRSFIED LLENKVTLAD AGFIKQYGDC LGDIAARDLI
              860 870 880 890 900
CAQKENGLTV LPPLLTDEMI AQYTSALLAG TITSGWTFGA GAALQIPFAM
              910 920 930 940 950
QMAYRENGIG VTQNVLYENQ KLIANQFNSA IGKIQDSLSS TASALGKLQD
              960 970 980 990 1000
VVNQNAQALN TLVKQLSSNF GAISSVLNDI LSRLDKVEAE VQIDRLITGR
            1010 1020 1030 1040 1050
LQSLQTYVTQ QLIRAAEIRA SANLAATKMS ECVLGQSKRV DFCGKGYHLM
            1060 1070 1080 1090 1100
SFPQSAPHGV VFLHVTYVPA QEKNFTTAPA ICHDGKAHFP REGVFVSNGT
            1110 1120 1130 1140 1150
HWFVTQRNFY EPQIITTDNT FVSGNCDVVI GIVNNTVYDP LQPELDSEKE
            1160 1170 1180 1190 1200
ELDKYFKNHT SPDVDLGDIS GINASVVNIQ KEIDRLNEVA KNLNESLIDL
            1210 1220 1230 1240 1250
QELGKYEQYI KWPWYIWLGF IAGLIAIVMV TIMLCCMTSC CSCLKGCCSC
            1260 1270
GSCCKFDEDD SEPVLKGVKL HYT
SEQ ID NO: 2
                10 20 30 40 50
MSSSSWLLLS LVAVTAAQST IEEQAKTELD KFNHEAEDLF YQSSLASWNY
                60 70 90 100
NTNITEENVQ NMNNAGDKWS AFLKEQSTLA QMYPLQEIQN LTVKLQLQAL
              110 120 130 140 150
QQNGSSVLSE DKSKRINTIL NTMSTIYSTG KVCNPDNPQE CLLLEPGLNE
              160 170 180 190 200
IMANSLDYNE RLWAWESWRS EVGKQLRPLY EEYVVLKNEM ARANHYEDYG
              210 220 230 240 250
DYWRGDYEVN GVDGYDYSRG QLIEDVEHTF EEIKPLYEHL HAYVRAKLMN
              260 270 280 290 300
AYPSYISPIG CLPAHLLGDM WGREWTNLYS LTVPFGQKPN IDVTDAMVDQ
              310 320 330 340 350
AWDAQRIFKE AEKFFVSVGL PNMTQGFWEN SMLTDPGNVQ KAVCHPTAWD
              360 370 380 390 400
LGKGDFRILM CTKVTMDDFL TAHHEMGHIQ YDMAYAAQPF LLRNGANEGF
              410 420 430 440 450
HEAVGEIMSL SAATPKHLKS IGLLSPDFQE DNETEINELL KQALTIVGTL
              460 470 480 490 500
PFTYMLEKWR WMVFKGEIPK DQWMKKWWEM KREIVGVVEP VPHDETYCDP
              510 520 530 540 550
ASLFHVSNDY SFIRYYTRTL YQFQFQEALC QAAKHEGPLH KCDISNSTEA
              560 570 580 590 600
GQKLENMLRL GKSEPWTLAL ENVVGAKNMN VRPLLNYFEP LFTWLKDQNK
              610 620 630 640 650
NSFVGWSTDW SPYADQSIKV RISLKSALGD KAYEWNDNEM YLFRSSVAYA
              660 670 680 690 700
MRQYFLKVKN QMILFGEEDV RVANLKPRIS FNFFVTAPKN VSDIIPRTEV
              710 720 730 740 750
EKAIRMSRSR INDAFRLNDN SLEFLGIQPT LGPPNQPPVS IWLIVFGVVM
              760 770 780 790 800
GVIVVGIVIL IFTGIRDRKK KNKARSGENP YASIDISKGE NNPGFQNTDD
VQTSF
SEQ ID NO: 3
                10 20 30 40 50
MLLVNQSHQG FNKEHTSKMV SAIVLYVLLA AAAHSAFAGG RVQPTESIVR
                60 70 80 90 100
FPNITNLCPF GEVENATRFA SVYAWNRKRI SNCVADYSVL YNSASFSTEK
              110 120 130 140 150
CYGVSPTKLN DLCFTNVYAD SFVIRGDEVR QIAPGQTGKI ADYNYKLPDD
              160 170 180 190 200
FTGCVIAWNS NNLDSKVGGN YNYLYRLFRK SNLKPFERDI STEIYQAGST
              210 220 230 240 250
PCNGVEGENC YFPLQSYGFQ PTNGVGYQPY RVVVLSFELL HAPATVCGPK
              260 270 280 290 300
KSTNLVKNKC VNFSGGGGSG GGGSSGGENL YFQGGSGGGG SGGGGSSQST
              310 320 330 340 350
IEEQAKTFLD KFNHEAEDLF YQSSLASWNY NTNITEENVQ NMNNAGDKWS
              360 370 380 390 400
AFLKEQSTLA QMYPLQEIQN LTVKLQLQAL QQNGSSVLSE DKSKRINTIL
              410 420 430 440 450
NTMSTIYSTG KVCNPDNPQE CLLLEPGLNE IMANSLDYNE RLWAWESWRS
              460 470 480 490 500
EVGKQLRPLY EEYVVLKNEM ARANHYEDYG DYWRGDYEVN GVDGYDYSRG
              510 520 530 540 550
QLIEDVEHTF EEIKPLYEHL HAYVRAKLMN AYPSYISPIG CLPAHLLGDM
              560 570 580 590 600
WGRFWTNLYS LTVPFGQKPN IDVTDAMVDQ AWDAQRIFKE AEKFFVSVGL
              610 620 630 640 650
PNMTQGFWEN SMLTDPGNVQ KAVCHPTAWD LGKGDERILM CTKVTMDDEL
              660 670 680 690 700
TAHHEMGHIQ YDMAYAAQPF LLRNGANEGF HEAVGEIMSL SAATPKHLKS
              710 720 730 740 750
IGLLSPDFQE DNETEINFLL KQALTIVGTL PFTYMLEKWR WMVFKGEIPK
              760 770 780 790 800
DQWMKKWWEM KREIVGVVEP VPHDETYCDP ASLFHVSNDY SFIRYYTRTL
              810 820 830 840 850
YQFQFQEALC QAAKHEGPLH KCDISNSTEA GQKLENMLRL GKSEPWTLAL
              860 870 880 890 900
ENVVGAKNMN VRPLLNYFEP LFTWLKDQNK NSFVGWSTDW SPYADQSIKV
              910 920 930 940 950
RISLKSALGD KAYEWNDNEM YLFRSSVAYA MRQYFLKVKN QMILFGEEDV
              960 970 980 990 1000
RVANLKPRIS FNFFVTAPKN VSDIIPRTEV EKAIRMSRSR INDAFRLNDN
            1010 1020 1030 1040
SLEFLGIQPT LGPPNQPPVS GGLNDIFEAQ KIEWHEGGHH HHHH
SEQ ID NO: 4
                10 20 30 40 50
MGWSCIILFL VATATGVHSR VQPTESIVRF PNITNLCPFG EVENATRFAS
                60 70 80 90 100
VYAWNRKRIS NCVADYSVLY NSASFSTFKC YGVSPTKLND LCFTNVYADS
              110 120 130 140 150
FVIRGDEVRQ IAPGQTGKIA DYNYKLPDDF TGCVIAWNSN NLDSKVGGNY
              160 170 180 190 200
NYLYRLERKS NLKPFERDIS TEIYQAGSTP CNGVEGENCY FPLQSYGFQP
              210 220 230 240 250
TNGVGYQPYR VVVLSFELLH APATVCGPKK STNLVKNKCV NEGGSGLNDI
              260 270 280 290 300
FEAQKIEWHE GGSENLYFQG GSGGGGSGGG GSSQSTIEEQ AKTFLDKENH
              310 320 330 340 350
EAEDLFYQSS LASWNYNTNI TEENVQNMNN AGDKWSAFLK EQSTLAQMYP
              360 370 380 390 400
LQEIQNLTVK LQLQALQQNG SSVLSEDKSK RLNTILNTMS TIYSTGKVCN
              410 420 430 440 450
PDNPQECLLL EPGLNEIMAN SLDYNERLWA WESWRSEVGK QLRPLYEEYV
              460 470 480 490 500
VLKNEMARAN HYEDYGDYWR GDYEVNGVDG YDYSRGQLIE DVEHTFEEIK
              510 520 530 540 550
PLYEHLHAYV RAKLMNAYPS YISPIGCLPA HLLGDMWGRF WTNLYSLTVP
              560 570 580 590 600
FGQKPNIDVT DAMVDQAWDA QRIFKEAEKF FVSVGLPNMT QGFWENSMLT
              610 620 630 640 650
DPGNVQKAVC HPTAWDLGKG DERILMCTKV TMDDELTAHH EMGHIQYDMA
              660 670 680 690 700
YAAQPFLLRN GANEGFHEAV GEIMSLSAAT PKHLKSIGLL SPDFQEDNET
              710 720 730 740 750
EINFLLKQAL TIVGTLPFTY MLEKWRWMVF KGEIPKDQWM KKWWEMKREI
              760 770 780 790 800
VGVVEPVPHD ETYCDPASLF HVSNDYSFIR YYTRTLYQFQ FQEALCQAAK
              810 820 830 840 850
HEGPLHKCDI SNSTEAGQKL FNMLRLGKSE PWTLALENVV GAKNMNVRPL
              860 870 880
LNYFEPLFTW LKDQNKNSFV GWSTDWSPYA GGHHHHHH
SEQ ID NO: 5
                10 20 30 40 50
MGWSCIILFL VATATGVHSR VQPTESIVRF PNITNLCPFG EVENATRFAS
                60 70 80 90 100
VYAWNRKRIS NCVADYSVLY NSASFSTFKC YGVSPTKIND LCFTNVYADS
              110 120 130 140 150
FVIRGDEVRQ IAPGQTGKIA DYNYKLPDDF TGCVIAWNSN NLDSKVGGNY
              160 170 180 190 200
NYLYRLERKS NLKPFERDIS TEIYQAGSTP CNGVEGENCY FPLQSYCFQP
              210 220 230 240 250
TNGVGYQPYR VVVLSFELLH APATVCGPKK STNLVKNKCV NEGGSGLNDI
              260 270 280 290 300
FEAQKIEWHE GGSENLYFQG GSGGGGSGGG GSSQSTIEEQ AKTFLDKENH
              310 320 330 340 350
EAECLFYQSS LASWNYNTNI TEENVQNMNN AGDKWSAFLK EQSTLAQMYP
              360 370 380 390 400
LQEIQNLTVK LQLQALQQNG SSVLSEDKSK RLNTILNTMS TIYSTGKVCN
              410 420 430 440 450
PDNPQECLLL EPGLNEIMAN SLDYNERLWA WESWRSEVGK QLRPLYEEYV
              460 470 480 490 500
VLKNEMARAN HYEDYGDYWR GDYEVNGVDG YDYSRGQLIE DVEHTFEEIK
              510 520 530 540 550
PLYEHLHAYV RAKLMNAYPS YISPIGCLPA HLLGDMWGRF WTNLYSLTVP
              560 570 580 590 600
FGQKPNIDVT DAMVDQAWDA QRIFKEAEKF FVSVGLPNMT QGFWENSMLT
              610 620 630 640 650
DPGNVQKAVC HPTAWDLGKG DFRILMCTKV TMDDELTAHH EMGHIQYDMA
              660 670 680 690 700
YAAQPELLRN GANEGFHEAV GEIMSLSAAT PKHLKSIGLL SPDFQEDNET
              710 720 730 740 750
EINFLLKQAL TIVGTLPFTY MLEKWRWMVF KGEIPKDQWM KKWWEMKREI
              760 770 780 790 800
VGVVEPVPHD ETYCDPASLF HVSNDYSFIR YYTRTLYQFQ FQEALCQAAK
              810 820 830 840 850
HEGPLHKCDI SNSTEAGQKL FNMLRLGKSE PWTLALENVV GAKNMNVRPL
              860 870 880
LNYFEPLFTW LKDQNKNSFV GWSTDWSPYA GGHHHHHH
```

### Enumerated Embodiments

The following enumerated embodiments are provided, the numbering of which is not be construed as designating levels of importance.

Embodiment 1 provides a method for detecting neutralizing antibodies of a viral protein in a biological sample comprising a first set of antibodies and a second set of antibodies, the method comprising: (a) contacting the biological sample with a modified form of the viral protein, thereby binding the first set of antibodies from the biological sample to the modified form of the viral protein, thereby generating a first antibody-protein complex; (b) contacting the biological sample from step (a) with an unmodified viral protein, thereby binding the second set of antibodies from the biological sample to the unmodified viral protein, thereby generating a second antibody-protein complex; (c) separating the first antibody-protein complex from the second antibody-protein complex; and (d) measuring the second antibody-protein complex, thereby detecting the neutralizing antibodies.

Embodiment 2 provides the method of embodiment 1, wherein the unmodified viral protein is immobilized on a surface.

Embodiment 3 provides the method of embodiment 2, wherein the second antibody-protein complex remains immobilized on the surface after the separating of step (c).

Embodiment 4 provides the method of embodiment 3, wherein the separating of step (c) occurs by removing the mixture of the biological sample and the modified form of the viral protein from contacting with the surface.

Embodiment 5 provides the method of any one of embodiments 1 to 4, wherein the unmodified viral protein of step (b) is provided on a microarray.

Embodiment 6 provides the method of any one of embodiments 1 to 5, wherein the modified form of the viral protein of step (a) is provided in a solution comprising the biological sample.

Embodiment 7 provides the method of embodiment 6, wherein the contacting of step (a) occurs in the solution.

Embodiment 8 provides the method of embodiment 6 or 7, wherein the contacting of step (a) comprises mixing the biological sample with the modified form of the viral protein provided in the solution.

Embodiment 9 provides the method of any one of embodiments 2 to 8, wherein the method further comprises washing the surface prior to the measuring of step (d).

Embodiment 10 provides the method of any one of embodiments 1 to 9, wherein step (d) comprises measuring the amount of the second antibody-protein complex.

Embodiment 11 provides the method of any one of embodiments 1 to 10, wherein step (d) comprises contacting the second antibody-protein complex with a set of labeled anti-IgG antibodies.

Embodiment 12 provides the method of embodiment 11, wherein the label on the set of anti-IgG comprises a fluorescent label.

Embodiment 13 provides the method of any one of embodiments 1 to 12, wherein the modified form of the viral protein possesses diminished affinity for neutralizing antibodies of the viral protein.

Embodiment 14 provides the method of any one of embodiments 1 to 13, wherein the modified form of the viral protein comprises at least one of a blocked neutralizing antibody epitope and an altered neutralizing antibody epitope.

Embodiment 15 provides the method of embodiment 14, wherein the modified form of the viral protein comprises an altered neutralizing antibody epitope comprising one or more amino acid variations selected from a mutation, an insertion, a deletion, a chemical modification, a truncation, cleavage, or any combination thereof.

Embodiment 16 provides the method of embodiment 15, wherein the modified form of the viral protein comprises a blocked neutralizing antibody epitope and a blocking group.

Embodiment 17 provides the method of embodiment 16, wherein the blocking group is covalently coupled to the blocked neutralizing antibody epitope.

Embodiment 18 provides the method of embodiment 17, wherein the blocking group is covalently coupled to the blocked neutralizing antibody epitope by a disulfide bond.

Embodiment 19 provides the method of any one of embodiments 16 to 18, wherein the blocking group is covalently coupled to the modified viral protein by a first linker.

Embodiment 20 provides the method of embodiment 19, wherein the first linker is a polypeptide linker.

Embodiment 21 provides the method of any one of embodiments 16 to 20, wherein the blocking group comprises a receptor protein having an affinity for the viral protein.

Embodiment 22 provides the method of any one of embodiments 1 to 21, wherein the viral protein comprises at least a portion of a coronavirus viral surface protein.

Embodiment 23 provides the method of any one of embodiments 1 to 22, wherein the viral protein comprises at least a portion of a coronavirus viral spike protein.

Embodiment 24 provides the method of any one of embodiments 1 to 23, wherein the viral protein comprises at least a portion of a SARS-CoV-2 surface protein.

Embodiment 25 provides the method of embodiments 1 to 24, wherein the viral protein comprises at least a portion of a receptor binding domain (RBD) of a SARS-CoV-2 spike protein.

Embodiment 26 provides the method of any one of embodiments 1 to 25, wherein the viral protein comprises an amino acid sequence having at least 90%, at least 95%, at least 96%, at least 97%, at least 98%, at least 99%, or 100% sequence identity to amino acids nos. 319 to 541 of SEQ ID NO: 1.

Embodiment 27 provides the method of any one of embodiments 1 to 26, wherein the modified viral protein comprises at least a portion of a SARS CoV-2 surface protein and at least a portion of an angiotensin converting enzyme 2 (ACE2) protein.

Embodiment 28 provides the method of embodiment 27, wherein the at least a portion of the SARS CoV-2 surface protein comprises an amino acid sequence having at least 90%, at least 95%, at least 96%, at least 97%, at least 98%, at least 99%, or 100% sequence identity to the amino acids nos. 20 to 242 of SEQ ID NO:4, and the at least a portion of the ACE2 protein comprises an amino acid sequence having at least 90%, at least 95%, at least 96%, at least 97%, at least 98%, at least 99%, or 100% sequence identity to amino acids nos. 284 to 880 of SEQ ID NO:4.

Embodiment 29 provides the method of any one of embodiments 1 to 28, wherein the modified viral protein comprises an amino acid sequence having at least 90%, at least 95%, at least 96%, at least 97%, at least 98%, at least 99%, or 100% sequence identity to amino acids nos 20 to 880 of SEQ ID NO:4.

Embodiment 30 provides the method of any one of embodiments 1 to 29, wherein the modified viral protein comprises a sequence having at least 90%, at least 95%, at least 96%, at least 97%, at least 98%, at least 99%, or 100% sequence identity to amino acids nos. 20 to 880 of SEQ ID NO:5.

Embodiment 31 provides the method of embodiment 24 or embodiment 25, wherein the SARS-CoV-2 surface protein viral comprises at least a portion of a SARS-CoV-2 variant spike protein.

Embodiment 32 provides the method of any one of embodiments 24, 25, and 31, wherein the SARS-CoV-2 surface protein comprises at least a portion of a receptor binding domain (RBD) of SARS-CoV-2 variant spike protein.

Embodiment 33 provides the method of any one of embodiments 24, 25, 31, and 32, wherein the SARS-CoV-2 surface protein comprises at least a portion of a SARS CoV-2 variant surface protein and at least a portion of an angiotensin converting enzyme 2 (ACE2) protein.

Embodiment 34 provides the method of any one of embodiments 24, 25, 31 to 33, wherein the modified viral protein comprises at least a portion of a SARS CoV-2 variant spike protein and at least a portion of an angiotensin converting enzyme 2 (ACE2) protein, and wherein the at least a portion of the ACE2 protein comprises an amino acid sequence having at least 90%, at least 95%, at least 96%, at least 97%, at least 98%, at least 99%, or 100% sequence identity to amino acids nos. 284 to 880 of SEQ ID NO:4.

Embodiment 35 provides the method of any one of embodiments 1 to 34, wherein the modified viral protein comprises part of a multivalent modified protein complex.

Embodiment 36 provides the method of embodiment 35, wherein the multivalent protein complex comprises two or more modified viral proteins.

Embodiment 37 provides the method of embodiment 35 or 36, wherein the two or more modified viral proteins are connected covalently or non-covalently via a scaffold or a multimerizing domain.

Embodiment 38 provides the method of embodiment 37, wherein each of the two or more modified proteins is independently attached to the scaffold or the multimerizing domain via a second linker.

Embodiment 39 provides the method of embodiment 38, wherein the second linker comprises a polypeptide, a water-soluble oligomer, an alkylene chain, a branched linker, or an organic polymer or any combination thereof.

Embodiment 40 provides the method of embodiment 38, wherein the second linker comprises a biotin molecule.

Embodiment 41 provides the method of any one of embodiments 37 to 40, wherein the two or more modified viral proteins are connected covalently or non-covalently via a scaffold comprising a protein, a polymer, or any combination thereof.

Embodiment 42 provides the method of any one of embodiments 37 to 41, wherein the scaffold comprises a biotin-binding protein, or a portion thereof.

Embodiment 43 provides the method of any one of embodiments 37 to 42, wherein the scaffold comprises avidin, neutravidin, or streptavidin, or a portion thereof.

Embodiment 44 provides the method of any one of embodiments 37 to 43, wherein the scaffold comprises neutravidin, or a portion thereof.

Embodiment 45 provides the method of any one of embodiments 37 to 44, wherein a biotin molecule connects each of the two or more modified proteins to the scaffold via a noncovalent interaction.

Embodiment 46 provides the method of any one of embodiments 37 to 45, wherein the scaffold comprises a biomolecule.

Embodiment 47 provides the method of any one of embodiments 37 to 46, wherein the scaffold comprises a water-soluble polymer.

Embodiment 48 provides the method of any one of embodiments 37 to 39, wherein the two or more modified viral proteins are connected covalently or non-covalently via a multimerizing domain.

Embodiment 49 provides the method of any one of embodiments 37 to 39, and 48, wherein the multimerizing domain comprises a T4 trimerization domain.

Embodiment 50 provides the method of any one of embodiments 36 to 49, wherein each of the two or more modified proteins independently comprises at least a portion of a coronavirus viral surface protein and at least a portion of an angiotensin converting enzyme 2 (ACE2) protein.

Embodiment 51 provides the method of embodiment 50, wherein the at least a portion of a coronavirus viral surface protein comprises at least a portion of a coronavirus spike protein.

Embodiment 52 provides the method of embodiment 50, wherein the at least a portion of a coronavirus viral surface protein comprises at least a portion of a receptor binding domain (RBD) of a coronavirus spike protein.

Embodiment 53 provides the method of any one of embodiments 50 to 52, wherein the coronavirus comprises SARS-COV-2.

Embodiment 54 provides the method of any one of embodiments 50 to 53, wherein the at least a portion of the ACE2 protein comprises an amino acid sequence having at least 90%, at least 95%, at least 96%, at least 97%, at least 98%, at least 99%, or 100% sequence identity to amino acids nos. 284 to 880 of SEQ ID NO:4.

Embodiment 55 provides the method of any one of embodiments 36 to 54, wherein each of the two or more modified proteins independently comprises at a sequence having at least 90%, at least 95%, at least 96%, at least 97%, at least 98%, at least 99%, or 100% sequence identity to amino acids nos. 20 to 880 of SEQ ID NO:4.

Embodiment 56 provides the method of any one of embodiments 36 to 54, wherein each of the two or more modified proteins independently comprises a sequence having at least 90%, at least 95%, at least 96%, at least 97%, at least 98%, at least 99%, or 100% sequence identity to amino acids nos. 20 to 880 of SEQ ID NO:5.

Embodiment 57 provides the method of any one of embodiments 1 to 56, wherein the modified viral protein binds to one or more antibodies.

Embodiment 58 provides the method of any one of embodiments 1 to 57, wherein the first set of antibodies comprises a non-neutralizing antibody.

Embodiment 59 provides the method of any one of embodiments 1 to 58, wherein the second set of antibodies comprises neutralizing antibodies with respect to the viral protein.

Embodiment 60 provides the method of any one of embodiments 1 to 59, wherein the biological sample comprises a blood sample taken from a subject.

Embodiment 61 provides the method of embodiment 60, wherein the subject was previously infected with a viral pathogen that is the origin of the viral protein or received a vaccine for the viral pathogen.

Embodiment 62 provides the method of embodiment 60, wherein the subject previously contracted SARS-COV-2 or a variant thereof.

Embodiment 63 provides the method of embodiment 60, wherein the subject previously received a vaccine for SARS-COV-2 or a variant thereof.

Embodiment 60 provides a multivalent modified protein complex comprising two or more unnatural proteins, wherein each of the two or more unnatural proteins independently comprises (a) a viral protein, (b) a receptor protein having an affinity for the viral protein, and (c) a first linker connecting the viral protein and the receptor protein, wherein each of the two or more unnatural proteins selectively binds to non-neutralizing antibodies of the viral protein over neutralizing antibodies of the viral protein.

Embodiment 65 provides the multivalent modified protein complex of embodiment 64, wherein the two or more unnatural proteins are connected covalently or non-covalently via a scaffold or a multimerizing domain.

Embodiment 66 provides the multivalent modified protein complex of embodiment 64 or 65, wherein each of the two or more unnatural proteins is independently attached to the scaffold or the multimerizing domain via a second linker.

Embodiment 67 provides the multivalent modified protein complex of embodiment 66, wherein the second linker comprises a polypeptide, a water-soluble oligomer, an alkylene chain, a branched linker, or an organic polymer or any combination thereof.

Embodiment 68 provides the multivalent modified protein complex of embodiment 66, wherein the second linker comprises a biotin molecule.

Embodiment 69 provides the multivalent modified protein complex of any one of embodiments 64 to 68, wherein the two or more unnatural proteins are connected covalently or non-covalently via a scaffold comprising a protein, a polymer, or any combination thereof.

Embodiment 70 provides the multivalent modified protein complex of any one of embodiments 64 to 69, wherein the scaffold comprises a biotin-binding protein.

Embodiment 71 provides the multivalent modified protein complex of any one of embodiments 64 to 70, wherein the scaffold comprises avidin, neutravidin, or streptavidin.

Embodiment 72 provides the multivalent modified protein complex of any one of embodiments 64 to 71, wherein the scaffold comprises neutravidin.

Embodiment 73 provides the multivalent modified protein complex of embodiment 72, wherein a biotin molecule connects each of the two or more unnatural proteins to the scaffold via a noncovalent interaction.

Embodiment 74 provides the multivalent modified protein complex of any one of embodiments 64 to 73, wherein the scaffold comprises a biomolecule.

Embodiment 75 provides the multivalent modified protein complex of any one of embodiments 64 to 74, wherein the scaffold comprises a water-soluble polymer.

Embodiment 76 provides the multivalent modified protein complex of any one of embodiments 64 to 67, wherein the two or more unnatural proteins are connected covalently or non-covalently via a multimerizing domain.

Embodiment 77 provides the multivalent modified protein complex of any one of embodiments 64 to 67 and 76, wherein the multimerizing domain comprises a T4 trimerization domain.

Embodiment 78 provides the multivalent modified protein complex of any one of embodiments 64 to 77, wherein each of the two or more unnatural proteins comprises at least 1-order of magnitude greater binding affinity for the non-neutralizing antibodies than for the neutralizing antibodies.

Embodiment 79 provides the multivalent modified protein complex of any one of embodiments 64 to 78, wherein the neutralizing antibodies comprise at least 1-order of magnitude lower binding affinity for the each of the two or more unnatural proteins than for the viral protein in an absence of the receptor protein and the linker.

Embodiment 80 provides the multivalent modified protein complex of any one of embodiments 64 to 79, wherein the viral protein comprises a receptor binding domain (RBD), and wherein the neutralizing antibodies possess affinities for the RBD of the viral protein.

Embodiment 81 provides the multivalent modified protein complex of any one of embodiments 64 to 80, wherein the affinity of the receptor protein for the viral protein is for at least a portion of the RBD of the viral protein.

Embodiment 82 provides the multivalent modified protein complex of any one of embodiments 64 to 81, wherein the viral protein comprises at least a portion of a wild type viral protein.

Embodiment 83 provides the multivalent modified protein complex of any one of embodiments 64 to 82, wherein the viral protein comprises at least a portion of a viral surface protein.

Embodiment 84 provides the multivalent modified protein complex of any one of embodiments 64 to 83, wherein the viral protein comprises at least a portion of a receptor binding domain (RBD) of the viral surface protein.

Embodiment 85 provides the multivalent modified protein complex of any one of embodiments 64 to 84, wherein the viral protein comprises at least a portion of a protein selected from the group consisting of: an influenza A hemagglutinin protein, an influenza A neuraminidase protein, a MERS-CoV spike protein, a SARS-CoV2 spike protein, a zikavirus E protein, a polyomavirus VP1 protein, and an HIV Env protein.

Embodiment 86 provides the multivalent modified protein complex of embodiment 83 or 84, wherein the viral surface protein is a coronavirus viral surface protein.

Embodiment 87 provides the multivalent modified protein complex of embodiment 86, wherein the coronavirus viral surface protein is a surface protein of SARS-CoV2 or a variant thereof.

Embodiment 88 provides the multivalent modified protein complex of any one of embodiments 64 to 87, wherein the viral protein comprises an amino acid sequence having at least 90%, at least 95%, at least 96%, at least 97%, at least 98%, at least 99%, or 100% sequence identity to amino acids nos. 319 to 541 of SEQ ID NO: 1.

Embodiment 89 provides the multivalent modified protein complex of any one of embodiments 83 to 88, wherein the receptor protein comprises at least a portion of a natural binding target for the viral surface protein.

Embodiment 90 provides the multivalent modified protein complex of any one of embodiments 64 to 89, wherein the receptor protein comprises at least a portion of an angiotensin converting enzyme 2 (ACE2).

Embodiment 91 provides the multivalent modified protein complex of embodiment 90, wherein the receptor protein comprises a soluble fragment of the angiotensin converting enzyme 2 (ACE2).

Embodiment 92 provides the multivalent modified protein complex of any one of embodiments 64 to 91, wherein the receptor protein comprises an amino acid sequence having at least 90%, at least 95%, at least 96%, at least 97%, at least 98%, at least 99%, or 100% sequence identity to amino acids nos. 18 to 614 of SEQ ID NO:2.

Embodiment 93 provides the multivalent modified protein complex of any one of embodiments 64 to 92, wherein the first linker comprises a polypeptide linker.

Embodiment 94 provides the multivalent modified protein complex of any one of embodiments 64 to 93, wherein the first linker connects the N-terminus of the receptor protein to the C-terminus of the viral protein.

Embodiment 95 provides the multivalent modified protein complex of any one of embodiments 64 to 94, wherein the first linker comprises a -GGGGS- pentapeptide motif (SEQ ID NO: 6).

Embodiment 96 provides the multivalent modified protein complex of any one of embodiments 64 to 95, wherein the first linker comprises an endopeptidase recognition sequence.

Embodiment 97 provides the multivalent modified protein complex of any one of embodiments 64 to 96, wherein the viral protein, the first linker, and the receptor protein are encoded by a single expression construct.

Embodiment 98 provides the multivalent modified protein complex of any one of embodiments 64 to 97, wherein the viral protein, the linker, and the receptor protein are encoded by a continuous nucleic acid sequence.

Embodiment 99 provides the multivalent modified protein complex of any one of embodiments 64 to 98, wherein the viral protein of each of the two or more unnatural proteins adopts a substantially similar conformation as the viral protein without the receptor protein and the linker.

Embodiment 100 provides the multivalent modified protein complex of any one of embodiments 64 to 99, wherein each of the two or more unnatural protein independently comprises an amino acid sequence having at least 90%, at least 95%, at least 96%, at least 97%, at least 98%, at least 99%, or 100% sequence identity to the amino acids nos. 20 to 880 of SEQ ID NO: 4.

Embodiment 101 provides the multivalent modified protein complex of any one of embodiments 64 to 99, wherein the viral protein and the receptor protein are covalently linked by a disulfide bond.

Embodiment 102 provides the multivalent modified protein complex of embodiment 101, wherein the disulfide bond is formed between a first cysteine residue on the viral protein and a second cysteine residue on the receptor protein, and wherein at least one of the first cysteine residue and the second cysteine residue is not present in a wild-type sequence of the viral protein or the receptor protein.

Embodiment 103 provides the multivalent modified protein complex of embodiment 101 or 102, wherein the first cysteine residue is not present in the wild-type sequence of the viral protein and the second cysteine residue is not present in the wild-type sequence of the receptor protein.

Embodiment 104 provides the multivalent modified protein complex of any one of embodiments 101 to 103, wherein the disulfide bond is formed between a first thiol on the viral protein and a second thiol on the receptor protein.

Embodiment 105 provides the multivalent modified protein complex of embodiment 104, wherein at least one of the first thiol and the second thiol is part of an amino acid residue not present in the wild-type sequence of the viral protein or the receptor protein.

Embodiment 106 provides the multivalent modified protein complex of embodiment 104 or 105, wherein the first thiol is part of a first amino acid residue not present in the wild-type sequence of the viral protein and the second thiol is part of a second amino acid residue not present in the wild-type sequence of the receptor protein.

Embodiment 107 provides the multivalent modified protein complex of embodiment 106, wherein the first amino acid residue and the second amino acid residue are each cysteine.

Embodiment 108 provides the multivalent modified protein complex of any one of embodiments 104 to 107, wherein the first thiol is part of a first cysteine that replaces an amino acid residue corresponding to an amino acid residue between positions 142 and 242 of SEQ ID NO: 4.

Embodiment 109 provides the multivalent modified protein complex of any one of embodiments 104 to 108, wherein the second thiol is part of a second cysteine that replaces an amino acid residue corresponding to an amino acid residue between positions 284 and 340 of SEQ ID NO: 4.

Embodiment 110 provides the multivalent modified protein complex of any one of embodiments 104 to 109, wherein the first cysteine replaces an amino acid residue corresponding to position 196 of SEQ ID NO:4 and the second cysteine residue replaces an amino acid residue corresponding to position 304 of SEQ ID NO:4.

Embodiment 111 provides the multivalent modified protein complex of any one of embodiments 101 to 109, wherein the disulfide bond is an interchain disulfide bond.

Embodiment 112 provides the multivalent modified protein complex of any one of embodiments 64 to 99 or 101 to 111, wherein each of the two or more unnatural proteins comprises a sequence having at least 90%, at least 95%, at least 96%, at least 97%, at least 98%, at least 99%, or 100% sequence identity to amino acids nos. 20 to 880 of SEQ ID NO:5.

Embodiment 113 provides the multivalent modified protein complex of any one of embodiments 64 to 112, wherein at least one of the viral protein or the receptor protein is a full-length protein that has the same number of amino acids as the wild type sequence of the viral protein or the receptor protein.

Embodiment 114 provides the multivalent modified protein complex of any one of embodiments 64 to 112, wherein at least one of the viral protein or the receptor protein is a protein fragment of the wild type sequence of the viral protein or the receptor protein.

Embodiment 115 provides the multivalent modified protein complex of any one of embodiments 64 to 114, wherein each of the two or more unnatural proteins is substantially inert to human blood proteases.

Embodiment 116 provides the multivalent modified protein complex of any one of embodiments 64 to 115, wherein the affinity of the receptor protein for the viral protein equals to a dissociation constant (KD) of less than 1 μM, 800 nM, 600 nM, 400 nM, 200 nM, 150 nM, 100 nM, 80 nM, 60 nM, 50 nM, 40 nM, or 20 nM.

Embodiment 117 provides a method for detecting neutralizing antibody levels in a subject, the method comprising: (a) obtaining a biological sample taken from the subject; (b) detecting a concentration of neutralizing antibodies in the sample using a cell-free method; wherein step (b) is completed within one hour.

Embodiment 118 provides a method for detecting neutralizing antibody levels in a subject, the method comprising: (a) obtaining a biological sample taken from the subject; (b) adding to the biological sample an agent that selectively binds to non-neutralizing antibodies; and (c) measuring a concentration of neutralizing antibodies in the sample using a cell-free bioassay; wherein step (c) is completed within one hour.

Embodiment 119 provides the method of embodiment 117 or 118, wherein the method is completed within 30 minutes.

Embodiment 120 provides the method of embodiment 117 or 118, wherein the method is completed within 20 minutes.

Embodiment 121 provides the method of embodiment 117 or 118, wherein the method is completed within 15 minutes.

Embodiment 122 provides the method of any one of embodiments 117 to 121, wherein the neutralizing antibodies are specific for a viral protein.

Embodiment 123 provides the method of embodiment 122, wherein the subject was previously infected by a viral pathogen that is the origin of the viral protein or received a vaccine against the viral pathogen.

Embodiment 124 provides the method of embodiment 123, wherein the viral pathogen is a coronavirus.

Embodiment 125 provides the method of embodiment 123, wherein the viral pathogen is a SARS-COV-2 or a variant thereof.

Embodiment 126 provides the method of any one of embodiments 117 to 125, wherein the agent comprises a modified form of a viral protein.

Embodiment 127 provides the method of any one of embodiments 117 to 126, wherein the agent comprises a multivalent modified protein complex.

Embodiment 128 provides a method for detecting neutralizing antibody levels for two or more viral pathogens in a subject, the method comprising: obtaining a biological sample taken from the subject; adding to the biological sample a first agent that selectively binds to non-neutralizing antibodies against a first viral pathogen; measuring a concentration of neutralizing antibodies against the first viral pathogen in the biological sample using a cell-free bioassay; adding to the biological sample a second agent that selectively binds to non-neutralizing antibodies against a second viral pathogen; and measuring a concentration of neutralizing antibodies against the second viral pathogen in the biological sample using a cell-free bioassay.

Embodiment 129 provides the method of embodiment 128, wherein the two or more viral pathogens are variants of a viral pathogen.

Embodiment 130 provides a method for distinguishing neutralizing antibody levels for each of two or more variants of a viral pathogen in a subject, the method comprising: obtaining a biological sample taken from the subject; adding to the biological sample a first agent that selectively binds to non-neutralizing antibodies against a first variant of the viral pathogen; measuring a concentration of neutralizing antibodies against the first variant of the viral pathogen in the biological sample using a cell-free bioassay; adding to the biological sample a second agent that selectively binds to non-neutralizing antibodies against second variant of the viral pathogen; measuring a concentration of neutralizing antibodies against the second variant of the viral pathogen in the biological sample using a cell-free bioassay; and comparing the concentrations of neutralizing antibodies against the first variant of the viral pathogen and the concentration of neutralizing antibodies against the second variant of the viral pathogen, thereby distinguishing the neutralizing antibody levels for each of the two or more variants of a viral pathogen in the subject.

Embodiment 131 provides the method of embodiment 129 or 130, wherein the viral pathogen is a coronavirus.

Embodiment 132 provides the method of embodiment 129 or 130, wherein the viral pathogen is a SARS-COV-2.

Embodiment 133 provides the method of embodiment 129 or 130, wherein the variants of the viral pathogen comprises Wuhan variant, delta variant, BAI variant, or omicron variant of SARS-CoV-2 or any combination thereof.

Embodiment 134 provides a method for predicting an immune response to a viral pathogen in a subject, the method comprising: (a) obtaining a biological sample taken from the subject; (b) quantifying an observed level of neutralizing antibodies in the sample using a cell-free bioassay procedure comprising: (i) adding to the sample a first agent that selectively binds non-neutralizing antibodies against the viral pathogen thereby generating a pretreated sample; (ii) contacting the pretreated sample with a second agent that binds neutralizing antibodies against the viral pathogen; and (iii) detecting the neutralizing antibodies bound to the second agent; and comparing the observed level of neutralizing antibodies to a predetermined threshold level of neutralizing antibodies.

Embodiment 135 provides a method for immunizing a subject against a viral pathogen, the method comprising: (a) obtaining a biological sample taken from the subject; (b) quantifying an observed level of neutralizing antibodies in the sample using a cell-free bioassay; (c) comparing the observed level of neutralizing antibodies to a predetermined threshold level of neutralizing antibodies; and (d) if the observed level of neutralizing antibodies in the sample is lower than the predetermined threshold level of neutralizing antibodies, then administering to the subject a vaccine directed against the viral pathogen.

Embodiment 136 provides a method for treating a subject infected with a viral pathogen, the method comprising: (a) obtaining a biological sample taken from the subject; (b) quantifying an observed level of neutralizing antibodies in the sample using a cell-free bioassay; (c) comparing the observed level of neutralizing antibodies to a predetermined threshold level of neutralizing antibodies; and (d) if the observed level of neutralizing antibodies in the sample is lower than the predetermined threshold level of neutralizing antibodies, then administering to the subject an antiviral therapy directed against the viral pathogen.

Embodiment 137 provides the method of any one of embodiments 134 to 136, wherein the predetermined threshold level of neutralizing antibodies is a reference or standard level established by WHO (World Health Organization), Centers for Disease Control and Prevention (CDC), or other public health agencies.

Embodiment 138 provides the method of any one of embodiments 128 to 137, wherein the biological sample comprises a blood sample.

Embodiment 139 provides the method of embodiment 138, wherein the blood sample comprises whole blood.

Embodiment 140 provides the method of embodiment 138, wherein the blood sample comprises fingerstick blood.

Embodiment 141 provides the method of any one of embodiments 128 to 137, wherein the subject previously received a vaccine against the viral pathogen.

Embodiment 142 provides an unnatural protein comprising: (a) a viral protein, (b) a receptor protein having an affinity for the viral protein, and (c) a linker connecting the viral protein and the receptor protein, wherein the unnatural protein selectively binds to non-neutralizing antibodies of the viral protein over neutralizing antibodies of the viral protein.

Embodiment 143 provides the unnatural protein of embodiment 142, wherein the unnatural protein comprises at least 1-order of magnitude greater binding affinity for the non-neutralizing antibodies than for the neutralizing antibodies.

Embodiment 144 provides the unnatural protein of either embodiment 142 or 143, wherein the neutralizing antibodies comprise at least 1-order of magnitude lower binding affinity for the unnatural protein than for the viral protein in an absence of the receptor protein and the linker.

Embodiment 145 provides the unnatural protein of any one of embodiments 142 to 144, wherein the viral protein comprises a receptor binding domain (RBD), and wherein the neutralizing antibodies possess affinities for the RBD of the viral protein.

Embodiment 146 provides the unnatural protein of any one of embodiments 142 to 145, wherein the affinity of the receptor protein for the viral protein is for at least a portion of the RBD of the viral protein.

Embodiment 147 provides the unnatural protein of any one of embodiments 142 to 146, wherein the viral protein comprises at least a portion of a wild type viral protein.

Embodiment 148 provides the unnatural protein of any one of embodiments 142 to 147, wherein the viral protein comprises at least a portion of a viral surface protein.

Embodiment 149 provides the unnatural protein of any one of embodiments 142 to 148, wherein the viral protein comprises at least a portion of a receptor binding domain of the viral surface protein.

Embodiment 150 provides the unnatural protein of any one of embodiments 142 to 149, wherein the viral protein comprises at least a portion of a protein selected from the group consisting of: an influenza A hemagglutinin protein, an influenza A neuraminidase protein, a MERS-CoV spike protein, a SARS-CoV2 spike protein, a zikavirus E protein, a polyomavirus VP1 protein, and an HIV Env protein.

Embodiment 151 provides the unnatural protein of embodiments 148 or 149, wherein the viral surface protein is a coronavirus viral surface protein.

Embodiment 152 provides the unnatural protein of embodiment 151, wherein the coronavirus viral surface protein is a surface protein of SARS-CoV2.

Embodiment 153 provides the unnatural protein of any one of embodiments 142 to 152, wherein the viral protein comprises an amino acid sequence having at least 90%, at least 95%, at least 96%, at least 97%, at least 98%, at least 99%, or 100% sequence identity to amino acids nos. 319 to 541 of SEQ ID NO: 1.

Embodiment 154 provides the unnatural protein of any one of embodiments 148 to 152, wherein the receptor protein comprises at least a portion of a natural binding target for the viral surface protein.

Embodiment 155 provides the unnatural protein of any one of embodiments 142 to 154, wherein the receptor protein comprises at least a portion of an angiotensin converting enzyme 2 (ACE2).

Embodiment 156 provides the unnatural protein of embodiment 155, wherein the receptor protein comprises a soluble fragment of the angiotensin converting enzyme 2 (ACE2).

Embodiment 157 provides the unnatural protein of any of embodiments 142 to 156, wherein the receptor protein comprises an amino acid sequence having at least 90%, at least 95%, at least 96%, at least 97%, at least 98%, at least 99%, or 100% sequence identity to amino acids nos. 18 to 614 of SEQ ID NO:2.

Embodiment 158 provides the unnatural protein of any one of embodiments 142 to 157, wherein the linker comprises a polypeptide linker.

Embodiment 159 provides the unnatural protein of any one of embodiments 142 to 158, wherein the linker connects the N-terminus of the receptor protein to the C-terminus of the viral protein.

Embodiment 160 provides the unnatural protein of any one of embodiments 142 to 159, wherein the linker comprises a -GGGGS- pentapeptide motif (SEQ ID NO: 6).

Embodiment 161 provides the unnatural protein of any one of embodiments 142 to 160, wherein the linker comprises an endopeptidase recognition sequence.

Embodiment 162 provides the unnatural protein of any one of embodiments 142 to 161, wherein the viral protein, the linker, and the receptor protein are encoded by a single expression construct.

Embodiment 163 provides the unnatural protein of embodiment 162, wherein the viral protein, the linker, and the receptor protein are encoded by a continuous nucleic acid sequence.

Embodiment 164 provides the unnatural protein of any one of embodiments 142 to 163, wherein the viral protein of the unnatural protein adopts a substantially similar conformation as the viral protein without the receptor protein and the linker.

Embodiment 165 provides the unnatural protein of any one of embodiments 142 to 164, wherein the unnatural protein comprises an amino acid sequence having at least 90%, at least 95%, at least 96%, at least 97%, at least 98%, at least 99%, or 100% sequence identity to the amino acids nos. 20 to 880 of SEQ ID NO:4.

Embodiment 166 provides the unnatural protein of any one of embodiments 142 to 165, wherein the viral protein and the receptor protein are covalently linked by a disulfide bond.

Embodiment 167 provides the unnatural protein of embodiment 166, wherein the disulfide bond is formed between a first cysteine residue on the viral protein and a second cysteine residue on the receptor protein, and wherein at least one of the first cysteine residue and the second cysteine residue is not present in a wild-type sequence of the viral protein or the receptor protein.

Embodiment 168 provides the unnatural protein of embodiments 166 or 167, wherein the first cysteine residue is not present in the wild-type sequence of the viral protein and the second cysteine residue is not present in the wild-type sequence of the receptor protein.

Embodiment 169 provides the unnatural protein of any one of embodiments 142 to 168, wherein the unnatural protein is immobilized on a surface.

Embodiment 170 provides the unnatural protein of embodiment 169, wherein the unnatural protein is coupled to the surface by a biotin binding agent.

Embodiment 171 provides the unnatural protein of either embodiment 169 or 170, wherein the unnatural protein is randomly oriented on the surface.

Embodiment 172 provides the unnatural protein of any one of embodiments 142 to 171, wherein the unnatural protein is substantially inert to human blood proteases.

Embodiment 173 provides the unnatural protein of any one of embodiments 142 to 172, wherein the affinity of the receptor protein for the viral protein is at least about 1 μM.

Embodiment 174 provides the unnatural protein of any one of embodiments 142 to 173, wherein the affinity of the receptor protein for the viral protein equals to a dissociation constant (KD) of less than 1 μM, 800 nM, 600 nM, 400 nM, 200 nM, 150 nM, 100 nM, 80 nM. 60 nM, 50 nM, 40 nM, or 20 nM.

Embodiment 175 provides an unnatural protein comprising a viral protein and a receptor protein having a binding affinity for the viral protein, wherein the viral protein is covalently linked to the receptor protein via a disulfide bond between a first thiol on the viral protein and a second thiol on the receptor protein.

Embodiment 176 provides the unnatural protein of embodiment 175, wherein at least one of the first thiol and the second thiol is part of an amino acid residue not present in a wild-type sequence of the viral protein or the receptor protein.

Embodiment 177 provides the unnatural protein of embodiment 175 or 176, wherein the first thiol is part of a first amino acid residue not present in a wild-type sequence of the viral protein and the second thiol is part of a second amino acid residue not present in a wild-type sequence of the receptor protein.

Embodiment 178 provides the unnatural protein of embodiment 177, wherein the first amino acid residue and the second amino acid residue are each cysteine.

Embodiment 179 provides the unnatural protein of any one of embodiment 175 to 178, wherein the viral protein comprises at least a portion of a receptor binding domain of a viral surface protein.

Embodiment 180 provides the unnatural protein of any one of embodiments 175 to 179, wherein the viral protein comprises a viral protein selected from the group consisting of: an influenza A hemagglutinin protein, an influenza A neuraminidase protein, a MERS-CoV spike protein, a SARS-CoV2 spike protein, a zikavirus E protein, a polyomavirus VP1 protein, and an HIV Env protein.

Embodiment 181 provides the unnatural protein of any one of embodiments 175 to 180, wherein the receptor protein comprises a natural host receptor protein for the viral protein.

Embodiment 182 provides the unnatural protein of any one of embodiments 175 to 181, wherein the viral protein comprises at least a portion of a coronavirus viral surface protein.

Embodiment 183 provides the unnatural protein of any one of embodiments 175 to 182, wherein the viral protein comprises at least a portion of a SARS-CoV2 viral surface protein.

Embodiment 184 provides the unnatural protein of embodiment 183, wherein the receptor protein comprises at least a portion of an angiotensin converting enzyme 2 (ACE2).

Embodiment 185 provides the unnatural protein of embodiment 184, wherein the first thiol is part of a first cysteine that replaces an amino acid residue corresponding to an amino acid residue between positions 142 and 242 of SEQ ID NO: 4.

Embodiment 186 provides the unnatural protein of embodiments 184 or 185, wherein the second thiol is part of a second cysteine that replaces an amino acid residue corresponding to an amino acid residue between positions 284 and 340 of SEQ ID NO: 4.

Embodiment 187 provides the unnatural protein of any one of embodiments 184 to 186, wherein the first cysteine replaces an amino acid residue corresponding to position 196 of SEQ ID NO:4 and the second cysteine residue replaces an amino acid residue corresponding to position 304 of SEQ ID NO:4.

Embodiment 188 provides the unnatural protein of any of embodiments 175 to 187, wherein the disulfide bond is an interchain disulfide bond.

Embodiment 189 provides the unnatural protein of any of embodiments 175 to 188, wherein the N-terminus of the receptor protein is connected to the C-terminus of the viral protein by a linker.

Embodiment 190 provides the unnatural protein of embodiment 189, wherein the linker is a polypeptide linker, and the disulfide bond is an intrachain disulfide bond.

Embodiment 191 provides the unnatural protein of any one of embodiments 175 to 190, wherein at least one of the viral protein or the receptor protein is a full-length protein that has the same number of amino acids as the wild type sequence of the viral protein or the receptor protein.

Embodiment 192 provides the unnatural protein of any one of embodiments 175 to 190, wherein at least one of the viral protein or the receptor protein is a protein fragment of the wild type sequence of the viral protein or the receptor protein.

Embodiment 193 provides the unnatural protein of any one of embodiments 175 to 192, wherein the unnatural protein comprises an amino acid sequence having at least 90%, at least 95%, at least 96%, at least 97%, at least 98%, at least 99%, or 100% sequence identity to amino acid nos. 20 to 242 of SEQ ID NO:5.

Embodiment 194 provides the unnatural protein of any one of embodiments 175 to 193, wherein the unnatural protein comprises an amino acid sequence having at least 90%, at least 95%, at least 96%, at least 97%, at least 98%, at least 99%, or 100% sequence identity to amino acid nos. 284 to 880 of SEQ ID NO:5.

Embodiment 195 provides the unnatural protein of any one of embodiments 142 to 194, wherein the unnatural protein is immobilized on a surface.

Embodiment 196 provides a nucleic acid molecule having a sequence encoding the two or more unnatural proteins according to any one of embodiments 64 to 112 or the unnatural protein of any one of embodiments 142 to 195.

Embodiment 197 provides a vector comprising the nucleic acid molecule of embodiment 196.

Embodiment 198 provides a host cell comprising the nucleic acid molecule of embodiment 196 or the vector of embodiment 197.

Embodiment 199 provides a method, comprising culturing the host cell of embodiment 198 under conditions for production of the two or more unnatural proteins according to any one of embodiments 64 to 112 or the unnatural protein of any one of embodiments 142 to 195.

Embodiment 200 provides a testing kit comprising the multivalent modified protein complex of any one of embodiments 64 to 112 or the unnatural protein of any one of embodiments 142 to 195 and instructions specified for using the testing kit to detect neutralizing antibodies in a biological sample taken from a subject.

Embodiment 201 provides a method for detecting neutralizing antibodies of a viral protein in a biological sample, the method comprising: (a) contacting the biological sample with the viral protein, thereby binding a first set of antibodies from the biological sample to the viral protein; (b) measuring the first set of antibodies bound to the viral protein; (c) contacting the biological sample to a modified form of the viral protein, thereby binding a second set of antibodies from the biological sample to the modified form of the viral protein; (d) measuring the second set of antibodies bound to the modified form of the viral protein; and

(e) determining a difference between the measurement of the first set of antibodies from step (b) and the measurement of the second set of antibodies from step (d), thereby detecting the neutralizing antibodies in the biological sample.

Embodiment 202 provides the method of embodiment 201, wherein the modified form of the viral protein possesses diminished affinity for neutralizing antibodies of the viral protein.

Embodiment 203 provides the method of embodiment 201 or 202, wherein the difference corresponds to a difference in an abundance of the first set of antibodies and an abundance of the second set of antibodies.

Embodiment 204 provides the method of any one of embodiments 201 to 203, wherein the difference corresponds to a difference in neutralizing antibody abundance between the first set of antibodies and the second set of antibodies.

Embodiment 205 provides the method of any one of embodiments 201 to 204, wherein the modified form of the viral protein comprises at least one of a blocked neutralizing antibody epitope and an altered neutralizing antibody epitope.

Embodiment 206 provides the method of embodiment 205, wherein the modified form of the viral protein comprises an altered neutralizing antibody epitope comprising a mutation, a chemical modification, cleavage, or any combination thereof.

Embodiment 207 provides the method of embodiment 206, wherein the modified form of the viral protein comprises a blocked neutralizing antibody epitope and a blocking group.

Embodiment 208 provides the method of embodiment 207, wherein the blocking group is covalently coupled to the blocked neutralizing antibody epitope.

Embodiment 209 provides the method of embodiment 208, wherein the blocking group is covalently coupled to the blocked neutralizing antibody epitope by a disulfide bond.

Embodiment 210 provides the method of any one of embodiments 207 to 209, wherein the blocking group is covalently coupled to the modified viral protein by a polypeptide linker.

Embodiment 211 provides the method of embodiment 210, wherein the modified viral protein is expressed as a single expression construct comprising the blocked neutralizing antibody epitope, the polypeptide linker, and the blocking group.

Embodiment 212 provides the method of any one of embodiments 206 to 211, wherein the blocking group comprises a receptor protein of the viral protein.

Embodiment 213 provides the method of any one of embodiments 142 to 212, wherein the viral protein comprises a fragment of a full-length viral protein.

Embodiment 214 provides the method of any one of embodiments 142 to 213, wherein the viral protein comprises at least a portion of a coronavirus viral surface protein.

Embodiment 215 provides the method of any one of embodiments 142 to 214, wherein the viral protein comprises at least a portion of a SARS-CoV-2 surface protein.

Embodiment 216 provides the method of embodiment 215, wherein the viral protein comprises an amino acid sequence having at least 90%, at least 95%, at least 96%, at least 97%, at least 98%, at least 99%, or 100% sequence identity to amino acids nos. 319 to 541 of SEQ ID NO: 1.

Embodiment 217 provides the method of any one of embodiments 142 to 216, wherein the modified viral protein comprises at least a portion of a SARS CoV-2 surface protein and at least a portion of an angiotensin converting enzyme 2 (ACE2) protein.

Embodiment 218 provides the method of embodiment 217, wherein the at least a portion of the SARS CoV-2 surface protein comprises an amino acid sequence having at least 90%, at least 95%, at least 96%, at least 97%, at least 98%, at least 99%, or 100% sequence identity to amino acids 20 to 242 of SEQ ID NO:4, and the at least a portion of the ACE2 protein comprises an amino acid sequence having at least 90%, at least 95%, at least 96%, at least 97%, at least 98%, at least 99%, or 100% sequence identity to amino acids 284 to 880 of SEQ ID NO:4.

Embodiment 219 provides the method of any one of embodiments 142 to 218, wherein the viral protein of (a) and the modified form of the viral protein of (c) are provided on a microarray.

Embodiment 220 provides the method of any one of embodiments 142 to 219, wherein step (b) comprises contacting the first set of antibodies and a first set of labeled anti-IgG antibodies and step (d) comprises contacting the second set of antibodies with a second set of labeled anti-IgG antibodies.

Embodiment 221 provides the method of embodiment 220, wherein the label on either or both of the first set of anti-IgG antibodies and the second set of anti-IgG antibodies comprises a fluorescent label.

Embodiment 222 provides the method of any one of embodiments 201 to 221, wherein step (b) and step (d) occur simultaneously.

Embodiment 223 provides the method of any one of embodiments 201 to 222, wherein the modified viral protein comprises a sequence having at least 90% sequence identity to amino acids nos 20 to 880 of SEQ ID NO:4.

Embodiment 224 provides the method of any one of embodiments 201 to 222, wherein the modified viral protein comprises a sequence having at least 90% sequence identity to amino acids nos 20 to 880 of SEQ ID NO:5.

Embodiment 225 provides the method of any one of embodiments 201 to 224, wherein the first set of antibodies comprises neutralizing antibodies with respect to the viral protein.

Embodiment 226 provides the method of any one of embodiments 201 to 225, wherein the second set of antibodies comprises non-neutralizing antibodies with respect to the viral protein.

Embodiment 227 provides a method for detecting neutralizing antibodies of a viral protein in a biological sample comprising a first set of antibodies and a second set of antibodies, the method comprising: (a) contacting the biological sample with a modified form of the viral protein, thereby bringing the first set of antibodies from the biological sample in contact with the modified form of the viral protein, thereby generating a first antibody-protein complex; (b) contacting a mixture of the biological sample and the modified form of the viral protein from step (a) with an unmodified viral protein, thereby bringing the second set of antibodies from the biological sample in contact with the unmodified viral protein, thereby generating a second antibody-protein complex; (c) separating the first antibody-protein complex from the second antibody-protein complex; and (d) detecting the second antibody-protein complex, thereby detecting the neutralizing antibodies.

Embodiment 228 provides the method of embodiment 227, wherein the unmodified viral protein is immobilized on a surface.

Embodiment 229 provides the method of embodiment 228, wherein the second antibody-protein complex remains immobilized on the surface after the separating of step (c).

Embodiment 230 provides the method of embodiment 229, wherein the separating of step (c) occurs by removing the mixture of the biological sample and the modified form of the viral protein from contacting with the surface.

Embodiment 231 provides the method of any one of embodiments 227 to 230, wherein the unmodified viral protein of step (b) is provided on a microarray.

Embodiment 232 provides the method of any one of embodiments 227 to 231, wherein the modified form of the viral protein of step (a) is provided in a solution.

Embodiment 233 provides the method of embodiment 232, wherein the contacting of step (a) occurs in the solution.

Embodiment 234 provides the method of embodiment 232 or 233, wherein the contacting of step (a) comprises mixing the biological sample with the modified form of the viral protein provided in the solution.

Embodiment 235 provides the method of any one of embodiments 228 to 234, wherein the method further comprises washing the surface prior to the detecting of step (d).

Embodiment 236 provides the method of any one of embodiments 227 to 235, wherein step (d) comprises measuring the amount of the second antibody-protein complex.

What is claimed is:

1. A method for detecting a neutralizing antibody of a viral protein in a biological sample comprising a neutralizing antibody and a non-neutralizing antibody, the method comprising:
(a) contacting the biological sample with a modified form of the viral protein, thereby binding the non-neutralizing antibody from the biological sample to the modified form of the viral protein, thereby generating a non-neutralizing antibody-protein complex;
(b) contacting the biological sample from (a) with an unmodified form of the viral protein, thereby binding the neutralizing antibody from the biological sample to the unmodified form of the viral protein, thereby generating a neutralizing antibody-protein complex;
(c) separating the non-neutralizing antibody-protein complex from the neutralizing antibody-protein complex; and
(d) detecting the neutralizing antibody-protein complex, thereby detecting the neutralizing antibody.

2. The method of claim 1, wherein the modified form of the viral protein possesses diminished affinity for the neutralizing antibody compared to the non-neutralizing antibody.

3. The method of claim 1, wherein the modified form of the viral protein comprises a blocked neutralizing antibody epitope that is blocked by a blocking group.

4. The method of claim 3, wherein the blocking group is covalently coupled to the modified form of the viral protein.

5. The method of claim 3, wherein the blocking group comprises a portion of a receptor protein having an affinity for the viral protein.

6. The method of claim 1, wherein the viral protein comprises at least a portion of a SARS CoV-2 surface protein, and wherein the modified form of the viral protein comprises at least a portion of a SARS CoV-2 surface protein and at least a portion of an angiotensin converting enzyme 2 (ACE2) protein.

7. The method of claim 1, wherein the modified form of the viral protein is part of a multivalent modified protein complex comprising two or more unnatural proteins, wherein each of the two or more unnatural proteins independently comprises:
(a) the viral protein,
(b) a receptor protein domain having an affinity for the viral protein, and
(c) a linker connecting the viral protein and the receptor protein domain, wherein the two or more unnatural proteins selectively bind to non-neutralizing antibodies of the viral protein over neutralizing antibodies of the viral protein.

8. The method of claim 1, wherein the unmodified form of the viral protein is immobilized on a surface.

9. A multivalent modified protein complex comprising two or more unnatural protein domains, wherein each of the two or more unnatural protein domains independently comprises:
(a) a viral protein,
(b) a receptor protein domain having an affinity for the viral protein, and
(c) a linker connecting the viral protein and the receptor protein domain,
wherein each of the two or more unnatural protein domains selectively binds to a non-neutralizing antibody of the viral protein over a neutralizing antibody of the viral protein.

10. The multivalent modified protein complex of claim 9, wherein the two or more unnatural protein domains are covalently or noncovalently attached to a scaffold via an additional linker.

11. The multivalent modified protein complex of claim 10, wherein the scaffold comprises a protein, a polymer, or a combination thereof.

12. The multivalent modified protein complex of claim 10, wherein the additional linker comprises a polypeptide, a water-soluble oligomer, an alkylene chain, a branched linker, an organic polymer, or a combination thereof.

13. The multivalent modified protein complex of claim 10, wherein the additional linker comprises a biotin molecule that is coupled to a biotin-binding protein in the scaffold.

14. The multivalent modified protein complex of claim 9, wherein the two or more unnatural protein domains are connected covalently or non-covalently via a multimerizing domain.

15. The multivalent modified protein complex of claim 14, wherein the multimerizing domain comprises a T4 trimerization domain.

16. The multivalent modified protein complex of claim 9, wherein the two or more unnatural protein domains are comprised in a single polypeptide.

17. The multivalent modified protein complex of claim 9, wherein each of the two or more unnatural protein domains comprises at least 1-order of magnitude greater binding affinity for the non-neutralizing antibody than for the neutralizing antibody.

18. The multivalent modified protein complex of claim 9, wherein the neutralizing antibody comprises at least 1-order of magnitude lower binding affinity for the each of the two or more unnatural protein domains than for the viral protein in absence of the receptor protein domain and the linker.

19. The multivalent modified protein complex of claim 9, wherein the viral protein comprises a receptor binding domain (RBD), and wherein the neutralizing antibody possesses affinity for the RBD of the viral protein.

20. The multivalent modified protein complex of claim 9, wherein the viral protein comprises at least a portion of a protein selected from the group consisting of: an influenza A hemagglutinin protein, an influenza A neuraminidase protein, a MERS-CoV spike protein, a SARS-CoV2 spike protein, a zikavirus E protein, a polyomavirus VP1 protein, and an HIV Env protein.

21. The multivalent modified protein complex of claim 9, wherein the viral protein comprises at least a portion of a SARS CoV-2 surface protein, and wherein the receptor protein domain comprises at least a portion of an angiotensin converting enzyme 2 (ACE2).
